(19) United States
(12) Patent Application Publication
(10) Pub. No.: US 2024/0150306 A1
(43) Pub. Date: May 9, 2024

(54) COMPOUNDS WITH CLEAVABLE DISULFIDE MOIETIES

(71) Applicant: REINVIGORON THERATECH, INC., Dallas, TX (US)

(72) Inventor: Kejin ZHOU, Dallas, TX (US)

(21) Appl. No.: 18/373,890

(22) Filed: Sep. 27, 2023

Related U.S. Application Data
(60) Provisional application No. 63/410,444, filed on Sep. 27, 2022.

(51) Int. Cl. C07D 295/15; C07C 323/25; C07D 295/088

(52) U.S. Cl. CPC C07D 295/15; C07C 323/25; C07D 295/088

(57) ABSTRACT

Provided herein are compounds having one or more cleavable disulfide moieties and one or more hydrophobic tail moieties (HTM) for delivery of one or more therapeutic or diagnostic agents. In some embodiments, the one or more therapeutic or diagnostic agents are biologics. In some embodiments, the one or more therapeutic or diagnostic agents are delivered to cancer cells.

### BACKGROUND OF THE DISCLOSURE

Delivery of a drug, such as a nucleic acid drug or other large molecule and small molecule drug, to its target site is important to achieve the drug's therapeutic effect. In particular, delivery of a drug susceptible to enzymatic degradation or cannot cross cell membranes to reach an intracellular target presents substantially challenges.

### SUMMARY OF THE DISCLOSURE

Provided herein are compounds having one or more cleavable disulfide moieties and one or more hydrophobic tail moieties (HTM) for delivery of one or more therapeutic or diagnostic agents. In some embodiments, the one or more therapeutic or diagnostic agents are biologics. In some embodiments, the one or more therapeutic or diagnostic agents are delivered to cancer cells.

A first aspect of the present disclosure relates to a compound of Formula I, a stereoisomer or pharmaceutically acceptable salt thereof:

RₐR_{b}N-L₁-S—S-L₂-NR_{c}R_{d} (Formula I)

wherein L₁ and L₂ are each independently C₁₋₄ alkylenyl;

wherein at least one of Rₐ, R_{b}, R_{c} and R_{d} is independently a hydrophobic tail moiety (HTM) or combined with nitrogen on the same side of Formula I to form a 3-member to 8-member heterocyclic ring substituted with an HTM, and the wherein the remainder of Rₐ, R_{b}, R_{c} and R_{d} is independently a hydrogen, a C₁₋₄ alkyl, or combine with nitrogen on the same side of Formula I to form a 3-member to 8-member heterocyclic ring optionally substituted with a C₁₋₄ alkyl,

wherein HTM is a C₅₋₄₀ linear or branched alkyl, C₅₋₄₀ linear or branched alkenyl, or C₅₋₄₀ linear or branched alkynyl; or wherein HTM has a structure of Formula II:

-L-NR′R″ (Formula II)
wherein L is C₁₋₄ alkylenyl and at least one of R′ and R″ is a C₅₋₄₅ linear or branched alkyl, C₅₋₄₅ linear or branched alkenyl, or C₅₋₄₅ linear or branched alkynyl.

In some embodiments, L₁ and L₂ are each independently C₁₋₃ alkylenyl. In some embodiments, L₁ is ethylene. In some embodiments, L₂ is ethylene.

In a refinement of the first aspect, Rₐ and R_{b} is independently a hydrogen, a C₁₋₄ alkyl, or combine with nitrogen on the same side of Formula I to form a 3-member to 8-member heterocyclic ring optionally substituted with a C₁₋₄ alkyl.

In some embodiments, Rₐ and R_{b} are hydrogen.

In some embodiments, the Rₐ is hydrogen and R_{b} is a C₁₋₄ alkyl. In some embodiments, R_{b} is a butyl.

In some embodiments, Rₐ and R_{b} are each independently a C₁₋₄ alkyl. In some embodiments, Rₐ and R_{b} are methyl.

In some embodiments, Rₐ and R_{b} combine with nitrogen on the same side of Formula I to form a 3-member to 8-member heterocyclic ring. In some embodiments, the heterocyclic ring is substituted with a C₁₋₄ alkyl.

In some embodiments, the heterocyclic ring is a 5-member to 7-member ring. In some embodiments, the heterocyclic ring is a 6-member ring. In some embodiments, the heterocyclic ring is a diazinane ring. In some embodiments, the diazinane ring is a 1,4-diazinane ring. In some embodiments, the 1,4-diazinane ring is substituted with a C₁₋₄ alkyl at the 4 position.

In some embodiments, R_{c} is an HTM and R_{d} is a hydrogen or a C₁₋₄ alkyl. In some embodiments, R_{c} and R_{d} are each independently an HTM.

In some embodiments, R_{c} and R_{d} combined with nitrogen on the same side of Formula I to form a 3-member to 8-member heterocyclic ring substituted with an HTM. In some embodiments, the heterocyclic ring is a 5-member to 7-member ring. In some embodiments, the heterocyclic ring is a 6-member ring. In some embodiments, the heterocyclic ring is a diazinane ring. In some embodiments, the diazinane ring is a 1,4-diazinane ring. In some embodiments, the 1,4-diazinane ring is substituted with the HTM at the 4 position.

In another refinement of the first aspect, at least one of Rₐ and R_{b} is an HTM or Rₐ and R_{b} combine with nitrogen on the same side of Formula one to form a 3-member to 8-member heterocyclic ring substituted with an HTM; and at least one of R_{c} and R_{d} is an HTM or R_{c} and R_{d} combine with nitrogen on the same side of Formula one to form a 3-member to 8-member heterocyclic ring substituted with an HTM.

In some embodiments, at least one of Rₐ and R_{b} is an HTM, and wherein at least one of R_{c} and R_{d} is an HTM.

In some embodiments, Rₐ is an HTM and R_{b} is hydrogen or a C₁₋₄ alkyl. In some embodiments, R_{b} is hydrogen. In some embodiments, R_{b} is a C₁₋₄ alkyl.

In some embodiments, R_{c} is an HTM and R_{d} is hydrogen or a C₁₋₄ alkyl. In some embodiments, R_{d} is hydrogen. In some embodiments, R_{d} is a C₁₋₄ alkyl.

In some embodiments, the C₁₋₄ alkyl is methyl. In some embodiments, the C₁₋₄ alkyl is butyl.

In some embodiments, R_{c} and R_{d} are each independently an HTM. In some embodiments, Rₐ, R_{b}, R_{c} and R_{d} are each independently an HTM.

In some embodiments, at least one of Rₐ and R_{b} is an HTM, and R_{c} and R_{d} combine with nitrogen on the same side of Formula one to form a 3-member to 8-member heterocyclic ring substituted with an HTM.

In some embodiments, Rₐ is an HTM and R_{b} is hydrogen or a C₁₋₄ alkyl. In some embodiments, R_{b} is hydrogen. In some embodiments, R_{b} is a C₁₋₄ alkyl. In some embodiments, the C₁₋₄ alkyl is methyl. In some embodiments, the C₁₋₄ alkyl is butyl.

In some embodiments, Rₐ and R_{b} are each independently an HTM.

In some embodiments, Rₐ and R_{b} combine with nitrogen on the same side of Formula one to form a 3-member to 8-member first heterocyclic ring substituted with an HTM; and R_{c} and R_{d} combine with nitrogen on the same side of Formula one to form a second 3-member to 8-member heterocyclic ring substituted with an HTM.

In some embodiments, the first and second heterocyclic rings are each independently a 5-member to 7-member ring. In some embodiments, the first and second heterocyclic rings are each independently a 6-member ring. In some embodiments, the first and second heterocyclic rings are each independently a diazinane ring. In some embodiments, the diazinane ring is a 1,4-diazinane ring. In some embodiments, the 1,4-diazinane ring is substituted with the HTM at the 4 position.

In some embodiments of the first aspect, HTM is a C₅₋₄₀ linear or branched alkyl, C₅₋₄₀ linear or branched alkenyl, or C₅₋₄₀ linear or branched alkynyl. In some embodiments, HTM is a C₅₋₄₀ linear or branched alkyl.

In some embodiments, HTM is a C₅₋₃₀ linear alkyl. In some embodiments, HTM is a C₅₋₂₀ linear alkyl. In some embodiments, HTM is a C₈₋₂₀ linear alkyl. In some embodiments, HTM is a C₁₀₋₂₀ linear alkyl. In some embodiments, HTM is a C₁₂₋₂₀ linear alkyl. In some embodiments, HTM is a C14-20 linear alkyl. In some embodiments, HTM is a C₁₆₋₂₀ linear alkyl. In some embodiments, HTM is a C₁₆₋₁₈ linear alkyl.

In some embodiments, HTM is a C₁₀₋₃₀ branched alkyl. In some embodiments, the branched alkyl has one C₅₋₁₅ alkyl branch on a C₅₋₁₅ alkyl main chain.

In some embodiments, HTM is a C₁₅₋₄₀ branched alkyl. In some embodiments, the branched alkyl has two C₅₋₁₅ alkyl branches on a C₅₋₁₅ alkyl main chain.

In some embodiments, the linear or branched alkyl, alkenyl, or alkynyl has one or more intervening groups selected from ester —(C═O)—O—, amide —(C═O)—NR—, or carboxylate —O—(C═O)—O—. In some embodiments, one or more intervening groups is selected from ester —(C═O)—O— or amide —(C═O)—NR—.

In some embodiments, the one or more intervening group is inserted in the alkyl such that the acyl group is connect to nitrogen in Formula I or Formula II through an ethylene —CH₂—CH₂—. In some embodiments, the one or more intervening group is one or more ester group. In some embodiments, HTM has the structure of

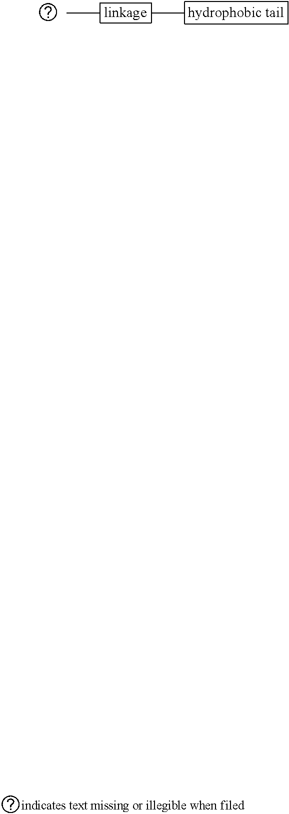

wherein the linkage is selected from

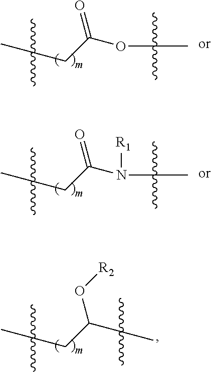

m is an integer of 1-6, R₁ is H, CH₃, or CH₂CH₃; and R₂ is H, CH₃, CH₂CH₃, or (C═O)CH₃; wherein the hydrophobic tail is selected from the group consisting of:

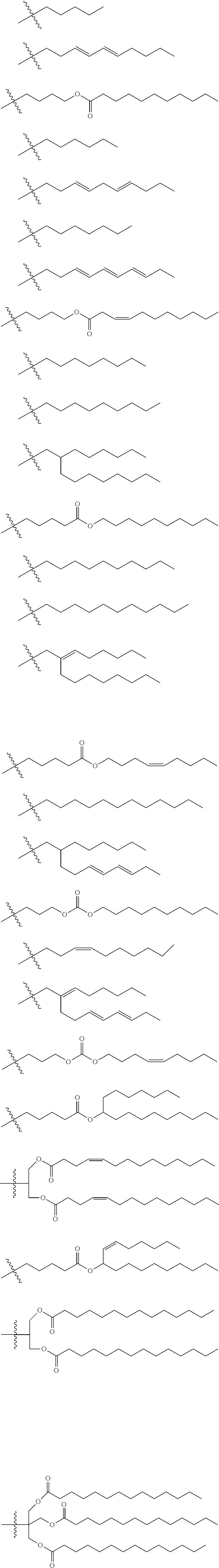

In some embodiments, the compounds according to the first aspect of the present disclosure are selected from the group consisting of:

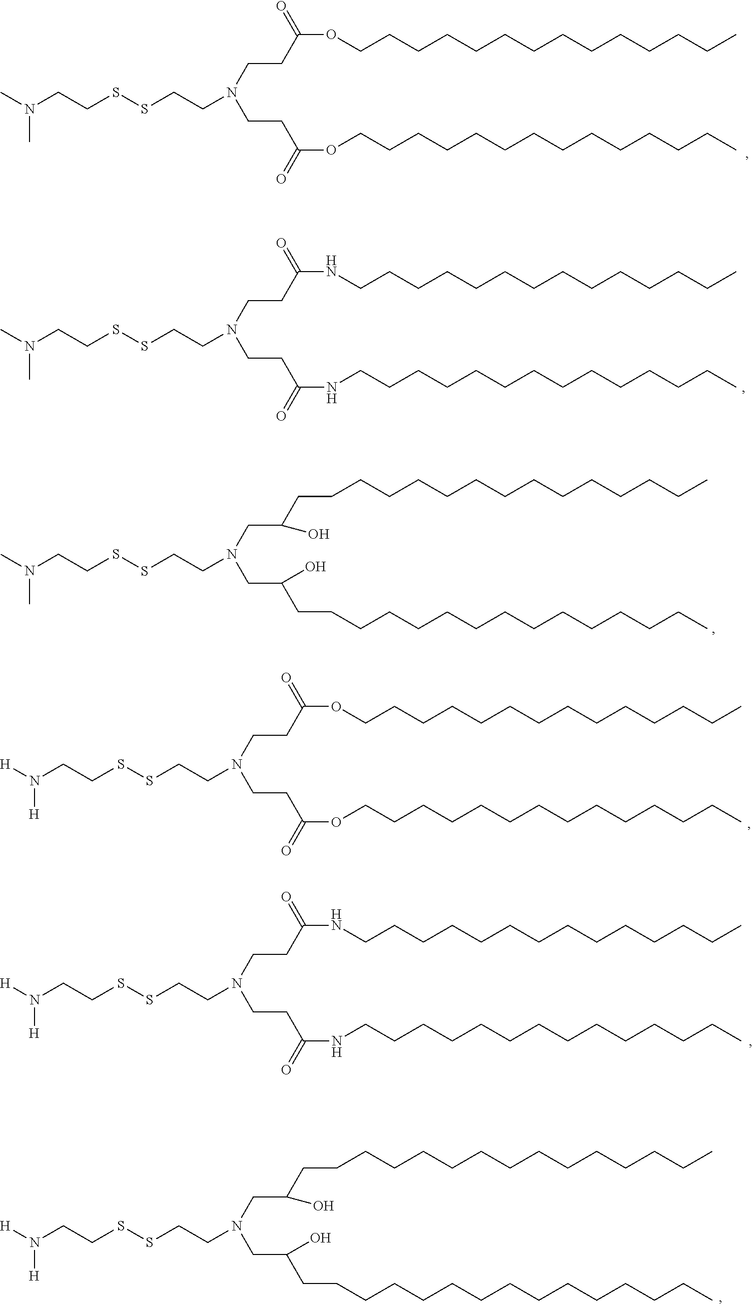

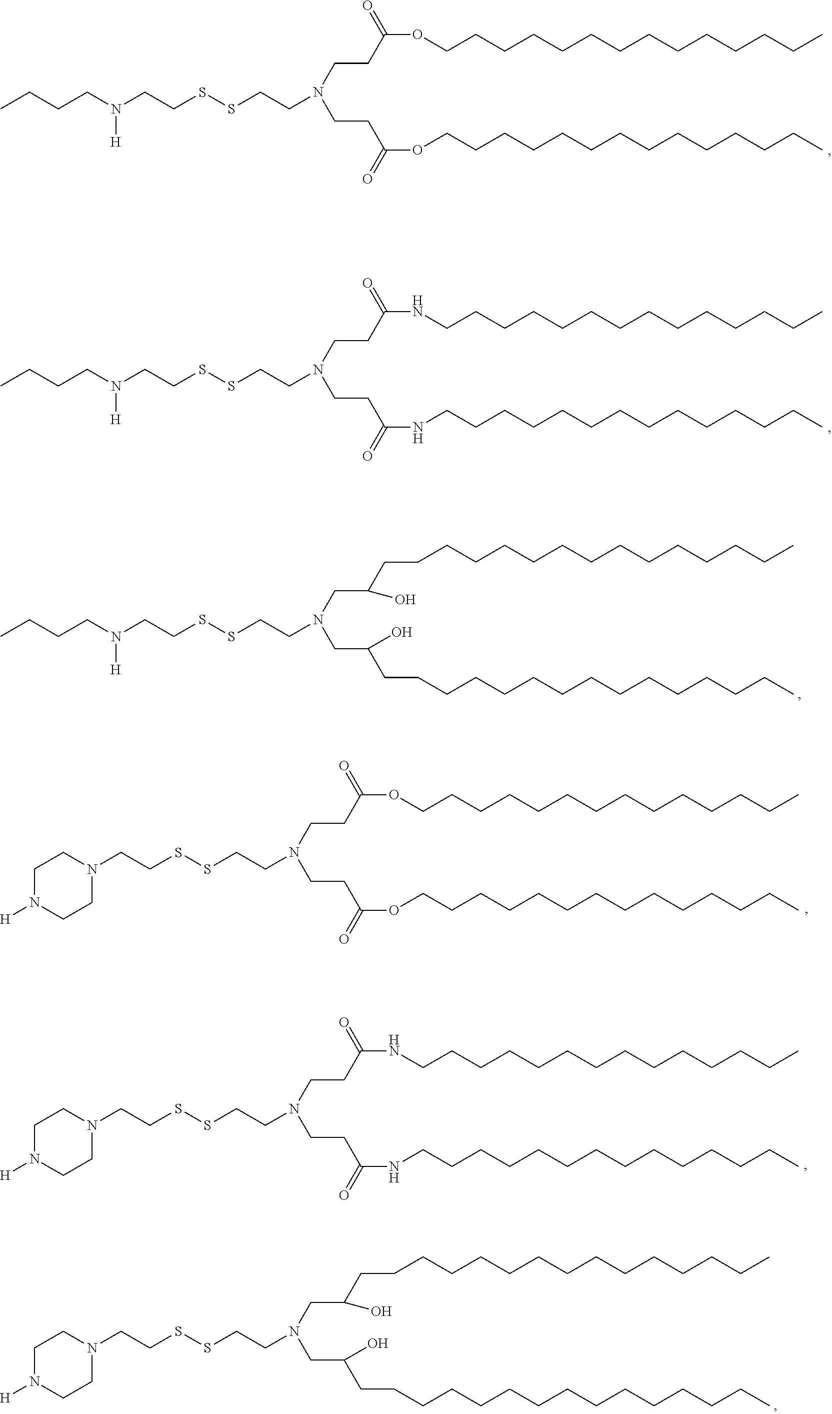

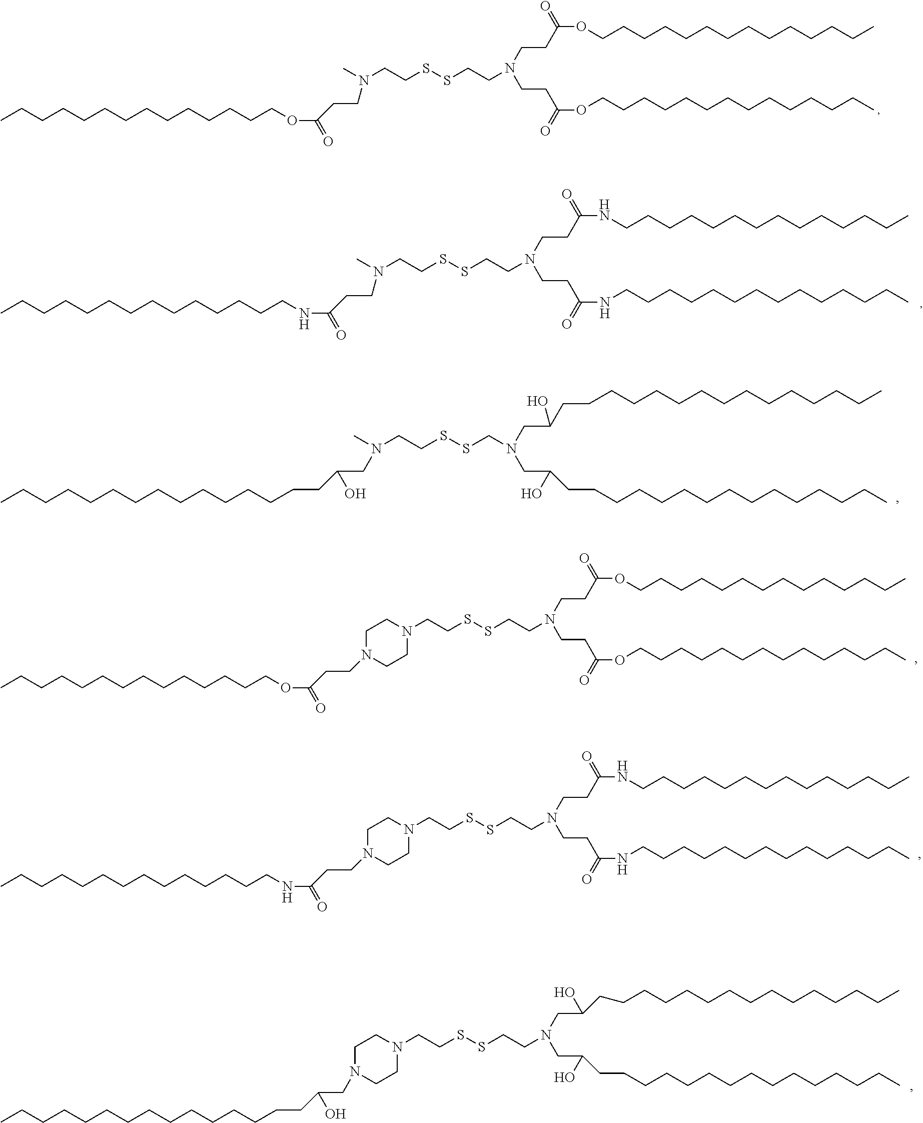

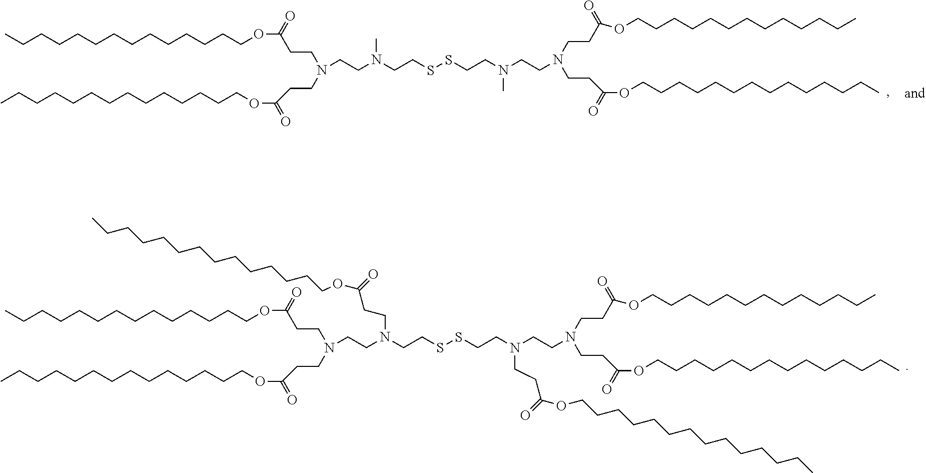

A second aspect of the present disclosure relates to a compound A compound of Formula III, a stereoisomer or pharmaceutically acceptable salt thereof:

(RₐR_{b}N-L₁-S—S-L₂-)nMVC (Formula III)

wherein MVC is a multi-valent core capable of connecting with two or more (RₐR_{b}N-L₁-S—S-L₂-), wherein n is an integer of 2-12;

wherein L₁ and L₂ are each independently C₁₋₄ alkylenyl;

wherein at least one of Rₐ and R_{b} is independently a hydrophobic tail moiety (HTM) or Rₐ and R_{b} combined with nitrogen to form a 3-member to 8-member heterocyclic ring substituted with an HTM, and the wherein the remainder of Rₐ, R_{b}, R_{c} and R_{d} is independently a hydrogen, a C₁₋₄ alkyl, or combine with nitrogen on the same side of Formula I to form a 3-member to 8-member heterocyclic ring optionally substituted with a C₁₋₄ alkyl,

wherein HTM is a C₅₋₄₀ linear or branched alkyl, C₅₋₄₀ linear or branched alkenyl, or C₅₋₄₀ linear or branched alkynyl; or wherein HTM has a structure of Formula II:

-L-NR′R″ (Formula II)
wherein L is C₁₋₄ alkylenyl and at least one of R′ and R″ is a C₅₋₄₅ linear or branched alkyl, C₅₋₄₅ linear or branched alkenyl, or C₅₋₄₅ linear or branched alkynyl.

In some embodiments, L₁ and L₂ are each independently C₁₋₃ alkylenyl. In some embodiments, L₁ is ethylene. In some embodiments, L₂ is ethylene.

In a first refinement of the second aspect of the present disclosure, MVC is a multivalent non-metal element. In some embodiments, oxygen or nitrogen. In some embodiments, the non-metal element is nitrogen.

In some embodiments, n is 2 and wherein the MVC is a divalent nitrogen —NR₁—, wherein R₁ is a hydrogen, a C₁₋₄ alkyl, or an HTM.

In some embodiments, the R₁ is a C1-4 alkyl. In some embodiments, the R₁ is a methyl. In some embodiments, the R₁ is HTM.

In some embodiments, n is 3 and wherein the MVC is a trivalent nitrogen.

In some embodiments, Rₐ is an HTM and R_{b} is a hydrogen or a C₁₋₄ alkyl. In some embodiments, Rₐ and R_{b} are each independently an HTM.

In some embodiments, Rₐ and R_{b} combined with nitrogen to form a 3-member to 8-member heterocyclic ring substituted with an HTM. In some embodiments, the heterocyclic ring is a 5-member to 7-member ring. In some embodiments, the heterocyclic ring is a 6-member ring. In some embodiments, the heterocyclic ring is a diazinane ring. In some embodiments, the diazinane ring is a 1,4-diazinane ring. In some embodiments, the 1,4-diazinane ring is substituted with the HTM at the 4 position.

In a second refinement of the second aspect of the present disclosure, the MVC is a multivalent radical of an organic compound having multiple functional groups, and optionally wherein the organic compound has a molecular weight of no more than 300, no more than 200 or no more than 150. In some embodiments, the functional groups are selected from —C(═O)—O—, —O—C(═O)—, —C(═O)—NR₂—, —NR₂—C(═O)—, or combinations thereof, wherein R₂ is hydrogen, a C₁₋₄ alkyl, C₁₋₄ alkenyl, or C₁₋₄ alkynyl. In some embodiments, the functional groups are —C(═O)—O—. In some embodiments, the functional groups are —O—C(═O)—. In some embodiments, the functional groups are —C(═O)-NR₂-. In some embodiments, the functional groups are -NR₂—C(═O)—.

In some embodiments, the organic compound is a nonaromatic compound. In some embodiments, the nonaromatic compound is an aliphatic compound. In some embodiments, the nonaromatic compound is a cyclic compound. In some embodiments, the cyclic nonaromatic compound is a heterocyclic compound. In some embodiments, the cyclic nonaromatic compound is a homocyclic compound. In some embodiments, the organic compound is a C₃₋₁₅ nonaromatic compound.

In some embodiments, the organic compound is an aromatic compound. In some embodiments, the aromatic compound is an homoaromatic compound. In some embodiments, the aromatic compound is a heteroaromatic compound. In some embodiments, the aromatic compound is a heterocyclic compound. In some embodiments, the aromatic compound is a monocyclic aromatic compound. In some embodiments, the aromatic compound is a polycyclic aromatic compound. In some embodiments, the organic compound is a C₅₋₁₅ aromatic compound.

In some embodiments, the MVC is a divalent radical of the organic compound. In some embodiments, the MVC is a trivalent radical of the organic compound. In some embodiments, the MVC is a tetravalent radical of the organic compound.

In some embodiments, Rₐ is an HTM and R_{b} is a hydrogen or a C₁₋₄ alkyl. In some embodiments, Rₐ and R_{b} are each independently an HTM.

In some embodiments, Rₐ and R_{b} combined with nitrogen to form a 3-member to 8-member heterocyclic ring substituted with an HTM. In some embodiments, the heterocyclic ring is a 5-member to 7-member ring. In some embodiments, the heterocyclic ring is a 6-member ring. In some embodiments, the heterocyclic ring is a diazinane ring. In some embodiments, the diazinane ring is a 1,4-diazinane ring. In some embodiments, the 1,4-diazinane ring is substituted with the HTM at the 4 position.

In some embodiments of the second refinement of the second aspect, the MVC is selected from:

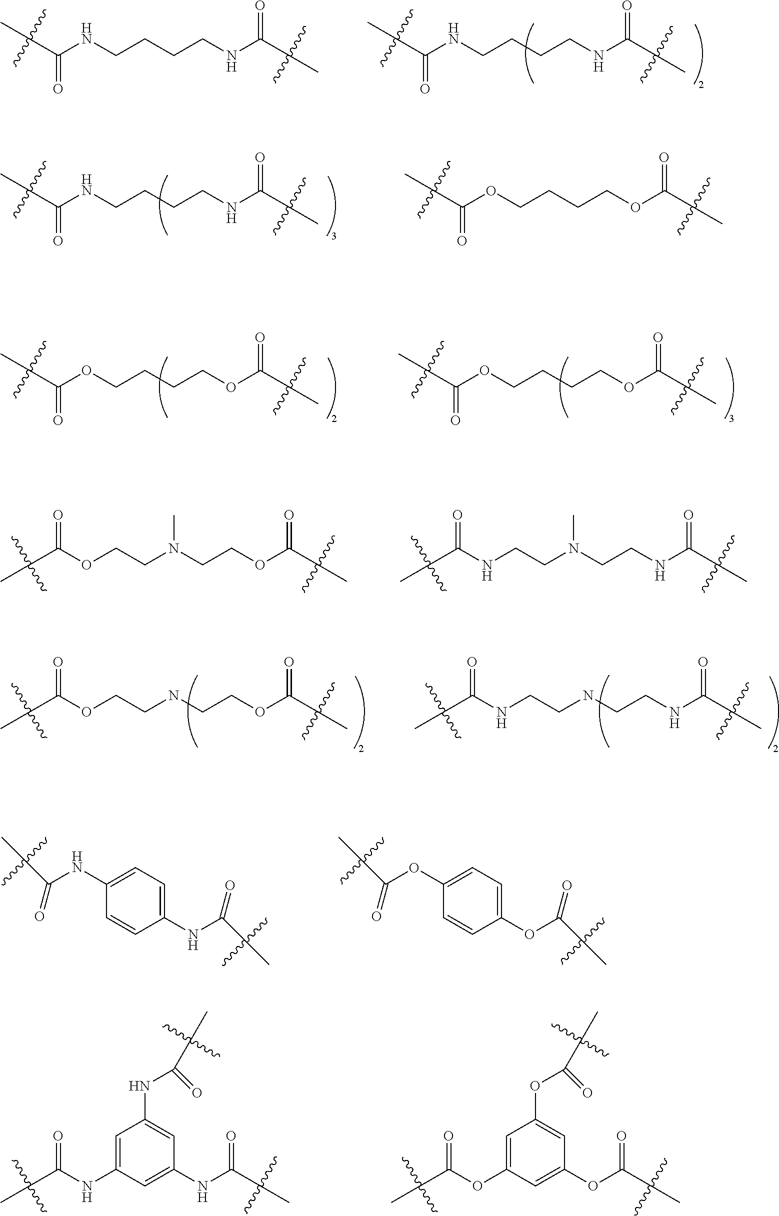

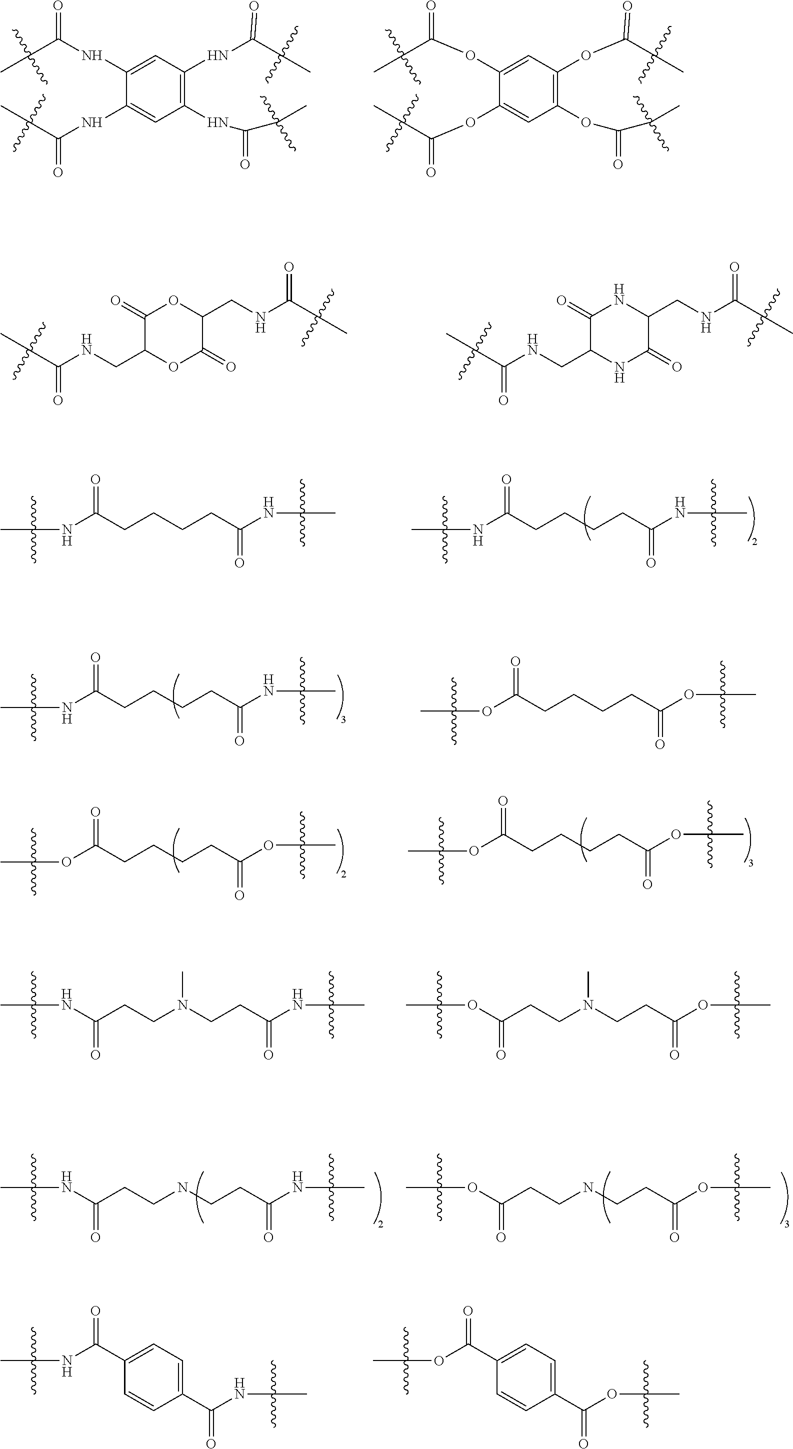

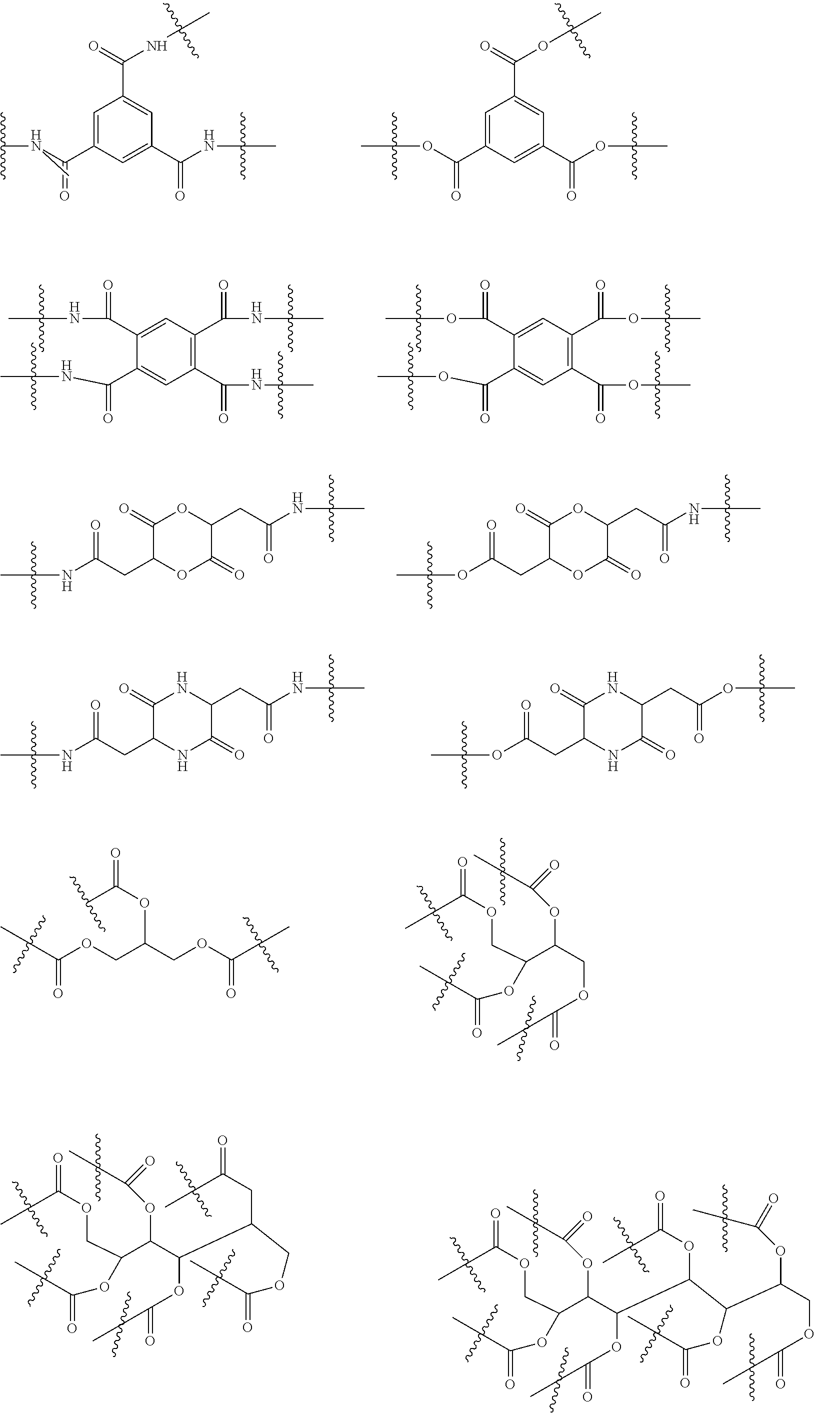

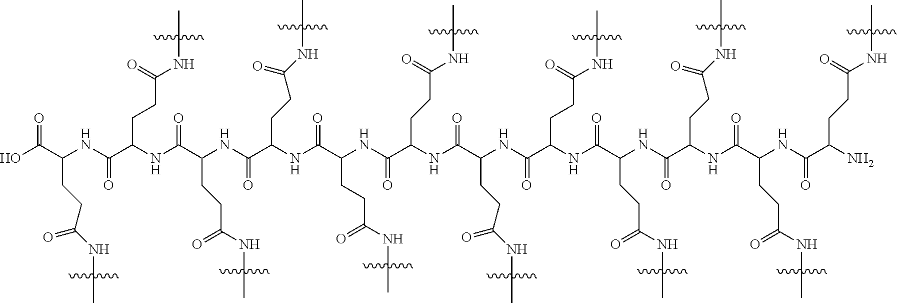 [SMILES: *NC(=O)CCC(N)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)O]

In some embodiments of the second aspect of the present disclosure, HTM is a C₅₋₄₀ linear or branched alkyl, C₅₋₄₀ linear or branched alkenyl, or C₅₋₄₀ linear or branched alkynyl.

In some embodiments, HTM is a C₅₋₄₀ linear or branched alkyl. In some embodiments, HTM is a C₅₋₃₀ linear alkyl. In some embodiments, HTM is a C₅₋₂₀ linear alkyl. In some embodiments, HTM is a C₈₋₂₀ linear alkyl. In some embodiments, HTM is a C₁₀₋₂₀ linear alkyl. In some embodiments, HTM is a C₁₂₋₂₀ linear alkyl. In some embodiments, HTM is a C₁₄₋₂₀ linear alkyl. In some embodiments, HTM is a C₁₆₋₂₀ linear alkyl. In some embodiments, HTM is a C₁₆₋₁₈ linear alkyl.

In some embodiments, HTM is a C₁₀₋₃₀ branched alkyl. In some embodiments, the branched alkyl has one C₅₋₁₅ alkyl branch on a C₅₋₁₅ alkyl main chain. In some embodiments, HTM is a C₁₅₋₄₀ branched alkyl. In some embodiments, the branched alkyl has two C₅₋₁₅ alkyl branches on a C₅₋₁₅ alkyl main chain.

In some embodiments, the linear or branched alkyl, alkenyl, or alkynyl has one or more intervening groups selected from ester —(C═O)—O—, amide —(C═O)—NR—, or carboxylate —O—(C═O)—O— wherein R is hydrogen, a C₁₋₄ alkyl, C₁₋₄ alkenyl, or C₁₋₄ alkynyl. In some embodiments, one or more intervening groups is selected from ester —(C═O)—O— or amide —(C═O)—NR—. In some embodiments, the one or more intervening group is inserted in the alkyl such that the acyl group is connect to nitrogen in Formula I or Formula II through an ethylene —CH₂—CH₂—. In some embodiments, the one or more intervening group is one or more ester group.

In some embodiments, HTM has the structure of:

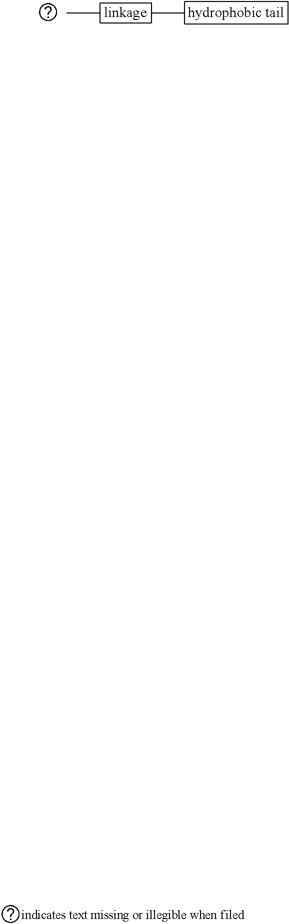

wherein the linkage is selected from

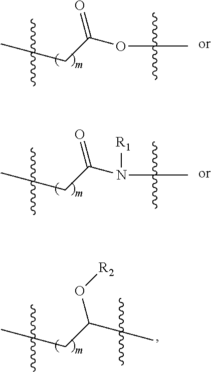

m is an integer of 1-6, R₁ is H, CH₃, or CH₂CH₃; and R₂ is H, CH₃, CH₂CH₃, or (C═O)CH₃.
wherein the hydrophobic tail is selected from the group consisting of:

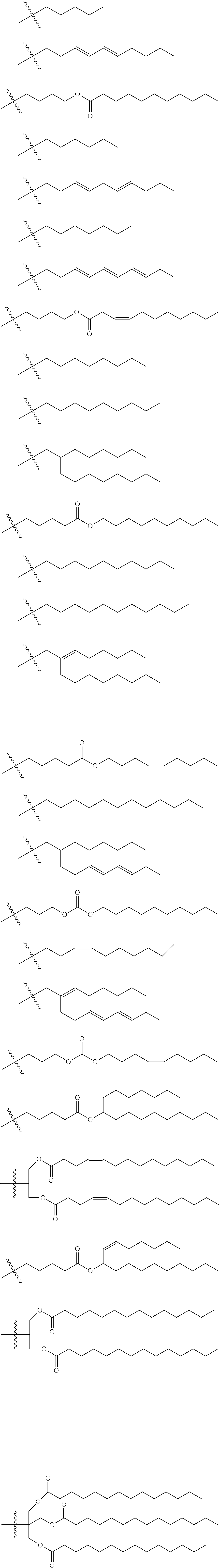

In some embodiments, the compound according to the second aspect of the present disclosure is selected from the group consisting of:

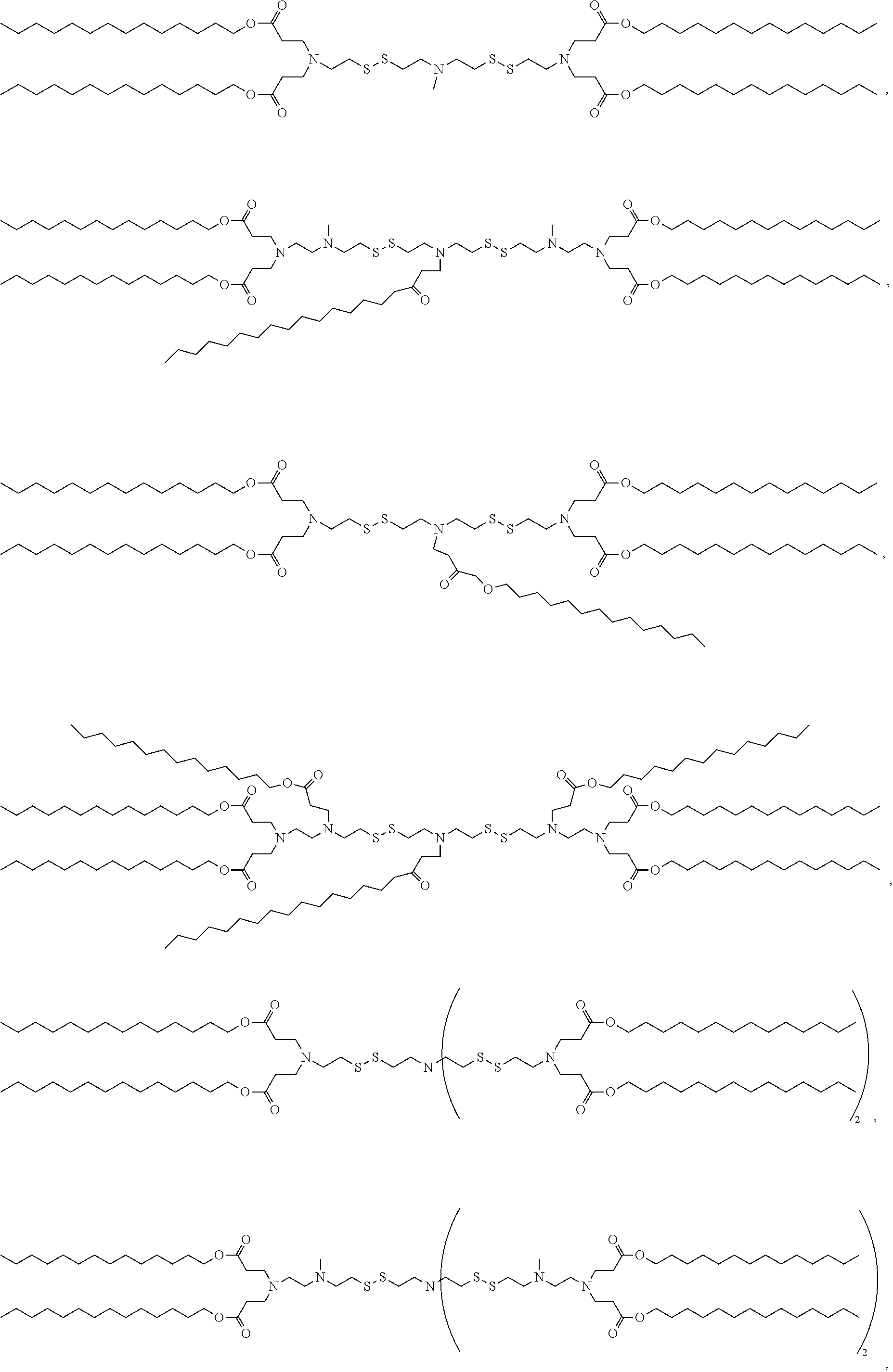

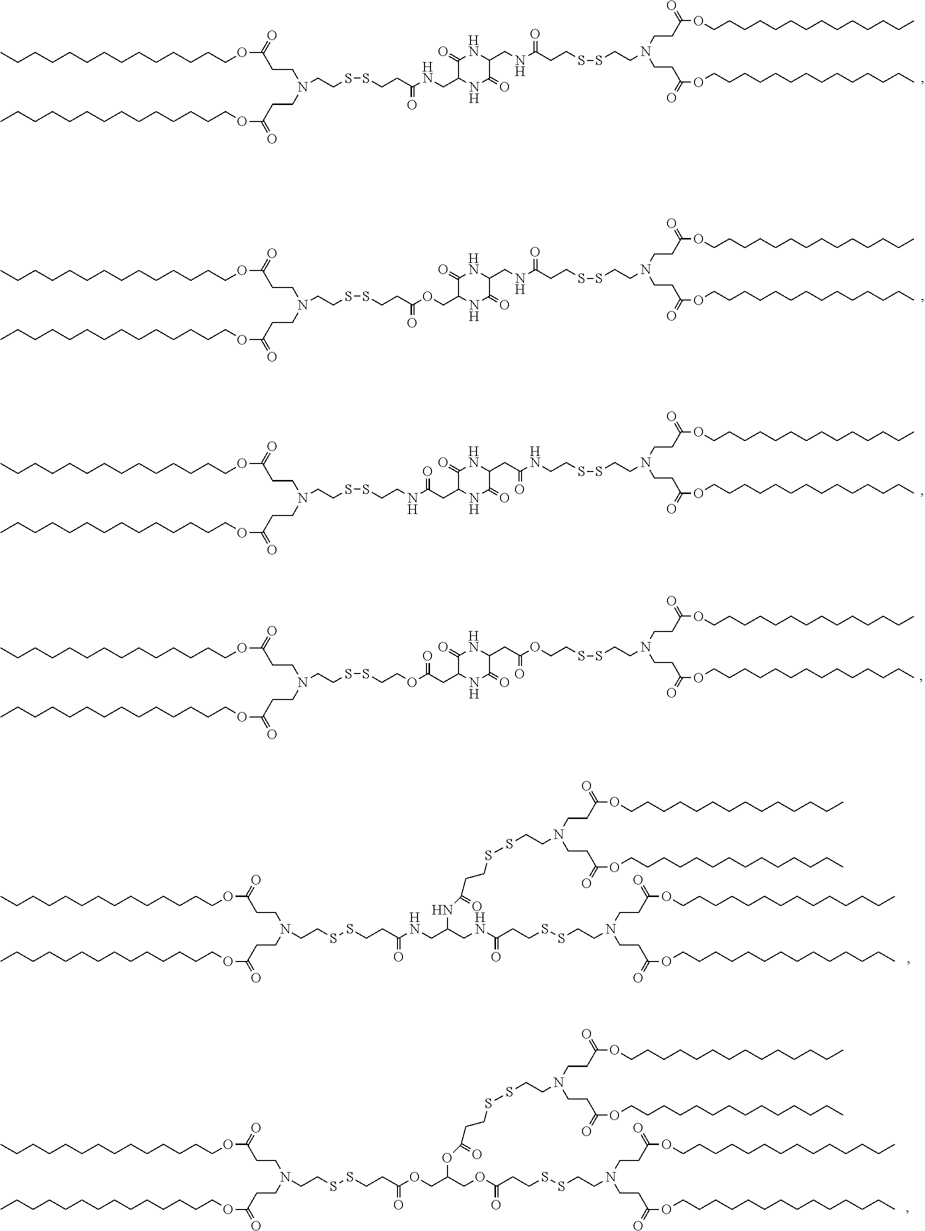

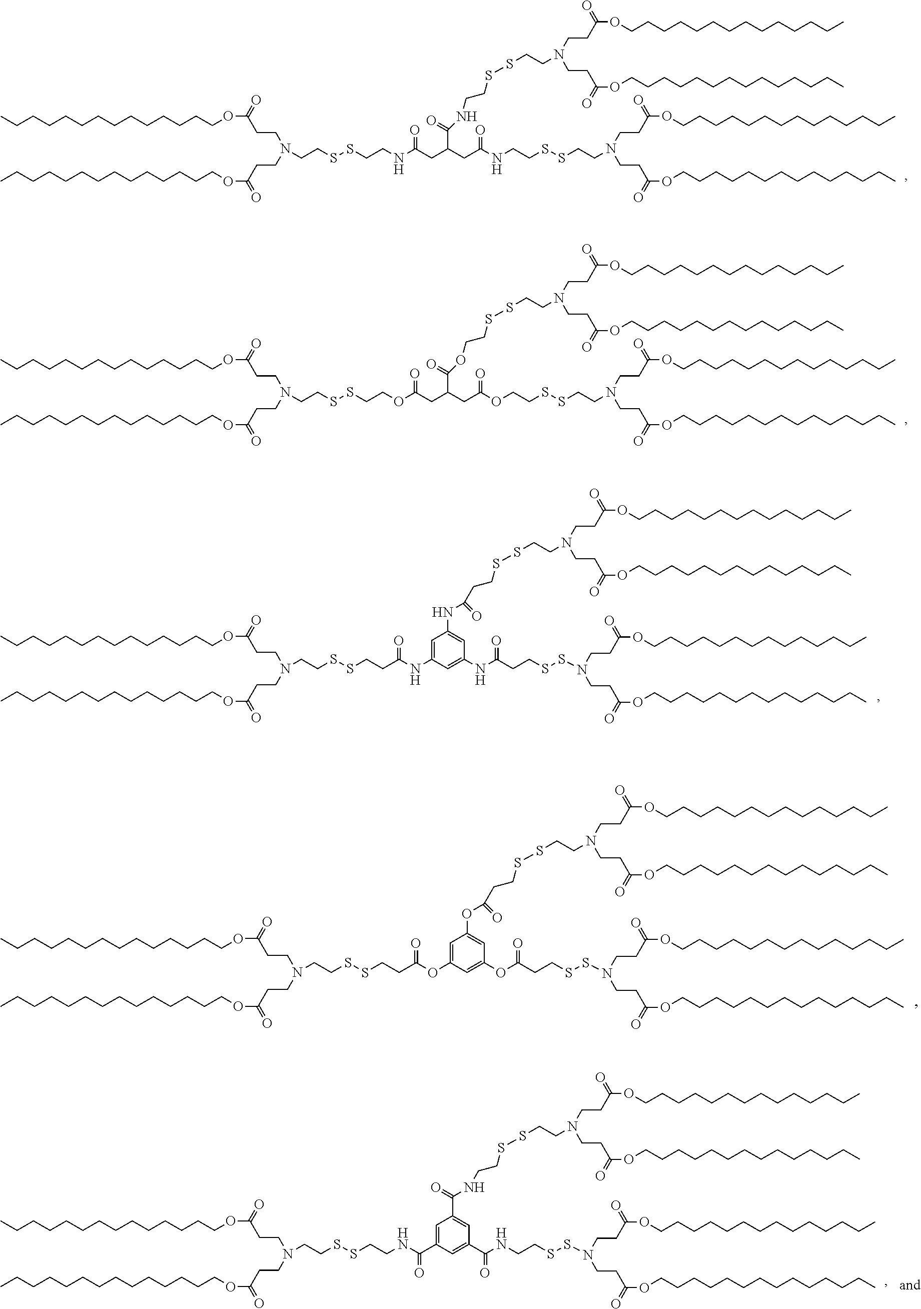

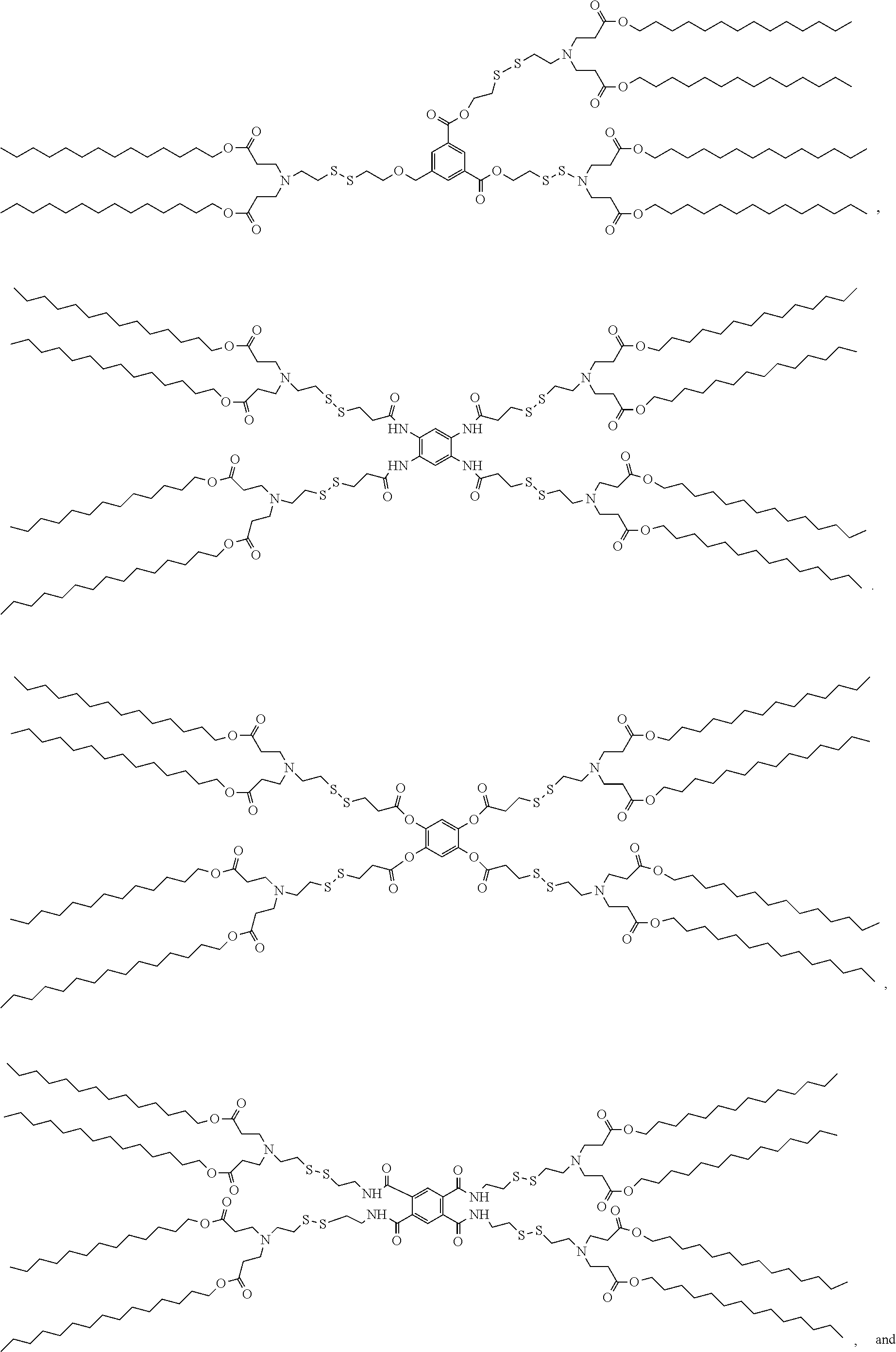

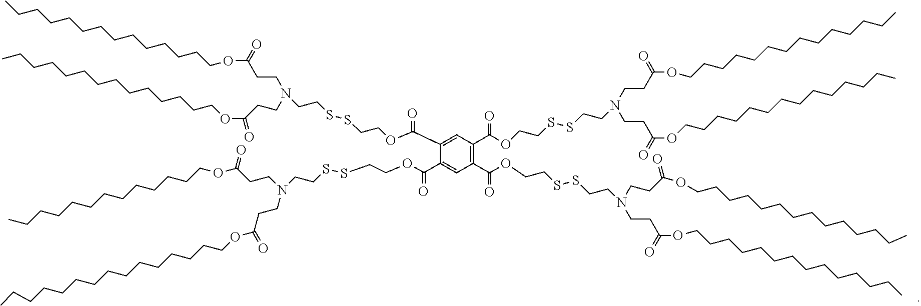 [SMILES: CCCCCCCCCCCCCCCOC(=O)CCN(CCSSCCOC(=O)c1cc(C(=O)OCCSSCCN(CCC(=O)OCCCCCCCCCCCCCC)CCC(=O)OCCCCCCCCCCCCCC)c(C(=O)OCCSSCCN(CCC(=O)OCCCCCCCCCCCCCC)CCC(=O)OCCCCCCCCCCCCCC)cc1C(=O)OCCSSCCN(CCC(=O)OCCCCCCCCCCCCCC)CCC(=O)OCCCCCCCCCCCCCC)CCC(=O)OCCCCCCCCCCCCC]

Provided in another aspect is a nanoparticle formulation comprising an active agent and any one of the compounds disclosed herein or a pharmaceutically acceptable salt thereof In some embodiment, the active agent is a nucleic acid. In some embodiments, the active agent is a large molecule. In some embodiment, the active agent is a small molecule.

Provided in another aspect is a pharmaceutical composition comprising a pharmaceutically acceptable excipient and the nanoparticle formulation disclosed herein.

A method for treating a cancer in a subject in need thereof comprising administering a therapeutically effective amount of the pharmaceutical composition disclosed herein.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a schematic representation of using the disclosed CDM compounds to package a drug, such as a nucleic acid drug, into lipid nanoparticles;

FIG. 2 illustrates a schematic representation of using the lipid nanoparticles based on the disclosed CDM compounds to deliver a drug, such as a nucleic acid drug, across cell membranes to reach an intracellular target;

FIG. 3 is a ¹HNMR spectrum of an exemplary CDM intermediate (CLLM-1);

FIG. 4 is a ¹HNMR spectrum of an exemplary Type I CDM compound (CLLM-2);

FIG. 5 is a ¹HNMR spectrum of an exemplary CDM compound/intermediate (CLLM-3);

FIG. 6 is a ¹HNMR spectrum of an exemplary Type IV CDM compound (CLLM-4);

FIG. 7 is a ¹HNMR spectrum of an exemplary CDM intermediate (CLLM-5);

FIG. 8 is a ¹HNMR spectrum of an exemplary Type IV CDM compound (CLLM-6);

FIG. 9 illustrates the Tyndell effect arises from nanoparticle formulations of CDM compounds (CLLM-2, CLLM-4, and CLLM-6) with mRNA in comparison with free mRNA without the CDM compounds;

FIG. 10 illustrates the Tyndell effect arises from nanoparticle formulations of CDM compounds (CLLM-2, CLLM-4, and CLLM-6) with pDNA in comparison with free pDNA without the CDM compounds;

FIG. 11 illustrates delivery of mRNA into 4T1 murine breast cells by nanoparticle formulations of CDM compounds (CLLM-2, CLLM-4, and CLLM-6) with mRNA in comparison with free mRNA without the CDM compounds. The drug delivery was tolerated at 100 ng mRNA in 200 uL medium per well.

FIG. 12 illustrates delivery of pDNA into 4T1 murine breast cells by nanoparticle formulations of CDM compounds (CLLM-2, CLLM-4, and CLLM-6) with pNDA in comparison with free mRNA without the CDM compounds. The drug delivery was tolerated at 200 ng pDNA in 200 uL medium per well.

### DETAILED DESCRIPTION OF THE DISCLOSURE

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, in some embodiments, ranges and amounts are expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used in the specification and appended claims, unless specified to the contrary, the following terms have the meaning indicated below.

“Amino” refers to the —NH₂radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” refers to a straight or branched hydrocarbon chain radical consisting solely of carbon and hydrogen atoms, containing no unsaturation, having one or more carbon atoms (e.g., C₁-C₁₈ alkyl). In certain embodiments, an alkyl comprises one to thirteen carbon atoms (e.g., C₁-C₁₂ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tent-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl. In addition, unless stated otherwise specifically in the specification, an alkyl group according to the present disclosure optionally include one of the following “intervening group” inserted into the carbon chain: ether, amino, carbonyl, ester, amide, or carboxylate group.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having two or more carbon atoms. In certain embodiments, an alkenyl comprises two to eighteen carbon atoms. In other embodiments, an alkenyl comprises two to fourteen carbon atoms. The alkenyl is attached to the rest of the molecule by a single bond, for example, ethenyl (i.e., vinyl), prop-1-enyl (i.e., allyl), but-l-enyl, pent-l-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl. In addition, unless stated otherwise specifically in the specification, an alkyl group according to the present disclosure optionally include one of the following “intervening group” inserted into the carbon chain: ether, amino, carbonyl, ester, amide, or carboxylate group.

“Alkynyl” refers to a straight or branched hydrocarbon chain radical group consisting solely of carbon and hydrogen atoms, containing at least one carbon-carbon triple bond, having two or more carbon atoms. In certain embodiments, an alkynyl comprises two to eighteen carbon atoms. In other embodiments, an alkynyl has two to fourteen carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl. In addition, unless stated otherwise specifically in the specification, an alkyl group according to the present disclosure optionally include one of the following “intervening group” inserted into the carbon chain: ether, amino, carbonyl, ester, amide, or carboxylate group.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. In some embodiments, the points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl. In addition, unless stated otherwise specifically in the specification, an alkenyl group according to the present disclosure optionally include one of the following “intervening group” inserted into the carbon chain: ether, amino, carbonyl, ester, amide, or carboxylate group.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Htickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—CN, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aryloxy” refers to a radical bonded through an oxygen atom of the formula —O-aryl, where aryl is as defined above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{c}-aryl, where R^{c} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, and in some embodiments, include fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. In some embodiments, the carbocyclyl is saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —CN, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O) R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” or “heterocycle” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which include fused or bridged ring systems in some embodiments. The heteroatoms in the heterocyclyl radical are optionally oxidized. One or more nitrogen atoms, if present, are optionally quaternized. The heterocyclyl radical is partially or fully saturated. In some embodiments, the heterocyclyl is attached to the rest of the molecule through any atom of the ring(s). In some embodiments, the heterocyclyl is saturated, (i.e., containing single bonds only) or unsaturated (i.e., containing one or more double bonds or triple bonds.) A fully saturated heterocyclyl radical is also referred to as “heterocycloalkyl.” Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —CN, —R^{b}—CN, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R b is independently a direct bond or a straight or branched alkylene or alkenylene chain, and RC is a straight or branched alkylene or alkenylene chain, and where each of the above substituents is unsubstituted unless otherwise indicated.

“Heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl. In some embodiments, the alkyl part of the heteroalkyl radical is optionally substituted as defined for an alkyl group.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O-R° -heterocyclyl where RC is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, in some embodiments, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Htickel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S)O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O-heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, the compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para- isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

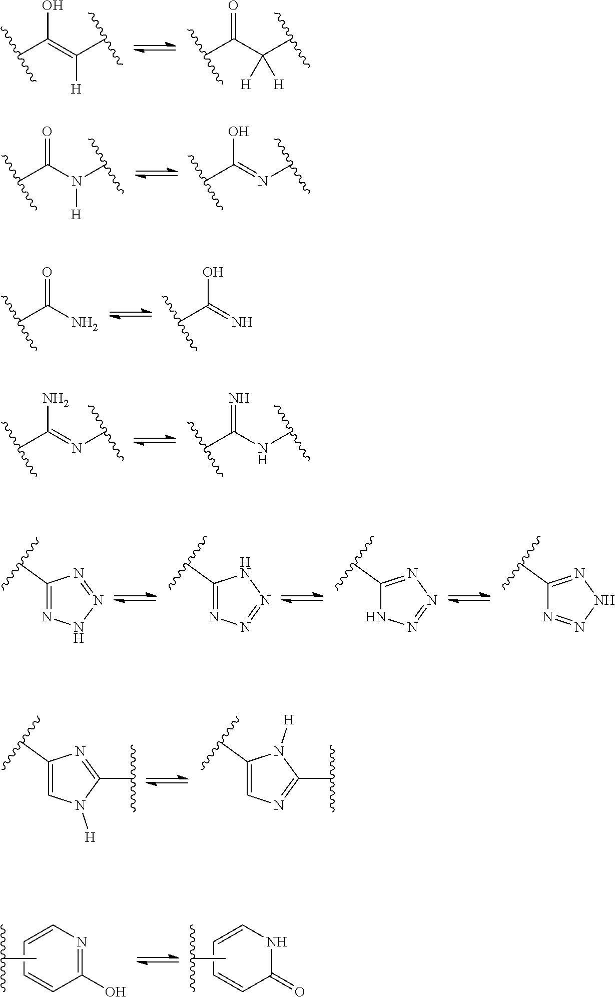

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66: 1-19 (1997), which is hereby incorporated by reference in its entirety). In some embodiments, acid addition salts of basic compounds are prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating ” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is afflicted with the underlying disorder in some embodiments. For prophylactic benefit, in some embodiments, the compositions are administered to a patient at risk of developing a particular disease, or to a patient reporting one or more of the physiological symptoms of a disease, even though a diagnosis of this disease has not been made.

“Prodrug” is meant to indicate a compound that is converted under physiological conditions or by solvolysis to a biologically active compound described herein. Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. In some embodiments, prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

### CDM Compounds

The disclosure relates to compounds, synthetic methods, characterization, and uses of cleavable disulfide molecules (CDMs). Some CDM compounds according to the present disclosure have one disulfide bond (—S—S—). Some CDM compounds according to the present disclosure have multiple disulfide bonds. The CDM compounds have one or more hydrophobic tail moieties (HTMs). The chemical structures of those compounds are summarized herein as non-limiting embodiments.

### Single-Disulfide CDM Compounds

A first aspect of the present disclosure relates to a compound of Formula I, a stereoisomer or pharmaceutically acceptable salt thereof:

RₐR_{b}N-L₁-S—S-L₂-NR_{c}R_{d} (Formula I)
wherein L₁ and L₂ are each independently C₁₋₄ alkylenyl;
wherein at least one of Rₐ, R_{b}, R_{c} and R_{d} is independently a hydrophobic tail moiety (HTM) or combined with nitrogen on the same side of Formula I to form a 3-member to 8-member heterocyclic ring substituted with an HTM, and the wherein the remainder of Rₐ, R_{b}, R_{c} and R_{d} is independently a hydrogen, a C₁₋₄ alkyl, or combine with nitrogen on the same side of Formula I to form a 3-member to 8-member heterocyclic ring optionally substituted with a C₁₋₄ alkyl,
wherein HTM is a C₅₋₄₀ linear or branched alkyl, C₅₋₄₀ linear or branched alkenyl, or C₅₋₄₀ linear or branched alkynyl; or wherein HTM has a structure of Formula II:

-L-NR′R″ (Formula II)

wherein L is C₁₋₄ alkylenyl and at least one of R′ and R″ is a C₅₋₄₅ linear or branched alkyl, C₅₋₄₅ linear or branched alkenyl, or C₅₋₄₅ linear or branched alkynyl.

In some embodiments, L1 and L2 are each independently C1-3 alkylenyl. In some embodiments, L1 is ethylene. In some embodiments, L2 is ethylene.

### Type I CDM Compounds

In a refinement of the first aspect, Rₐ and R_{b} is independently a hydrogen, a C₁₋₄ alkyl, or combine with nitrogen on the same side of Formula I to form a 3-member to 8-member heterocyclic ring optionally substituted with a C₁₋₄ alkyl.

In some embodiments, Rₐ and R_{b} are hydrogen.

In some embodiments, the Rₐ is hydrogen and R_{b} is a C₁₋₄ alkyl. In some embodiments, R_{b} is a butyl.

In some embodiments, Rₐ and R_{b} are each independently a C₁₋₄ alkyl. In some embodiments, Rₐ and R_{b} are methyl.

In some embodiments, Rₐ and R_{b} combine with nitrogen on the same side of Formula I to form a 3-member to 8-member heterocyclic ring. In some embodiments, the heterocyclic ring is substituted with a C₁₋₄ alkyl.

In some embodiments, the heterocyclic ring is a 5-member to 7-member ring. In some embodiments, the heterocyclic ring is a 6-member ring. In some embodiments, the heterocyclic ring is a diazinane ring. In some embodiments, the diazinane ring is a 1,4-diazinane ring. In some embodiments, the 1,4-diazinane ring is substituted with a C₁₋₄ alkyl at the 4 position.

In some embodiments, R_{c} is an HTM and R_{d} is a hydrogen or a C₁₋₄ alkyl. In some embodiments, R_{c} and R_{d} are each independently an HTM.

In some embodiments, R_{c} and R_{d} combined with nitrogen on the same side of Formula I to form a 3-member to 8-member heterocyclic ring substituted with an HTM. In some embodiments, the heterocyclic ring is a 5-member to 7-member ring. In some embodiments, the heterocyclic ring is a 6-member ring. In some embodiments, the heterocyclic ring is a diazinane ring. In some embodiments, the diazinane ring is a 1,4-diazinane ring. In some embodiments, the 1,4-diazinane ring is substituted with the HTM at the 4 position.

### Type II CDM Compounds

In another refinement of the first aspect, at least one of Rₐ and R_{b} is an HTM or Rₐ and R_{b} combine with nitrogen on the same side of Formula one to form a 3-member to 8-member heterocyclic ring substituted with an HTM; and at least one of R_{c} and R_{d} is an HTM or R_{c} and R_{d} combine with nitrogen on the same side of Formula one to form a 3-member to 8-member heterocyclic ring substituted with an HTM.

In some embodiments, at least one of Rₐ and R_{b} is an HTM, and wherein at least one of R_{c} and R_{d} is an HTM.

In some embodiments, Rₐ is an HTM and R_{b} is hydrogen or a C₁₋₄ alkyl. In some embodiments, R_{b} is hydrogen. In some embodiments, R_{b} is a C₁₋₄ alkyl.

In some embodiments, R_{c} is an HTM and R_{d} is hydrogen or a C₁₋₄ alkyl. In some embodiments, R_{d} is hydrogen. In some embodiments, R_{d} is a C₁₋₄ alkyl.

In some embodiments, the C₁₋₄ alkyl is methyl. In some embodiments, the C₁₋₄ alkyl is butyl.

In some embodiments, R_{c} and R_{d} are each independently an HTM. In some embodiments, Rₐ, R_{b}, R_{c} and R_{d} are each independently an HTM.

In some embodiments, at least one of Rₐ and R_{b} is an HTM, and R_{c} and R_{d} combine with nitrogen on the same side of Formula one to form a 3-member to 8-member heterocyclic ring substituted with an HTM.

In some embodiments, Rₐ is an HTM and R_{b} is hydrogen or a C₁₋₄ alkyl. In some embodiments, R_{b} is hydrogen. In some embodiments, R_{b} is a C₁₋₄ alkyl. In some embodiments, the C₁₋₄ alkyl is methyl. In some embodiments, the C₁₋₄ alkyl is butyl.

In some embodiments, Rₐ and R_{b} are each independently an HTM.

In some embodiments, Rₐ and R_{b} combine with nitrogen on the same side of Formula one to form a 3-member to 8-member first heterocyclic ring substituted with an HTM; and R_{c} and R_{d} combine with nitrogen on the same side of Formula one to form a second 3-member to 8-member heterocyclic ring substituted with an HTM.

In some embodiments, the first and second heterocyclic rings are each independently a 5-member to 7-member ring. In some embodiments, the first and second heterocyclic rings are each independently a 6-member ring. In some embodiments, the first and second heterocyclic rings are each independently a diazinane ring. In some embodiments, the diazinane ring is a 1,4-diazinane ring. In some embodiments, the 1,4-diazinane ring is substituted with the HTM at the 4 position.

In some embodiments, the single disulfide CDM compounds disclosed herein have the structure provided in Table 1.

TABLE 1

| | | CDM |
|---|---|---|
| No. | Structure | Type |
| 1 | 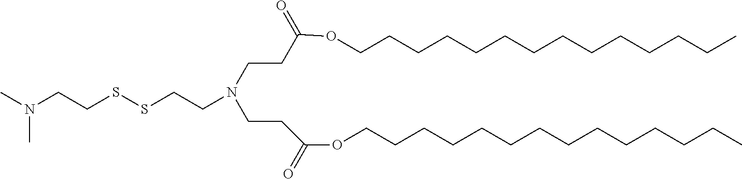 [SMILES: CCCCCCCCCCCCCCOC(=O)CCN(CCSSCCN(C)C)CCC(=O)OCCCCCCCCCCCCCC] | I |
| 2 | 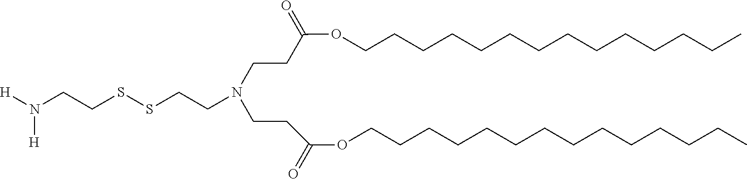 [SMILES: CCCCCCCCCCCCCCOC(=O)CCN(CCSSCCN)CCC(=O)OCCCCCCCCCCCCCC] | I |
| 3 | 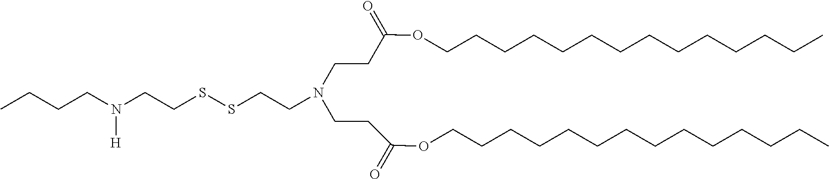 [SMILES: CCCCCCCCCCCCCCOC(=O)CCN(CCSSCCNCCCC)CCC(=O)OCCCCCCCCCCCCCC] | I |
| 4 | 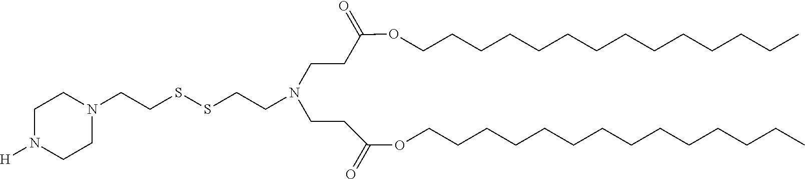 [SMILES: CCCCCCCCCCCCCCOC(=O)CCN(CCSSCCN1CCNCC1)CCC(=O)OCCCCCCCCCCCCCC] | I |
| 5 | 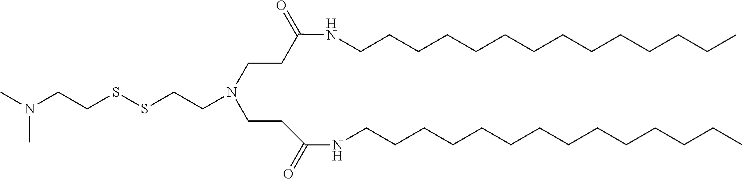 [SMILES: CCCCCCCCCCCCCCNC(=O)CCN(CCSSCCN(C)C)CCC(=O)NCCCCCCCCCCCCCC] | I |
| 6 | 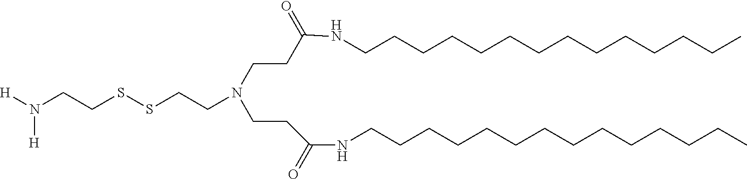 [SMILES: CCCCCCCCCCCCCCNC(=O)CCN(CCSSCCN)CCC(=O)NCCCCCCCCCCCCCC] | I |
| 7 | 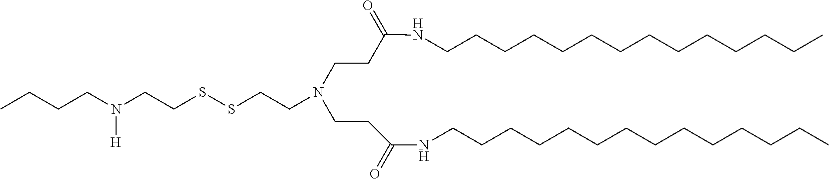 [SMILES: CCCCCCCCCCCCCCNC(=O)CCN(CCSSCCNCCCC)CCC(=O)NCCCCCCCCCCCCCC] | I |
| 8 | 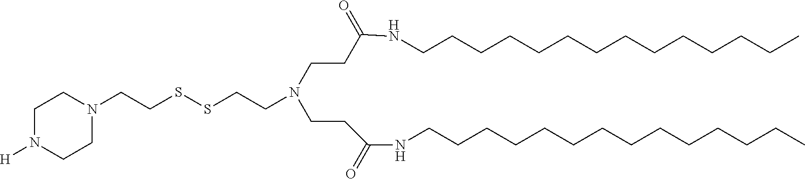 [SMILES: CCCCCCCCCCCCCCNC(=O)CCN(CCSSCCN1CCNCC1)CCC(=O)NCCCCCCCCCCCCCC] | I |
| 9 | 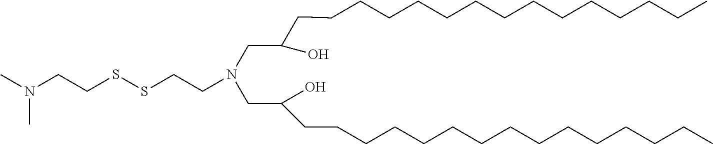 [SMILES: CCCCCCCCCCCCCCCC(O)CN(CCSSCCN(C)C)CC(O)CCCCCCCCCCCCCCC] | I |
| 10 | 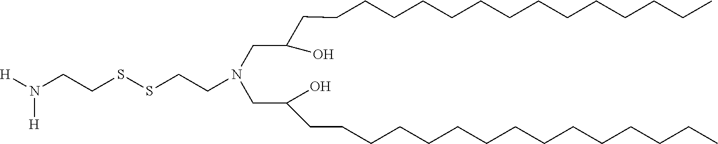 [SMILES: CCCCCCCCCCCCCCCC(O)CN(CCSSCCN)CC(O)CCCCCCCCCCCCCCC] | I |
| 11 | 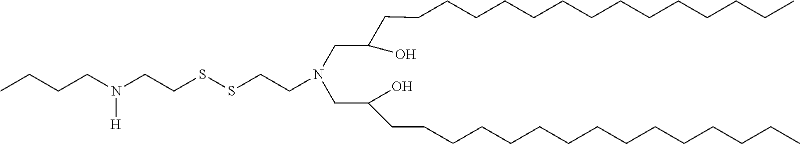 [SMILES: CCCCCCCCCCCCCCCC(O)CN(CCSSCCNCCCC)CC(O)CCCCCCCCCCCCCCC] | I |
| 12 | 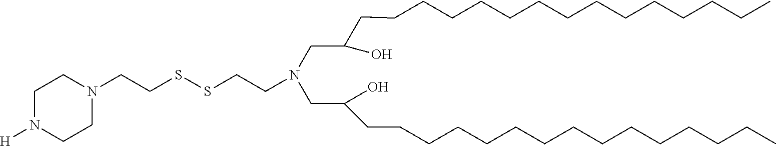 [SMILES: CCCCCCCCCCCCCCCC(O)CN(CCSSCCN1CCNCC1)CC(O)CCCCCCCCCCCCCCC] | I |
| 13 | 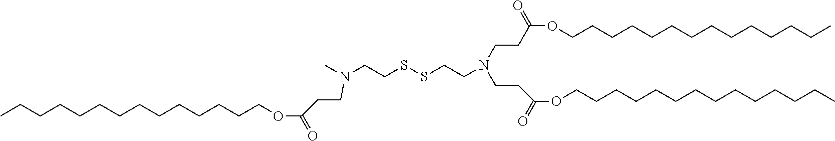 [SMILES: CCCCCCCCCCCCCCOC(=O)CCN(C)CCSSCCN(CCC(=O)OCCCCCCCCCCCCCC)CCC(=O)OCCCCCCCCCCCCCC] | II |
| 14 | 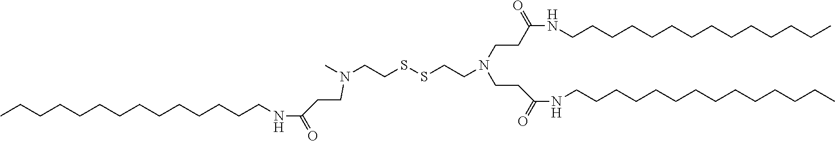 [SMILES: CCCCCCCCCCCCCCNC(=O)CCN(C)CCSSCCN(CCC(=O)NCCCCCCCCCCCCCC)CCC(=O)NCCCCCCCCCCCCCC] | II |
| 15 | 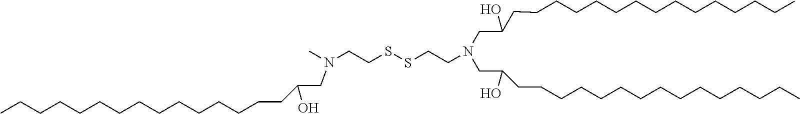 [SMILES: CCCCCCCCCCCCCCCC(O)CN(C)CCSSCCN(CC(O)CCCCCCCCCCCCCCC)CC(O)CCCCCCCCCCCCCCC] | II |
| 16 | 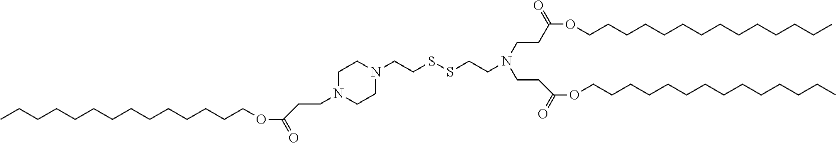 [SMILES: CCCCCCCCCCCCCCOC(=O)CCN(CCSSCCN1CCN(CCC(=O)OCCCCCCCCCCCCCC)CC1)CCC(=O)OCCCCCCCCCCCCCC] | II |
| 17 | 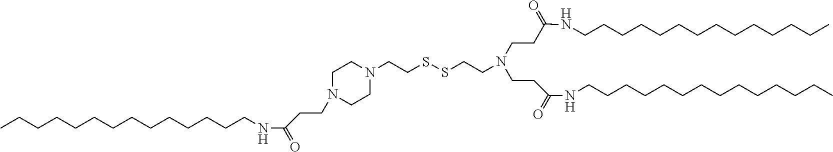 [SMILES: CCCCCCCCCCCCCCNC(=O)CCN(CCSSCCN1CCN(CCC(=O)NCCCCCCCCCCCCCC)CC1)CCC(=O)NCCCCCCCCCCCCCC] | II |
| 18 | 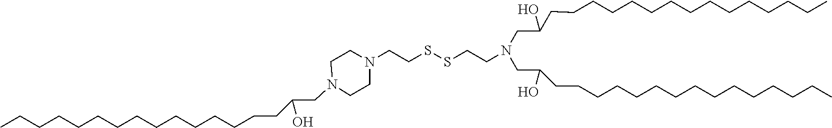 [SMILES: CCCCCCCCCCCCCCCC(O)CN1CCN(CCSSCCN(CC(O)CCCCCCCCCCCCCCC)CC(O)CCCCCCCCCCCCCCC)CC1] | II |
| 19 | 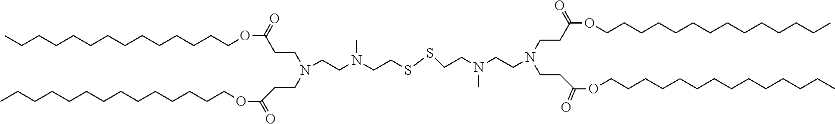 [SMILES: CCCCCCCCCCCCCCOC(=O)CCN(CCC(=O)OCCCCCCCCCCCCCC)CCN(C)CCSSCCN(C)CCN(CCC(=O)OCCCCCCCCCCCCCC)CCC(=O)OCCCCCCCCCCCCCC] | II |
| 20 | 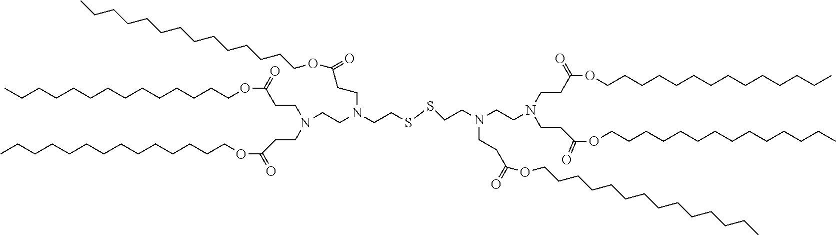 [SMILES: CCCCCCCCCCCCCCOC(=O)CCN(CCSSCCN(CCC(=O)OCCCCCCCCCCCCCC)CCN(CCC(=O)OCCCCCCCCCCCCCC)CCC(=O)OCCCCCCCCCCCCCC)CCN(CCC(=O)OCCCCCCCCCCCCCC)CCC(=O)OCCCCCCCCCCCCCC] | II |

### Multi-Disulfides CDM Compounds

A second aspect of the present disclosure relates to a compound of Formula III, a stereoisomer or pharmaceutically acceptable salt thereof:

(RₐR_{b}N-L₁-S—S-L₂-)nMVC (Formula III)
wherein MVC is a multi-valent core capable of connecting with two or more (RₐR_{b}N-L₁-S—S-L₂-), wherein n is an integer of 2-12;
wherein L₁ and L₂ are each independently C₁₋₄ alkylenyl;
wherein at least one of Rₐ and R_{b} is independently a hydrophobic tail moiety (HTM) or Rₐ and R_{b} combined with nitrogen to form a 3-member to 8-member heterocyclic ring substituted with an HTM, and the wherein the remainder of Rₐ, R_{b}, R_{c} and R_{d} is independently a hydrogen, a C₁₋₄ alkyl, or combine with nitrogen on the same side of Formula I to form a 3-member to 8-member heterocyclic ring optionally substituted with a C₁₋₄ alkyl,
wherein HTM is a C₅₋₄₀ linear or branched alkyl, C₅₋₄₀ linear or branched alkenyl, or C₅₋₄₀ linear or branched alkynyl; or wherein HTM has a structure of Formula II:

-L-NR′R″ (Formula II)

wherein L is C₁₋₄ alkylenyl and at least one of R′ and R″ is a C₅₋₄₅ linear or branched alkyl, C₅₋₄₅ linear or branched alkenyl, or C₅₋₄₅ linear or branched alkynyl.

In some embodiments, L₁ and L₂ are each independently C1-3 alkylenyl. In some embodiments, L₁ is ethylene. In some embodiments, L₂ is ethylene.

### Type III CDM Compounds

In a first refinement of the second aspect of the present disclosure, MVC is a multivalent non-metal element. In some embodiments, oxygen or nitrogen. In some embodiments, the non-metal element is nitrogen.

In some embodiments, n is 2 and wherein the MVC is a divalent nitrogen —NR₁—, wherein R₁ is a hydrogen, a C1-4 alkyl, or an HTM.

In some embodiments, the R₁ is a C₁₋₄ alkyl. In some embodiments, the R₁ is a methyl. In some embodiments, the R₁ is HTM.

In some embodiments, n is 3 and wherein the MVC is a trivalent nitrogen.

In some embodiments, Rₐ is an HTM and R_{b} is a hydrogen or a C₁₋₄ alkyl. In some embodiments, Rₐ and R_{b} are each independently an HTM.

In some embodiments, Rₐ and R_{b} combined with nitrogen to form a 3-member to 8-member heterocyclic ring substituted with an HTM. In some embodiments, the heterocyclic ring is a 5-member to 7-member ring. In some embodiments, the heterocyclic ring is a 6-member ring. In some embodiments, the heterocyclic ring is a diazinane ring. In some embodiments, the diazinane ring is a 1,4-diazinane ring. In some embodiments, the 1,4-diazinane ring is substituted with the HTM at the 4 position.

### Type IV CDM Compounds

In a second refinement of the second aspect of the present disclosure, the MVC is a multivalent radical of an organic compound having multiple functional groups. In some embodiments, the functional groups are selected from —C(═O)—O—, —O—C(═O)—, —C(═O)—NR2—, —NR2—C(═O)—, or combinations thereof.

In some embodiments, the functional groups are —C(═O)—O—. In some embodiments, the functional groups are —O—C(═O)—. In some embodiments, the functional groups are —C(═O)—NR2—. In some embodiments, the functional groups are —NR2—C(═O)—.

In some embodiments, the organic compound is a nonaromatic compound. In some embodiments, the nonaromatic compound is an aliphatic compound. In some embodiments, the nonaromatic compound is a cyclic compound. In some embodiments, the cyclic nonaromatic compound is a heterocyclic compound. In some embodiments, the cyclic nonaromatic compound is a homocyclic compound. In some embodiments, the organic compound is a C₃₋₁₅ nonaromatic compound.

In some embodiments, the organic compound is an aromatic compound. In some embodiments, the aromatic compound is an homoaromatic compound. In some embodiments, the aromatic compound is a heteroaromatic compound. In some embodiments, the aromatic compound is a heterocyclic compound. In some embodiments, the aromatic compound is a monocyclic aromatic compound. In some embodiments, the aromatic compound is a polycyclic aromatic compound. In some embodiments, the organic compound is a C₅₋₁₅ aromatic compound.

In some embodiments, the MVC is a divalent radical of the organic compound. In some embodiments, the MVC is a trivalent radical of the organic compound. In some embodiments, the MVC is a tetravalent radical of the organic compound.

In some embodiments, Rₐ is an HTM and R_{b} is a hydrogen or a C₁₋₄ alkyl. In some embodiments, Rₐ and R_{b} are each independently an HTM.

In some embodiments, Rₐ and R_{b} combined with nitrogen to form a 3-member to 8-member heterocyclic ring substituted with an HTM. In some embodiments, the heterocyclic ring is a 5-member to 7-member ring. In some embodiments, the heterocyclic ring is a 6-member ring. In some embodiments, the heterocyclic ring is a diazinane ring. In some embodiments, the diazinane ring is a 1,4-diazinane ring. In some embodiments, the 1,4-diazinane ring is substituted with the HTM at the 4 position.

In some embodiments of the second refinement of the second aspect, the MVC is selected from:

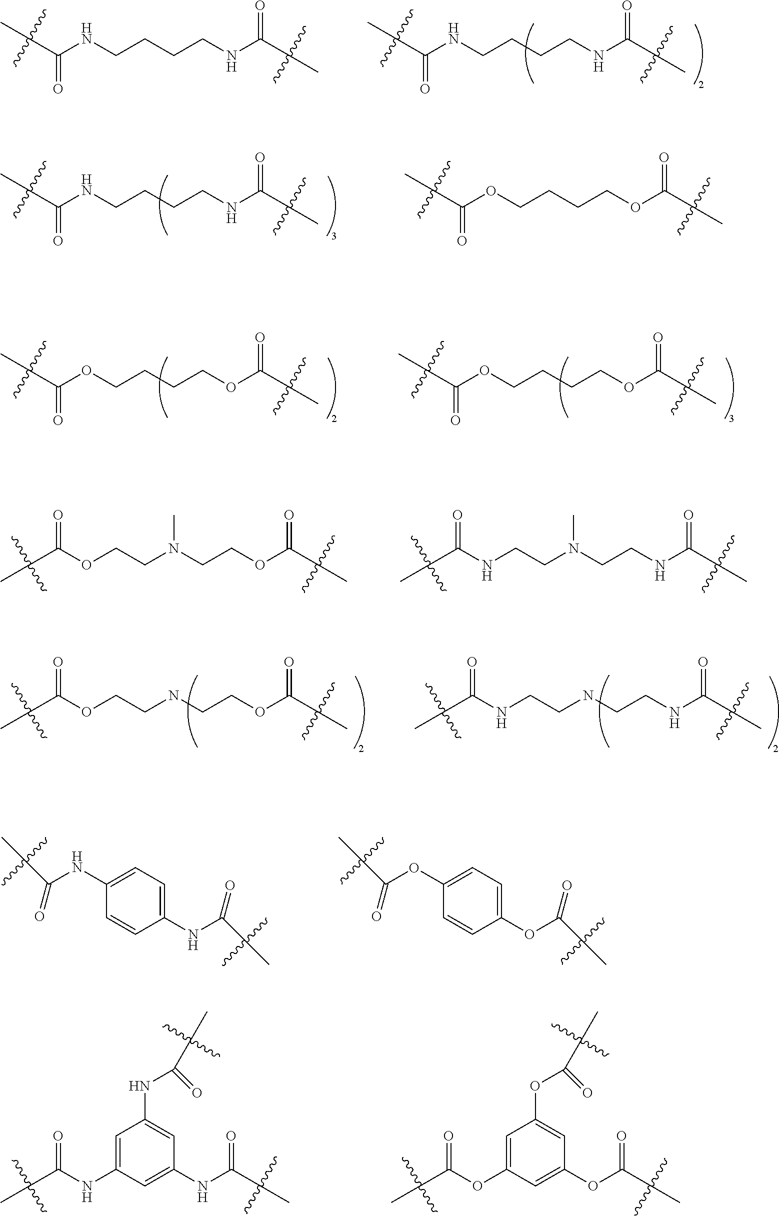

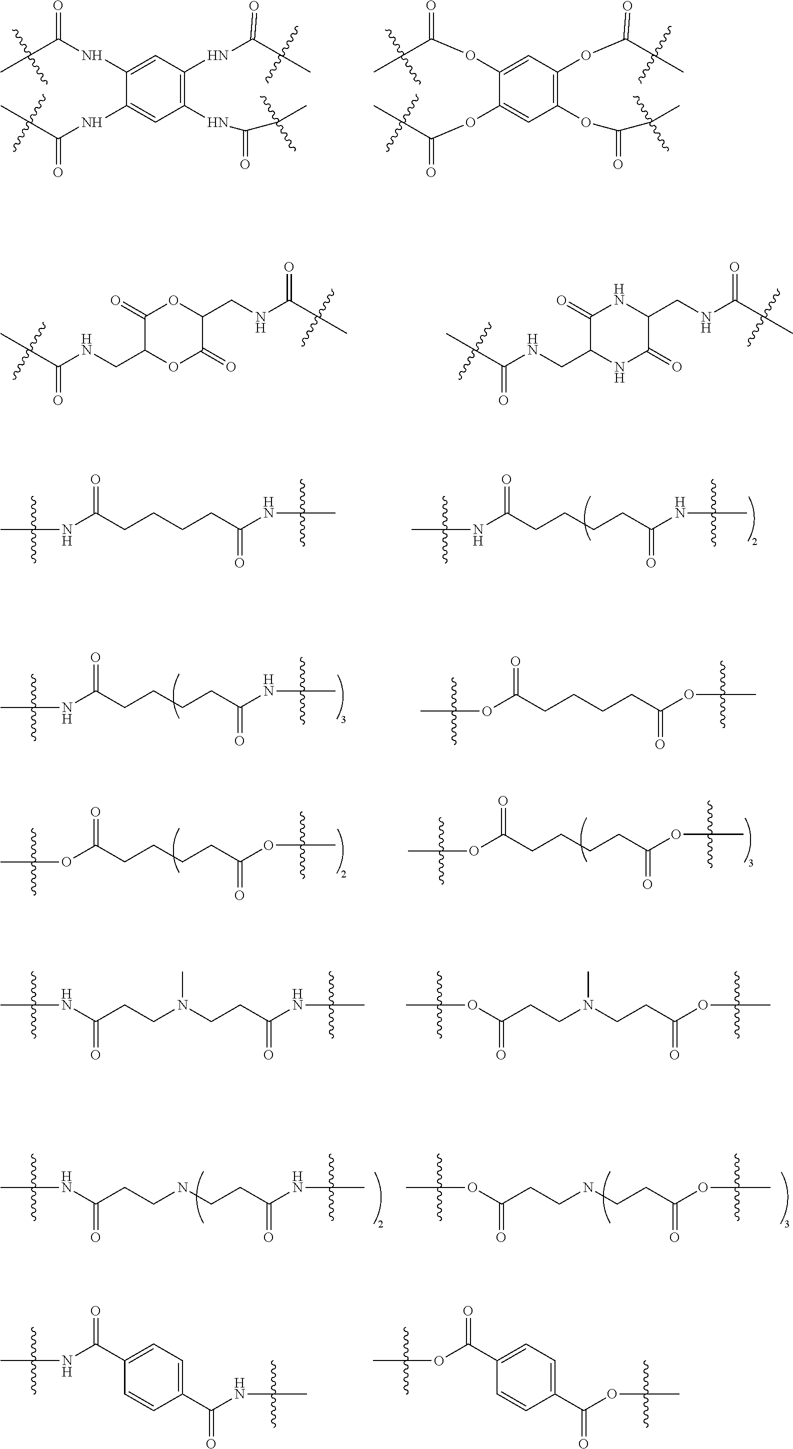

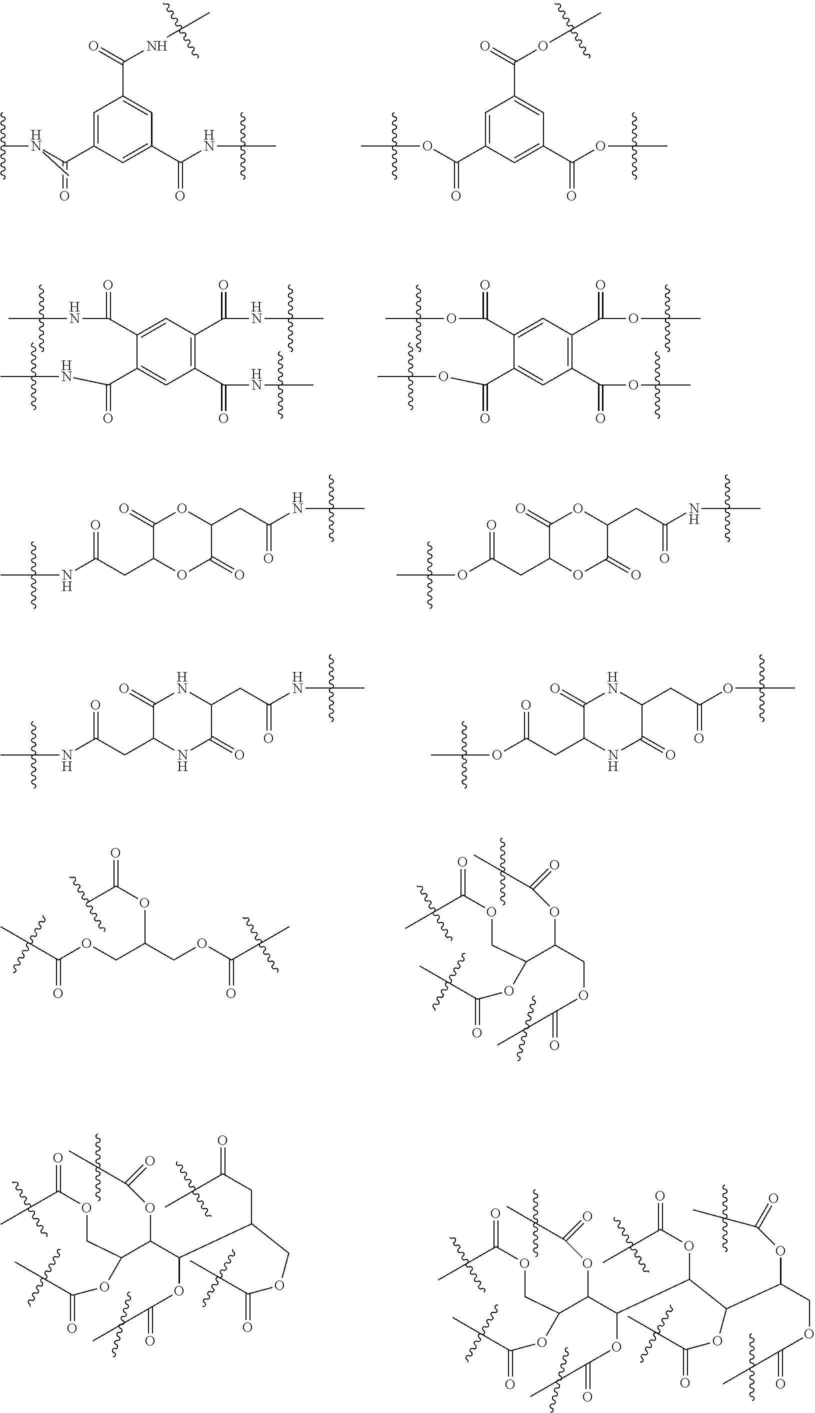

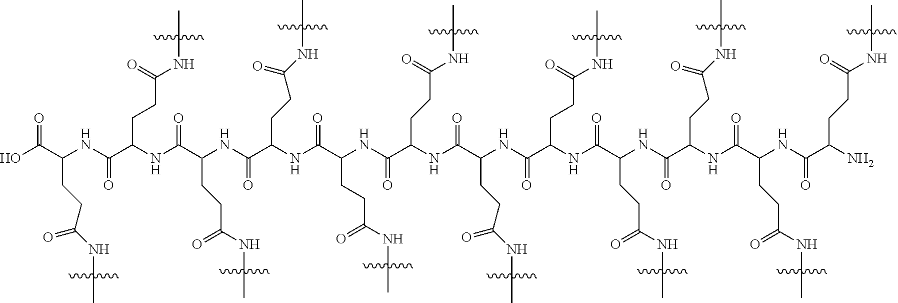 [SMILES: *NC(=O)CCC(N)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)O]

In some embodiments, the multiple disulfides CDM compounds disclosed herein have the structure provided in Table 2.

TABLE 2

| | | CDM |
|---|---|---|
| No. | Structure | Type |
| 21 | 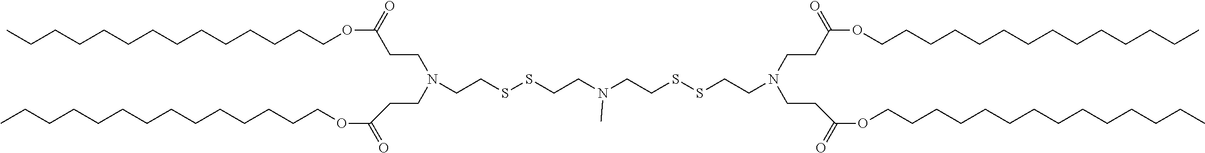 [SMILES: CCCCCCCCCCCCCCOC(=O)CCN(CCSSCCN(C)CCSSCCN(CCC(=O)OCCCCCCCCCCCCCC)CCC(=O)OCCCCCCCCCCCCCC)CCC(=O)OCCCCCCCCCCCCCC] | III |
| 22 | 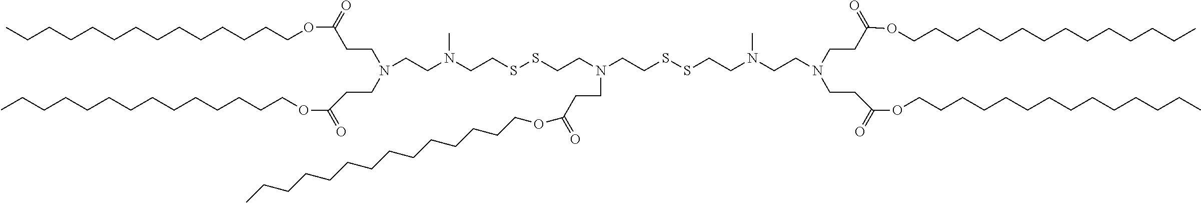 [SMILES: CCCCCCCCCCCCCCOC(=O)CCN(CCSSCCN(C)CCN(CCC(=O)OCCCCCCCCCCCCCC)CCC(=O)OCCCCCCCCCCCCCC)CCSSCCN(C)CCN(CCC(=O)OCCCCCCCCCCCCCC)CCC(=O)OCCCCCCCCCCCCCC] | III |
| 23 | 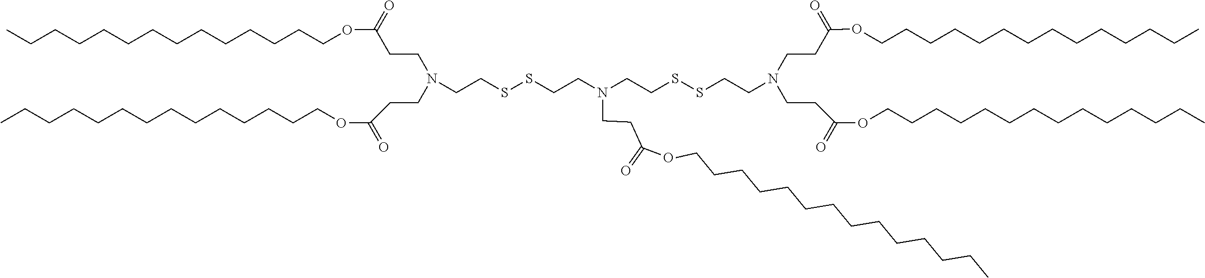 [SMILES: CCCCCCCCCCCCCCOC(=O)CCN(CCSSCCN(CCC(=O)OCCCCCCCCCCCCCC)CCC(=O)OCCCCCCCCCCCCCC)CCSSCCN(CCC(=O)OCCCCCCCCCCCCCC)CCC(=O)OCCCCCCCCCCCCCC] | III |
| 24 | 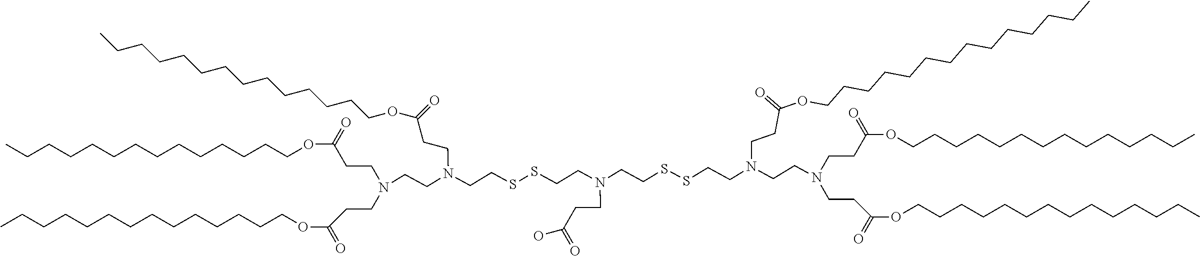 [SMILES: CCCCCCCCCCCCCCOC(=O)CCN(CCSSCCN(CCSSCCN(CCC(=O)OCCCCCCCCCCCCCC)CCN(CCC(=O)OCCCCCCCCCCCCCC)CCC(=O)OCCCCCCCCCCCCCC)CCC(=O)O)CCN(CCC(=O)OCCCCCCCCCCCCCC)CCC(=O)OCCCCCCCCCCCCCC] | III |
| 25 | 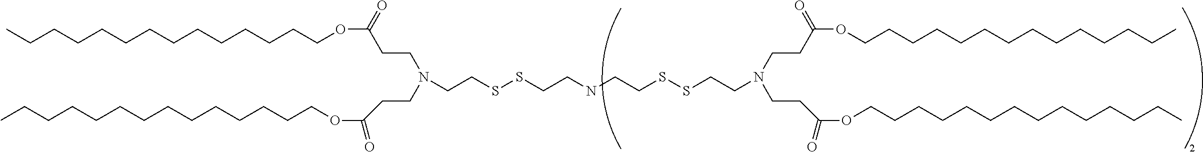 | III |
| 26 | 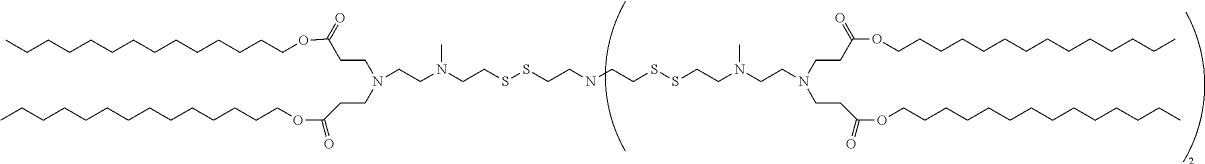 | III |
| 27 | 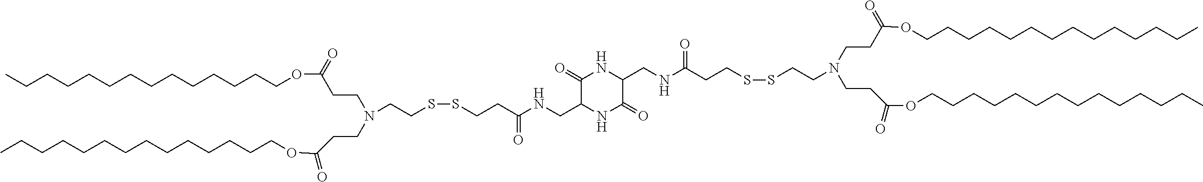 [SMILES: CCCCCCCCCCCCCCOC(=O)CCN(CCSSCCC(=O)NCC1NC(=O)C(CNC(=O)CCSSCCN(CCC(=O)OCCCCCCCCCCCCCC)CCC(=O)OCCCCCCCCCCCCCC)NC1=O)CCC(=O)OCCCCCCCCCCCCCC] | IV |
| 28 | 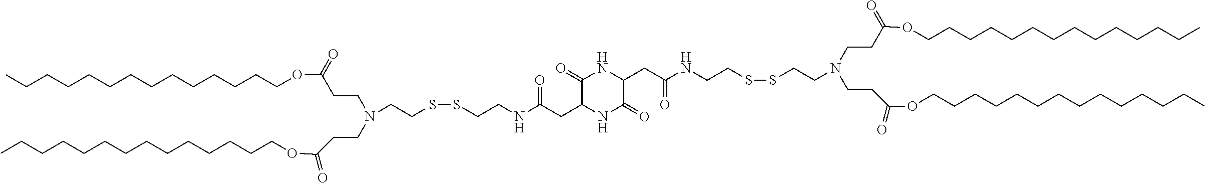 [SMILES: CCCCCCCCCCCCCCOC(=O)CCN(CCSSCCNC(=O)CC1NC(=O)C(CC(=O)NCCSSCCN(CCC(=O)OCCCCCCCCCCCCCC)CCC(=O)OCCCCCCCCCCCCCC)NC1=O)CCC(=O)OCCCCCCCCCCCCCC] | IV |
| 29 | 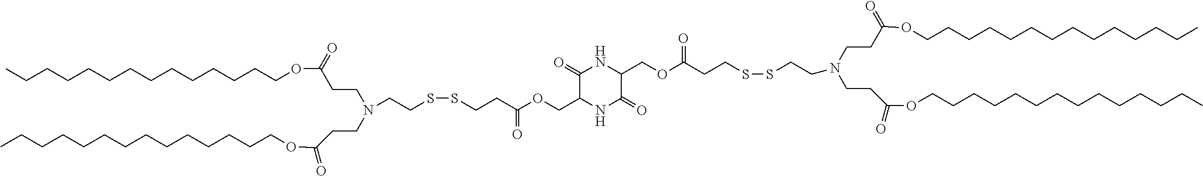 [SMILES: CCCCCCCCCCCCCCOC(=O)CCN(CCSSCCC(=O)OCC1NC(=O)C(COC(=O)CCSSCCN(CCC(=O)OCCCCCCCCCCCCCC)CCC(=O)OCCCCCCCCCCCCCC)NC1=O)CCC(=O)OCCCCCCCCCCCCCC] | IV |
| 30 | 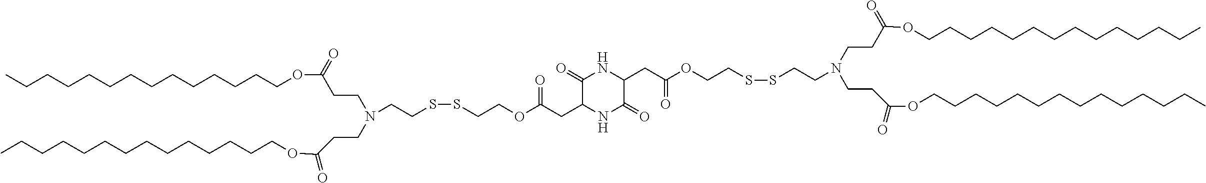 [SMILES: CCCCCCCCCCCCCCOC(=O)CCN(CCSSCCOC(=O)CC1NC(=O)C(CC(=O)OCCSSCCN(CCC(=O)OCCCCCCCCCCCCCC)CCC(=O)OCCCCCCCCCCCCCC)NC1=O)CCC(=O)OCCCCCCCCCCCCCC] | IV |
| 31 | 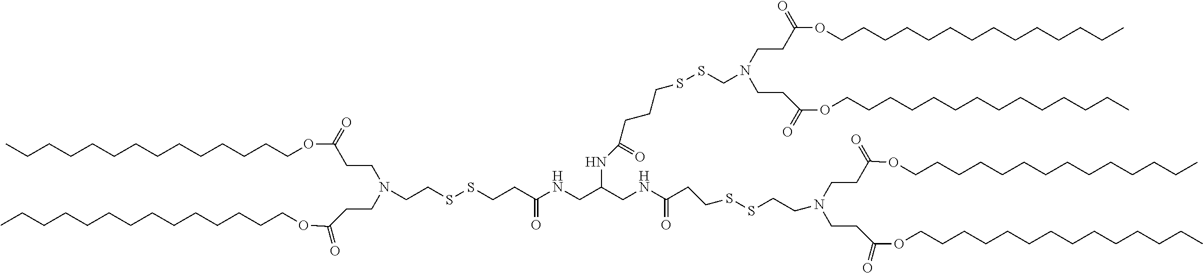 [SMILES: CCCCCCCCCCCCCCOC(=O)CCN(CCSSCCC(=O)NCC(CNC(=O)CCSSCCN(CCC(=O)OCCCCCCCCCCCCCC)CCC(=O)OCCCCCCCCCCCCCC)NC(=O)CCCSSCN(CCC(=O)OCCCCCCCCCCCCCC)CCC(=O)OCCCCCCCCCCCCCC)CCC(=O)OCCCCCCCCCCCCCC] | IV |
| 32 | 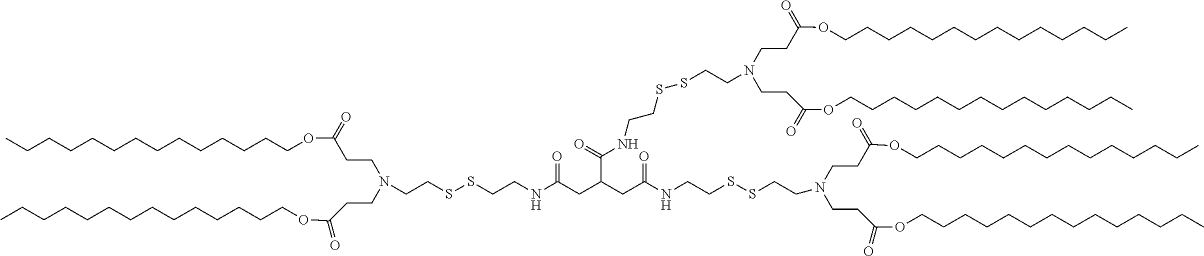 [SMILES: CCCCCCCCCCCCCCOC(=O)CCN(CCSSCCNC(=O)CC(CC(=O)NCCSSCCN(CCC(=O)OCCCCCCCCCCCCCC)CCC(=O)OCCCCCCCCCCCCCC)C(=O)NCCSSCCN(CCC(=O)OCCCCCCCCCCCCCC)CCC(=O)OCCCCCCCCCCCCCC)CCC(=O)OCCCCCCCCCCCCCC] | IV |
| 33 | 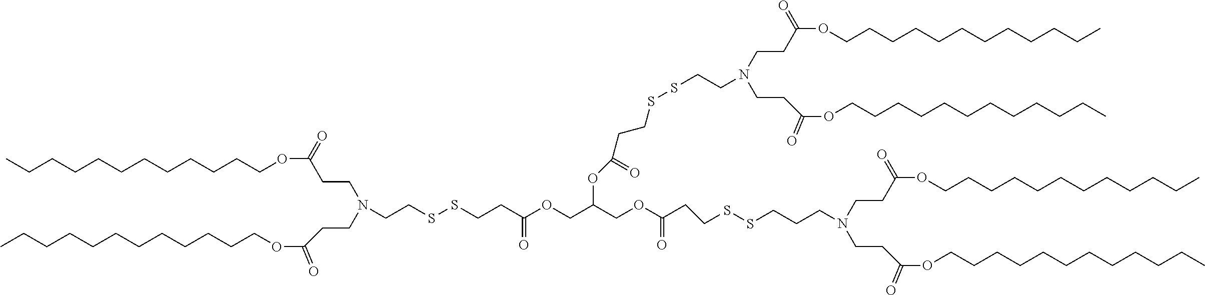 [SMILES: CCCCCCCCCCCCOC(=O)CCN(CCCSSCCC(=O)OCC(COC(=O)CCSSCCN(CCC(=O)OCCCCCCCCCCCC)CCC(=O)OCCCCCCCCCCCC)OC(=O)CCSSCCN(CCC(=O)OCCCCCCCCCCCC)CCC(=O)OCCCCCCCCCCCC)CCC(=O)OCCCCCCCCCCCC] | IV |
| 34 | 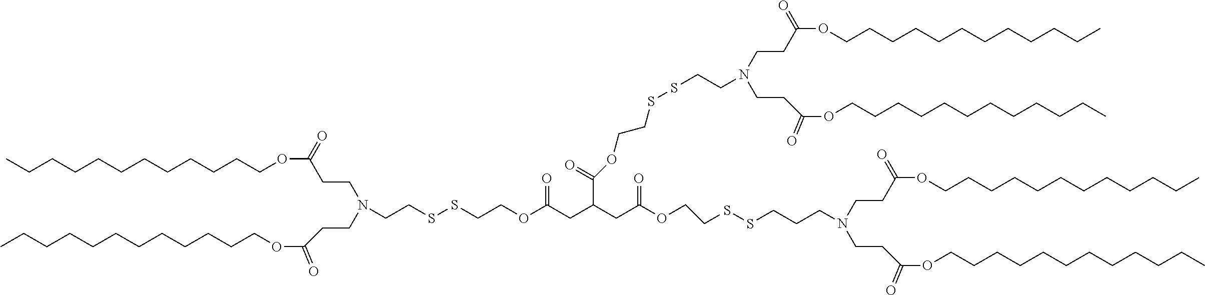 [SMILES: CCCCCCCCCCCCOC(=O)CCN(CCCSSCCOC(=O)CC(CC(=O)OCCSSCCN(CCC(=O)OCCCCCCCCCCCC)CCC(=O)OCCCCCCCCCCCC)C(=O)OCCSSCCN(CCC(=O)OCCCCCCCCCCCC)CCC(=O)OCCCCCCCCCCCC)CCC(=O)OCCCCCCCCCCCC] | IV |
| 35 | 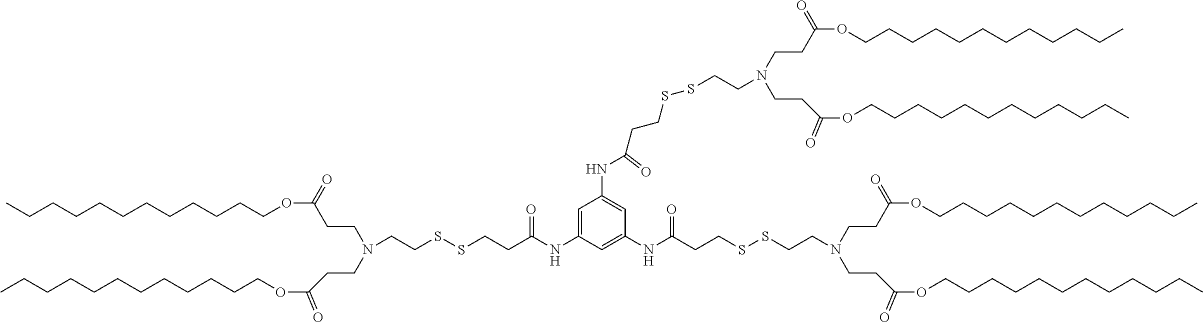 [SMILES: CCCCCCCCCCCCOC(=O)CCN(CCSSCCC(=O)Nc1cc(NC(=O)CCSSCCN(CCC(=O)OCCCCCCCCCCCC)CCC(=O)OCCCCCCCCCCCC)cc(NC(=O)CCSSCCN(CCC(=O)OCCCCCCCCCCCC)CCC(=O)OCCCCCCCCCCCC)c1)CCC(=O)OCCCCCCCCCCCC] | IV |
| 36 | 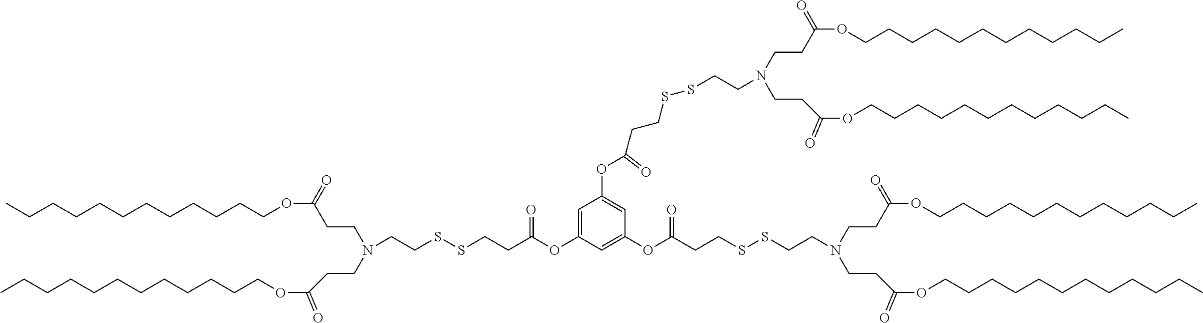 [SMILES: CCCCCCCCCCCCOC(=O)CCN(CCSSCCC(=O)Oc1cc(OC(=O)CCSSCCN(CCC(=O)OCCCCCCCCCCCC)CCC(=O)OCCCCCCCCCCCC)cc(OC(=O)CCSSCCN(CCC(=O)OCCCCCCCCCCCC)CCC(=O)OCCCCCCCCCCCC)c1)CCC(=O)OCCCCCCCCCCCC] | IV |
| 37 | 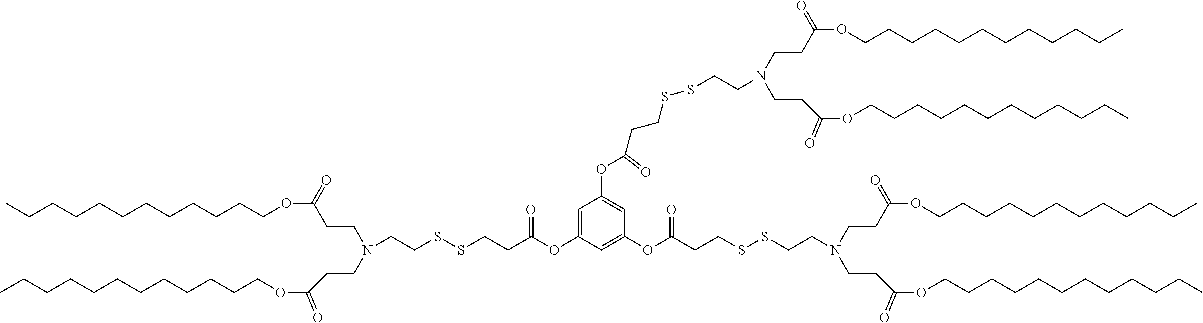 [SMILES: CCCCCCCCCCCCOC(=O)CCN(CCSSCCC(=O)Oc1cc(OC(=O)CCSSCCN(CCC(=O)OCCCCCCCCCCCC)CCC(=O)OCCCCCCCCCCCC)cc(OC(=O)CCSSCCN(CCC(=O)OCCCCCCCCCCCC)CCC(=O)OCCCCCCCCCCCC)c1)CCC(=O)OCCCCCCCCCCCC] | IV |
| 38 | 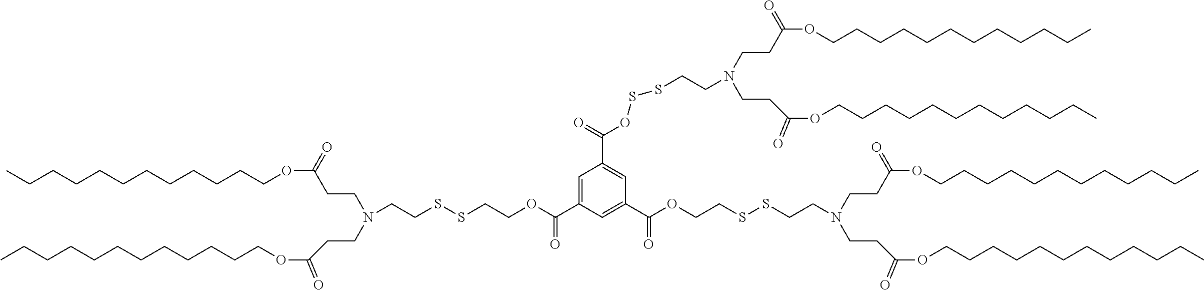 [SMILES: CCCCCCCCCCCCOC(=O)CCN(CCSSCCOC(=O)c1cc(C(=O)OCCSSCCN(CCC(=O)OCCCCCCCCCCCC)CCC(=O)OCCCCCCCCCCCC)cc(C(=O)OSSCCN(CCC(=O)OCCCCCCCCCCCC)CCC(=O)OCCCCCCCCCCCC)c1)CCC(=O)OCCCCCCCCCCCC] | IV |
| 39 | 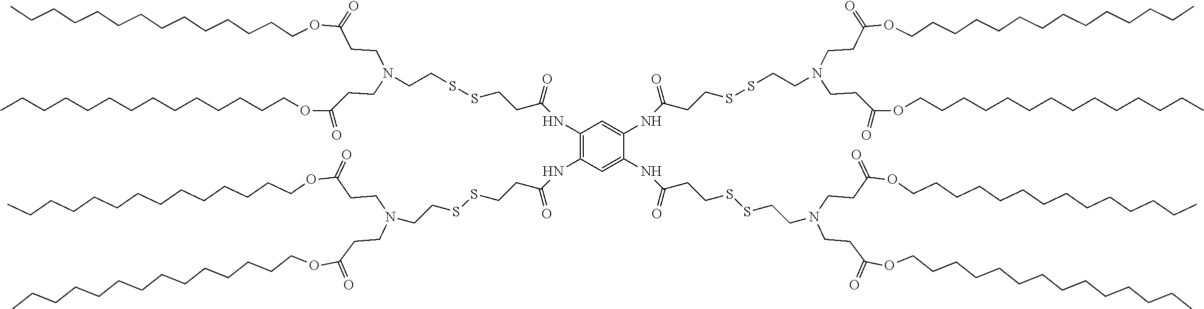 [SMILES: CCCCCCCCCCCCCCOC(=O)CCN(CCSSCCC(=O)Nc1cc(NC(=O)CCSSCCN(CCC(=O)OCCCCCCCCCCCCCC)CCC(=O)OCCCCCCCCCCCCCC)c(NC(=O)CCSSCCN(CCC(=O)OCCCCCCCCCCCCCC)CCC(=O)OCCCCCCCCCCCCCC)cc1NC(=O)CCSSCCN(CCC(=O)OCCCCCCCCCCCCCC)CCC(=O)OCCCCCCCCCCCCCC)CCC(=O)OCCCCCCCCCCCCCC] | IV |
| 40 | 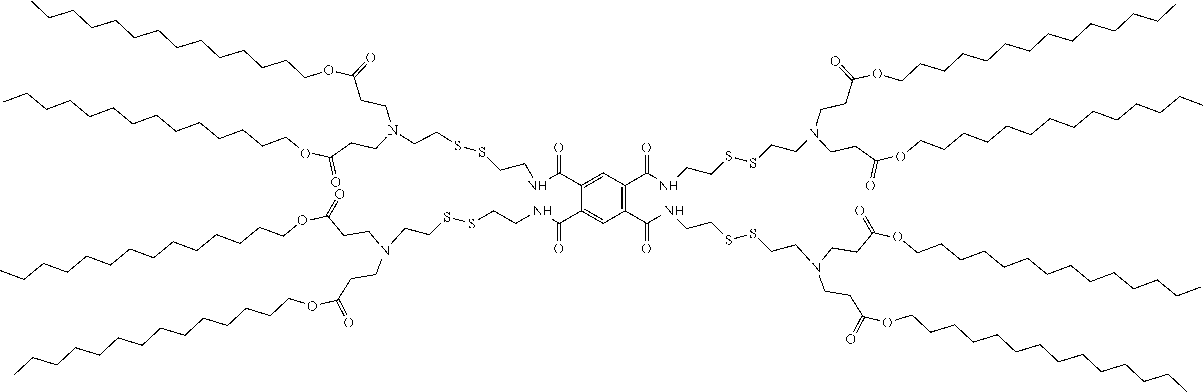 [SMILES: CCCCCCCCCCCCCCOC(=O)CCN(CCSSCCNC(=O)c1cc(C(=O)NCCSSCCN(CCC(=O)OCCCCCCCCCCCCCC)CCC(=O)OCCCCCCCCCCCCCC)c(C(=O)NCCSSCCN(CCC(=O)OCCCCCCCCCCCCCC)CCC(=O)OCCCCCCCCCCCCCC)cc1C(=O)NCCSSCCN(CCC(=O)OCCCCCCCCCCCCCC)CCC(=O)OCCCCCCCCCCCCCC)CCC(=O)OCCCCCCCCCCCCCC] | IV |
| 41 | 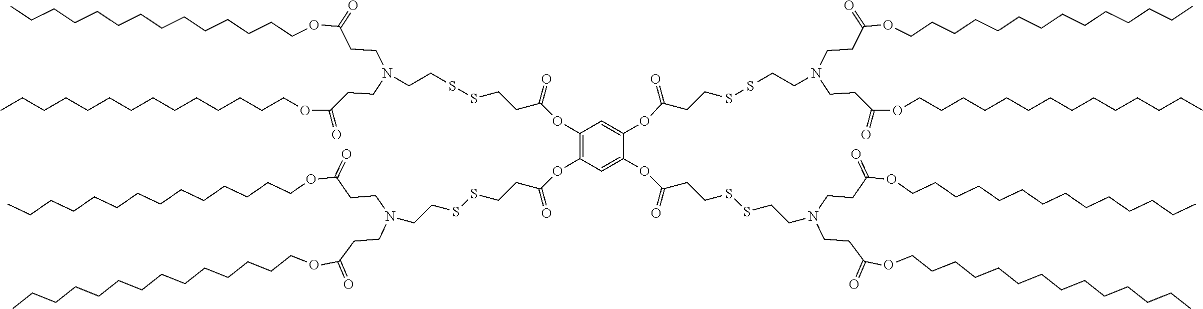 [SMILES: CCCCCCCCCCCCCCOC(=O)CCN(CCSSCCC(=O)Oc1cc(OC(=O)CCSSCCN(CCC(=O)OCCCCCCCCCCCCCC)CCC(=O)OCCCCCCCCCCCCCC)c(OC(=O)CCSSCCN(CCC(=O)OCCCCCCCCCCCCCC)CCC(=O)OCCCCCCCCCCCCCC)cc1OC(=O)CCSSCCN(CCC(=O)OCCCCCCCCCCCCCC)CCC(=O)OCCCCCCCCCCCCCC)CCC(=O)OCCCCCCCCCCCCCC] | IV |
| 42 | 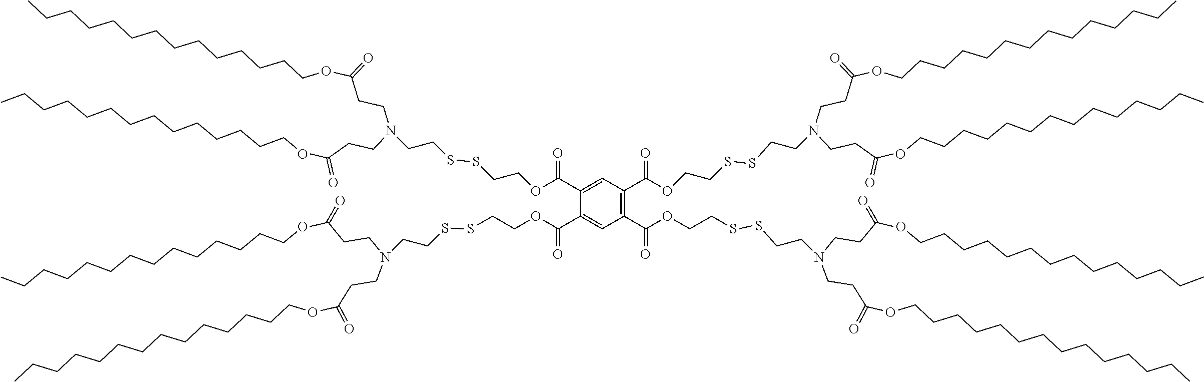 [SMILES: CCCCCCCCCCCCCCOC(=O)CCN(CCSSCCOC(=O)c1cc(C(=O)OCCSSCCN(CCC(=O)OCCCCCCCCCCCCCC)CCC(=O)OCCCCCCCCCCCCCC)c(C(=O)OCCSSCCN(CCC(=O)OCCCCCCCCCCCCCC)CCC(=O)OCCCCCCCCCCCCCC)cc1C(=O)OCCSSCCN(CCC(=O)OCCCCCCCCCCCCCC)CCC(=O)OCCCCCCCCCCCCCC)CCC(=O)OCCCCCCCCCCCCCC] | IV |

### Hydrophobic Tail Moiety (HTM)

The single-disulfide CDM compounds and multi-disulfide CDM compounds have one or more hydrophobic tail moieties (HTMs). In some embodiments, HTM is a C₅₋₄₀ linear or branched alkyl, C₅₋₄₀ linear or branched alkenyl, or C₅₋₄₀ linear or branched alkynyl.

In some embodiments, HTM is a C₅₋₄₀ linear or branched alkyl. In some embodiments, HTM is a C₅₋₃₀ linear alkyl. In some embodiments, HTM is a C₅₋₂₀ linear alkyl. In some embodiments, HTM is a C₈₋₂₀ linear alkyl. In some embodiments, HTM is a C₁₀₋₂₀ linear alkyl. In some embodiments, HTM is a C₁₂₋₂₀ linear alkyl. In some embodiments, HTM is a C₁₄₋₂₀ linear alkyl. In some embodiments, HTM is a C₁₆₋₂₀ linear alkyl. In some embodiments, HTM is a C₁₆₋₁₈ linear alkyl.

In some embodiments, HTM is a C₁₀₋₃₀ branched alkyl. In some embodiments, the branched alkyl has one C₅₋₁₅ alkyl branch on a C₅₋₁₅ alkyl main chain. In some embodiments, HTM is a C₁₅₋₄₀ branched alkyl. In some embodiments, the branched alkyl has two C₅₋₁₅ alkyl branches on a C₅₋₁₅ alkyl main chain.

In some embodiments, the linear or branched alkyl, alkenyl, or alkynyl has one or more intervening groups selected from ester —(C═O)—O—, amide —(C═O)—NR—, or carboxylate —O—(C═O)—O—. In some embodiments, one or more intervening groups is selected from ester —(C═O)—O— or amide —(C═O)—NR—. In some embodiments, the one or more intervening group is inserted in the alkyl such that the acyl group is connect to nitrogen in Formula I or Formula II through an ethylene —CH2—CH2—. In some embodiments, the one or more intervening group is one or more ester group.

In some embodiments, HTM has the structure of:

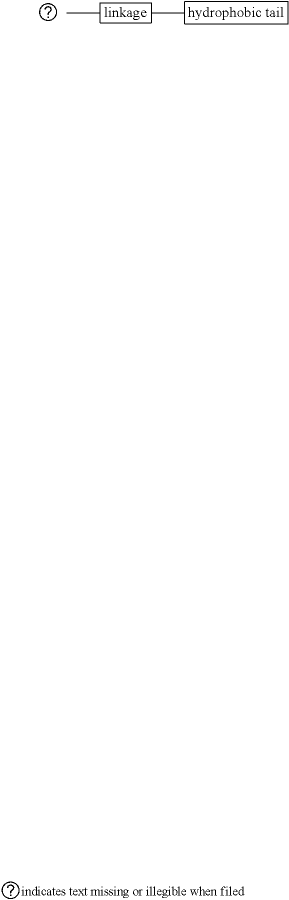

wherein the linkage is selected from

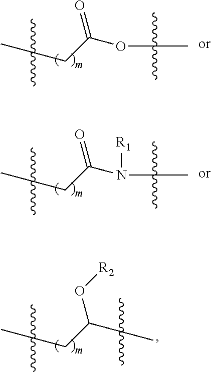

m is an integer of 1-6: R₁ is H, CH₃, or CH₂CH₃; and R₂ is H, CH₃, CH₂CH₃, or (C═O)CH₃ wherein the hydrophobic tail is selected from the group consisting of:

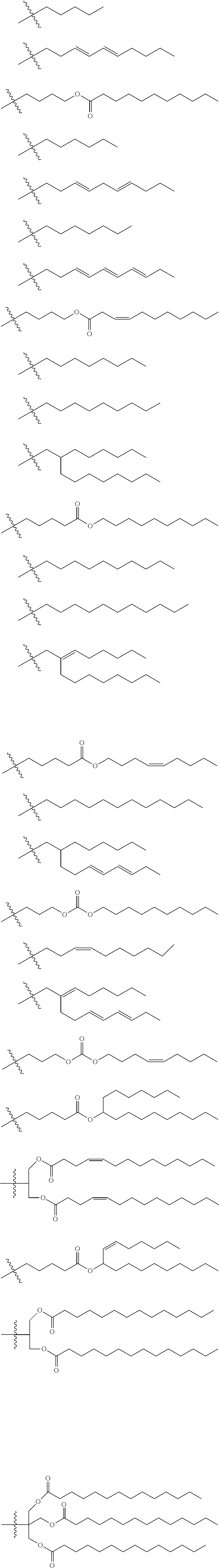

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Methods known to one of ordinary skill in the art are identified through various reference books and databases. Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In some instances, specific and analogous reactants are identified through the indices of known chemicals prepared by the Chemical Abstract Service of the American Chemical Society, which are available in most public and university libraries, as well as through on-line databases (the American Chemical Society, Washington, D.C., is contacted for more details). Chemicals that are known but not commercially available in catalogs are prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

Below are some non-limiting examples of reaction schemes to prepare the compounds disclosed herein.

### Type I CDM Compounds

Type I CDM Compounds can be prepared by a two-step process as shown below:

Type I CDMs can be prepared through a two-step process:

SM-1+SM-2→IM-1 (LG-S—S-L-NR_{c}R_{d})
Step 1 can be done through Michael addition or ring opening reaction.
One example,

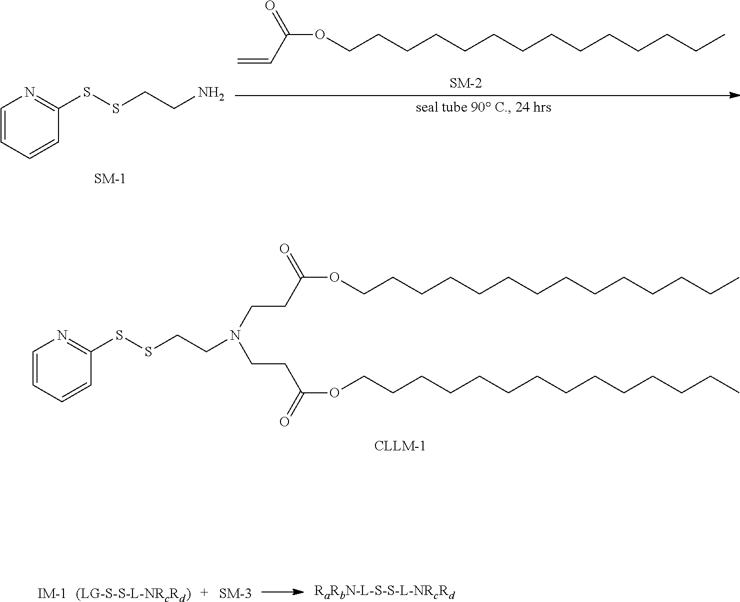

Step 2 can be done through a disulfide exchange reaction.
One example,

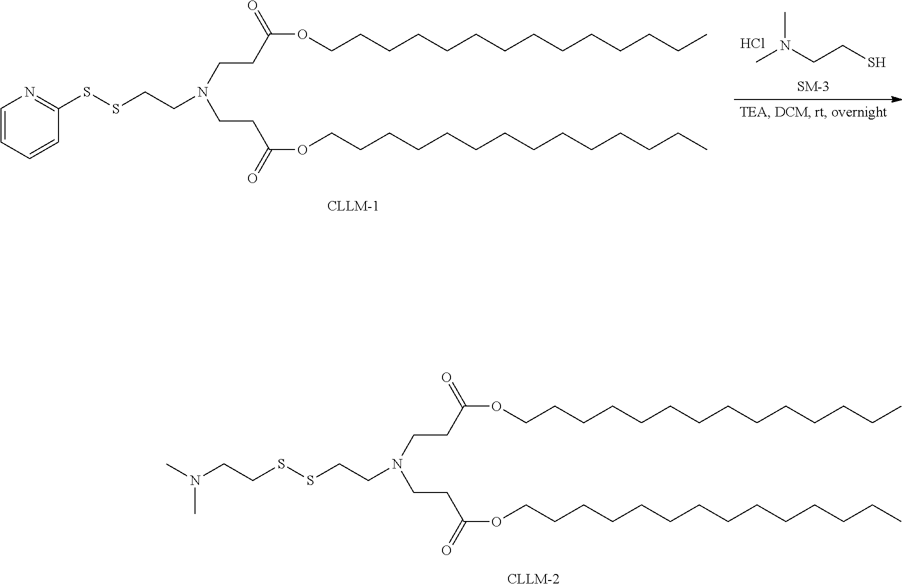

Non-limiting examples of SM1 include, but are not limited to:

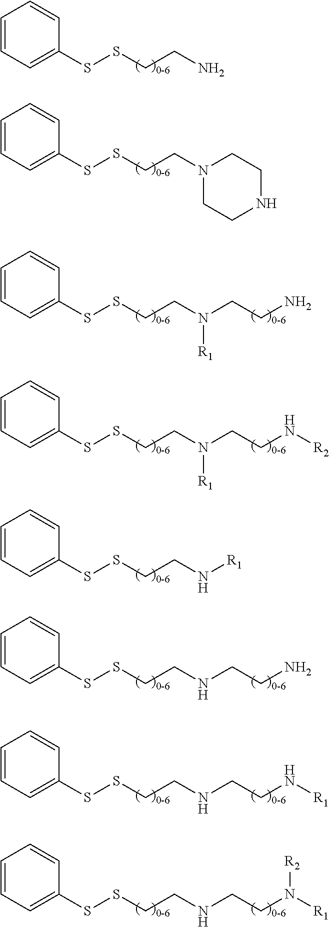

R₁ and R₂, independently, are:
a C₁-C₁₀ monovalent aliphatic radical, such as

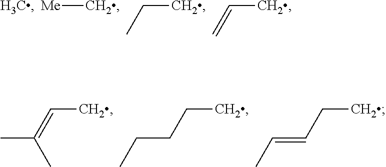

a C₁-C₁₀ monovalent heteroaliphatic radical, such as

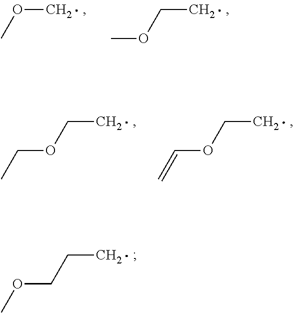

a monovalent aryl radical, such as

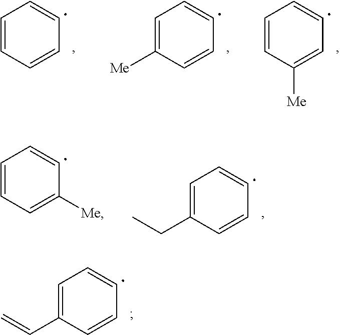

or a monovalent heteroaryl radical, such as

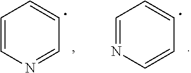

Non-limiting examples of SM2 include, but are not limited to:

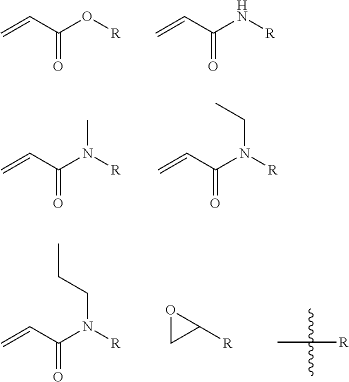

group could be one of hydrophobic tails

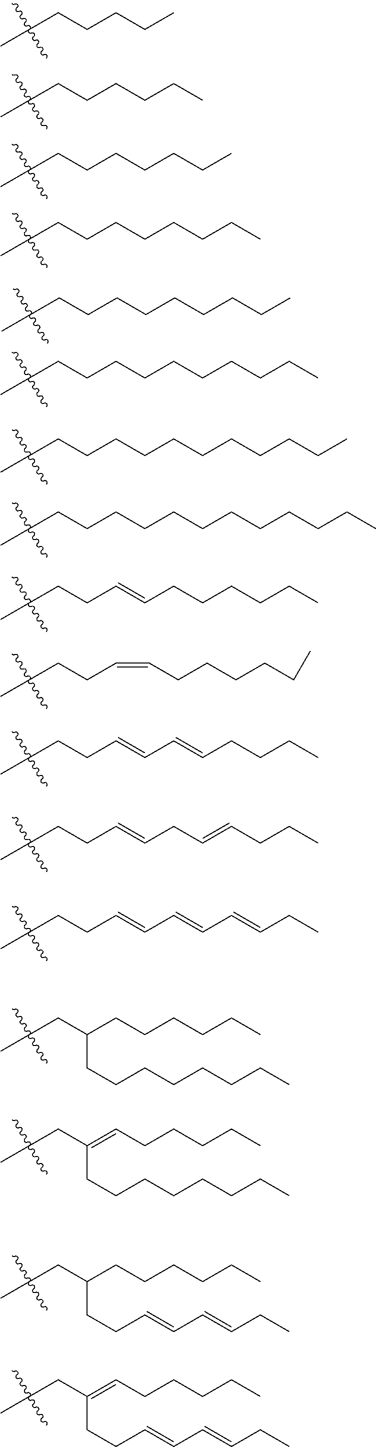

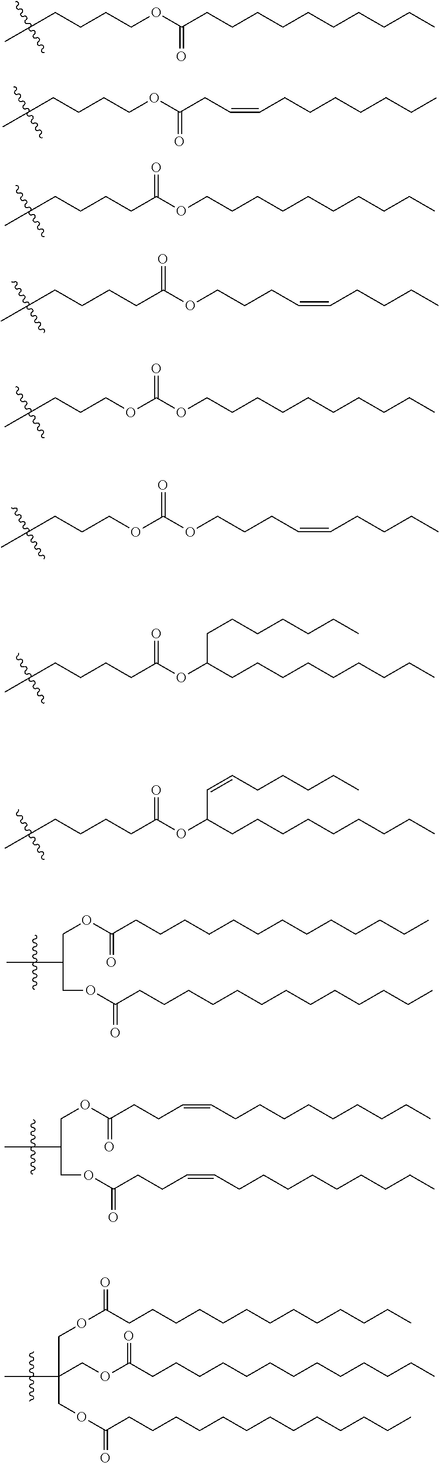

Non-limiting examples of SM-3 inlcude, but are not limited to:

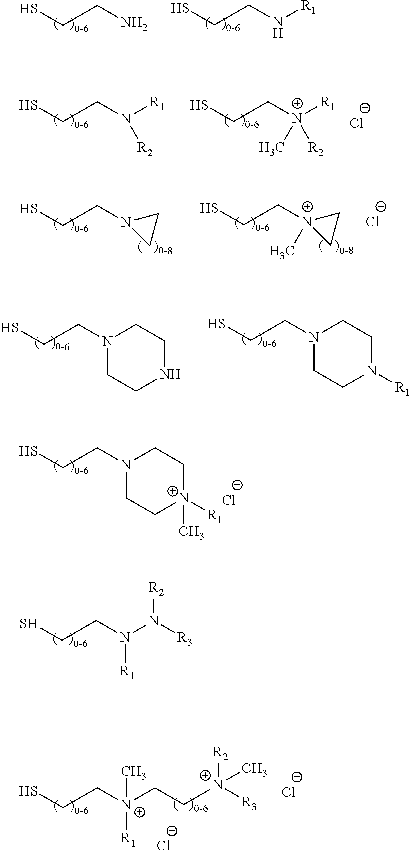

R₁, R₂, and R3, independently, are:
a C₁-C₁₀ monovalent aliphatic radical, such as

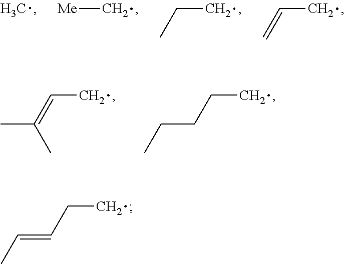

a C₁-C₁₀ monovalent heteroaliphatic radical, such as

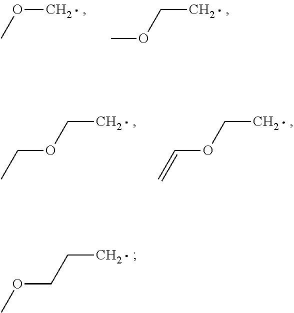

a monovalent aryl radical, such as

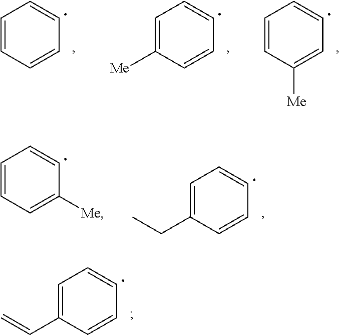

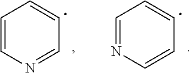

or a monovalent heteroaryl radical, such as

### Type II CDM Compound

Type II CDM compounds can be prepared by the same two-step process as Type I CDM compounds above, but with the SM-3 have one or more HTMs.

Alternatively, Type II CDM compounds can be prepared without the disulfide exchange reaction. Below is a non-limiting example:

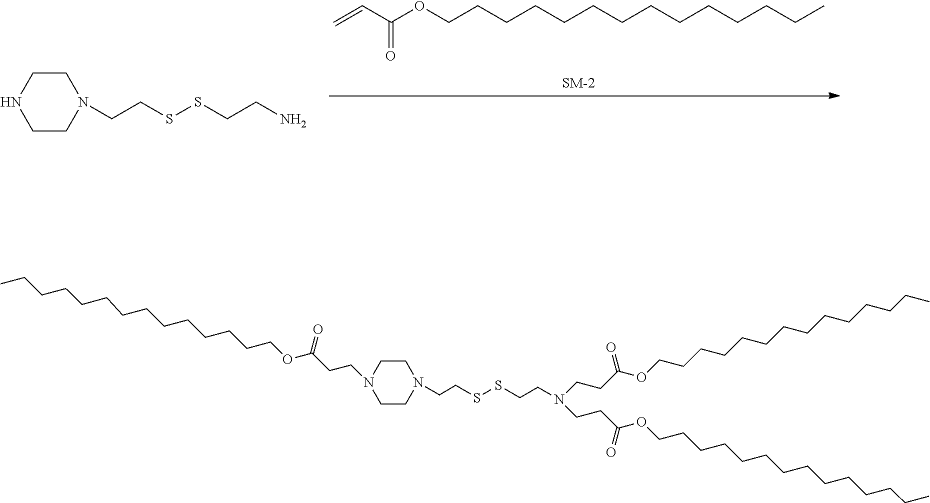 [SMILES: C=CC(=O)OCCCCCCCCCCCCCC ; NCCSSCCN1CCNCC1 ; CCCCCCCCCCCCCCOC(=O)CCN(CCSSCCN1CCN(CCC(=O)OCCCCCCCCCCCCCC)CC1)CCC(=O)OCCCCCCCCCCCCCC]

### Type IV CDM Compound

Type IV CDM compounds can be made through a three-step process. The first step is similar to the preparation of Type I compound. Step 2 is the disulfide exchange reaction similar to the preparation of Type I compound to form intermediate 2 (IM-2), but with SM-3 selected from

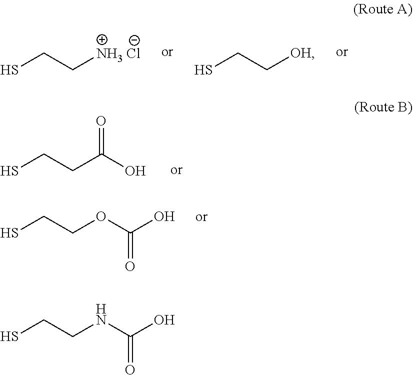

The third step is the coupler of IM-2 to the multi-valent core (MVC) to obtain the Type IV compounds. Below is an example of the overall reaction scheme:

### Step 1

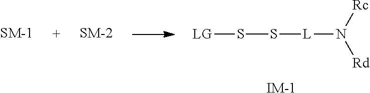

### Step 2

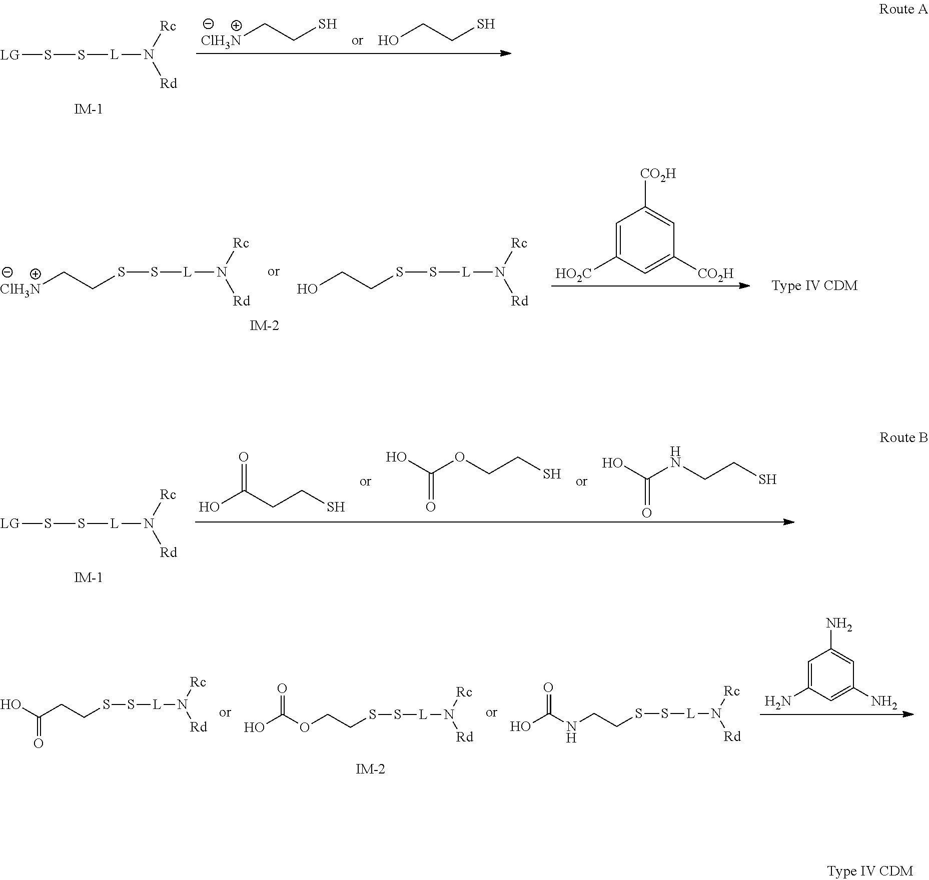

Below is an example of the reaction scheme through Route A:

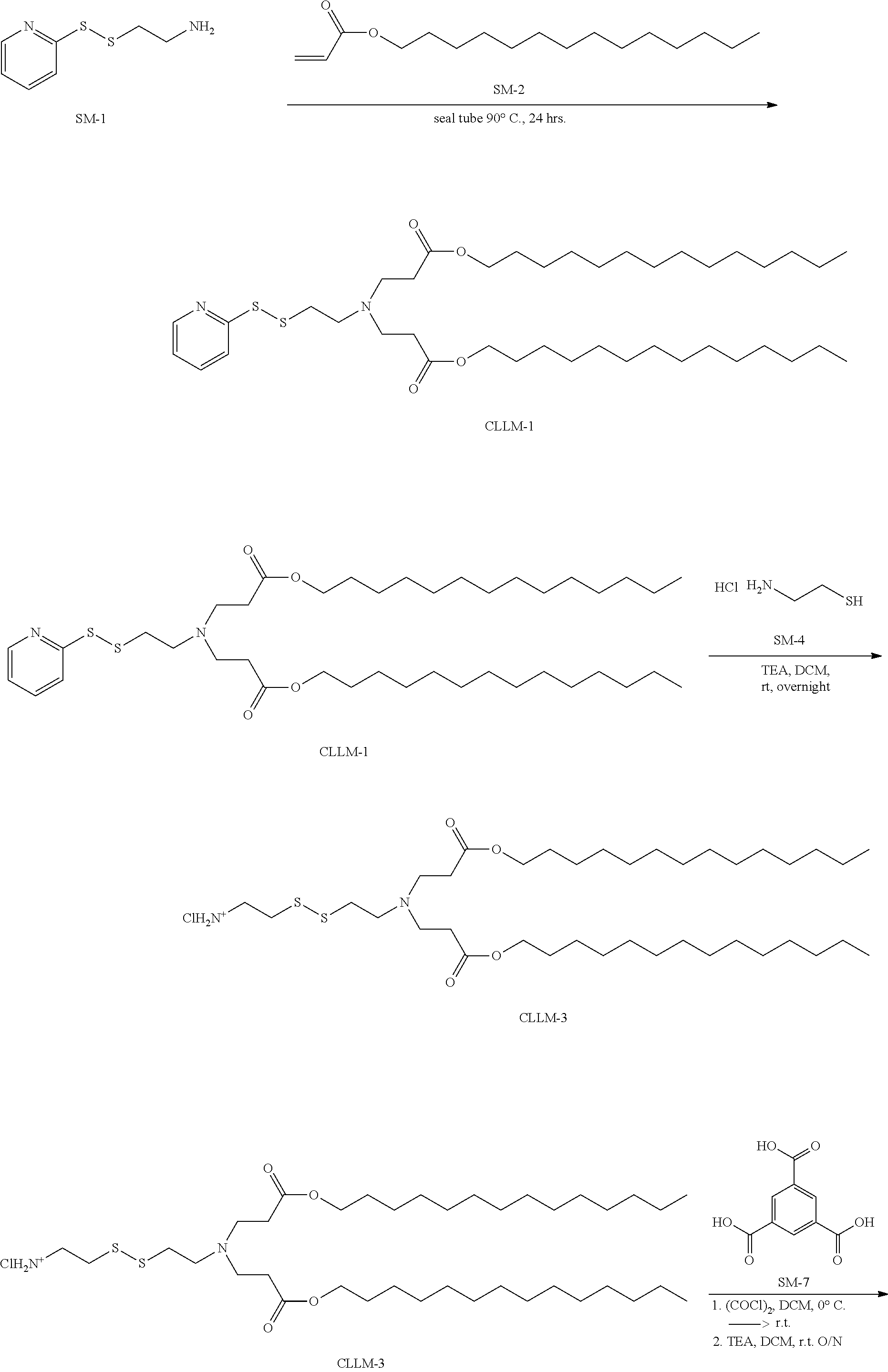

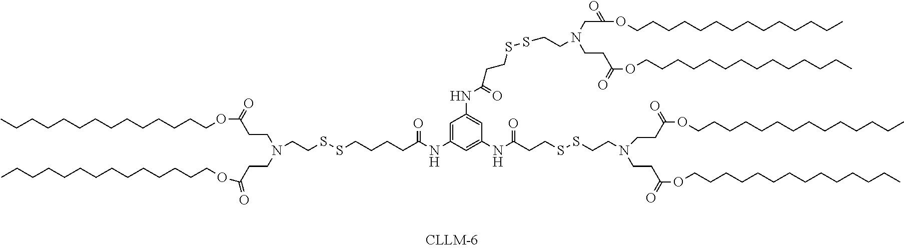 [SMILES: CCCCCCCCCCCCCCOC(=O)CCN(CCSSCCCCC(=O)Nc1cc(NC(=O)CCSSCCN(CCC(=O)OCCCCCCCCCCCCCC)CCC(=O)OCCCCCCCCCCCCCC)cc(NC(=O)CCSSCCN(CCC(=O)OCCCCCCCCCCCCCC)CC(=O)OCCCCCCCCCCCCCC)c1)CCC(=O)OCCCCCCCCCCCCCC]

### Further Forms of Disclosed CDM Compounds

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. In some embodiments, examples of isotopes that are incorporated into compounds of the disclosure include isotopes of hydrogen, carbon, nitrogen, oxygen, phosphorous, sulfur, fluorine and chloride, such as ²H, ³H, ¹³C , ¹⁴C , ¹⁵N , ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e. , ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the disclosure, or by separately reacting a purified compound in its free form with a suitable acid or base, and isolating the salt thus formed.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. In some embodiments, solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In some embodiments, the compounds provided herein exist in unsolvated as well as solvated forms. In general, the solvated forms are considered equivalent to the unsolvated forms for the purposes of the compounds and methods provided herein.

### Drug Delivery Using Disclosed CDM Compounds

The CDM compounds disclosed herein can package and deliver nucleic acid and other large molecule or small molecule drugs intracellularly to the targets. The present disclosure recognizes that disulfide bonds are subjective to cleavage in a reductive environment. The concentration of glutathione mM) in cytoplasm is nearly 5,000-fold higher than in extracellular environments (˜2 μM), which makes the cytoplasm very reductive (ANNU. REV. BIOCHEM. 1983, 52, 711). The cleavage of an artificial disulfide molecule injected to cytoplasm reached completion within minutes (J. AM. CHEM. SOC. 2008, 130, 2398).

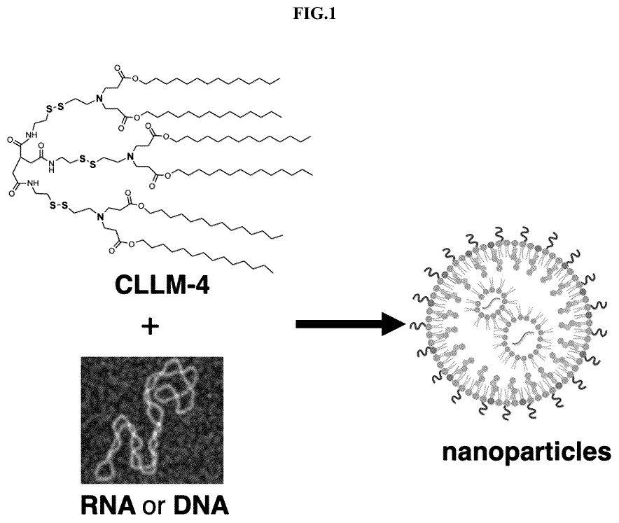

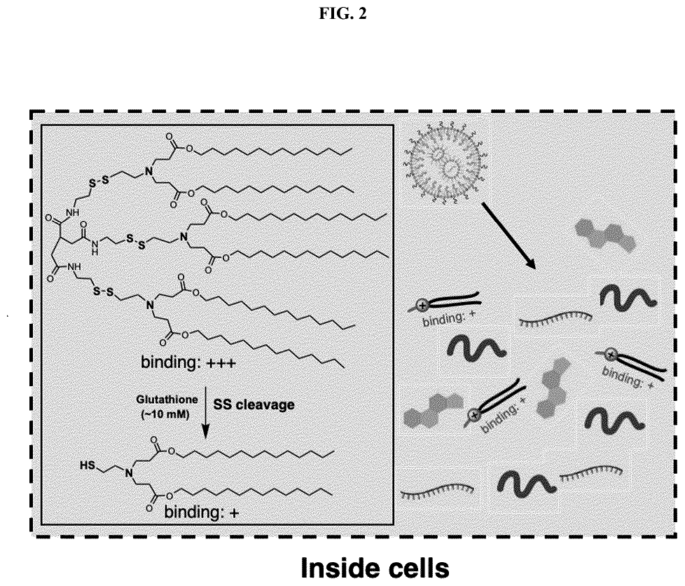

As shown in FIGS. 1 and 2, these disulfide bonds-containing cleavable lipid-like materials have multiple ionizable nucleic acid-binding amino groups. The cleavable lipid-like materials form delivery nanoparticles with effective packing of nucleic acid molecules (RNAs or DNAs). When reaching the cytoplasm inside the cell that have a highly reductive environment, the disulfide bonds are broken by the glutathione and the lipid-like materials are decomposed within the nucleic acid binding site. It enables the release of its cargos.

### Lipid Nanoparticle Formulations

In some embodiments, nanoparticle formulations comprising CDM compounds disclosed herein, phospholipids, polyethylene glycol-lipids, and cholesterol is provided. In some embodiments, the nanoparticle formulations disclosed herein are capable of delivering nucleic acid molecules from 1 to 500 k nucleotides, such as cyclic dinucleotides, messenger RNAs, and plasmid DNAs through the combination of charge interaction and hydrophobic interaction.

In some embodiments, the nanoparticle formulations disclosed herein are capable of delivering peptides and proteins through the combination of charge interaction and hydrophobic interaction.

In some embodiments, the nanoparticle formulations disclosed herein are capable of delivering other large molecules or small molecules through charge interaction, hydrophobic interaction, or its combination.

Without wishing to be bound by any particular theory, the present disclosure recognizes that delivery for nucleic acid molecules and other types of cargos faces two primary challenges (besides other barriers and challenges) in some application: packing and releasing its cargos effectively after crossing the cell membrane. Furthermore, the present disclosure recognizes that packing and releasing the nucleic acid molecules are two different, and sometimes contradictory, processes.

The CDM compounds disclosed herein are designed and synthesized with cleavable disulfide bonds within its nucleic acid binding site. These materials have strong packing of nucleic acid molecules. When reaching the cytoplasm inside the cell that have a reductive environment, the materials are decomposed from the nucleic acid binding site to release its cargos. These cleavable lipid-like materials solve the dilemma of packing and releasing drug payloads, such as nucleic acid therapeutics.

### Pharmaceutical Compositions

In certain embodiments, nanoparticles based on the CDM compounds as described herein is administered as a pure chemical. In other embodiments, the nanoparticles based on the CDM compounds described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)), the disclosure of which is hereby incorporated herein by reference in its entirety.

Accordingly, provided herein is a pharmaceutical composition comprising nanoparticles based on the CDM compounds described herein, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and nanoparticles based on the Type I CDM compounds, or a pharmaceutically acceptable salt thereof.

One embodiment provides a pharmaceutical composition comprising a pharmaceutically acceptable carrier and nanoparticles based on the Type II CDM compounds, or a pharmaceutically acceptable salt thereof.

One embodiment provides a pharmaceutical composition comprising a pharmaceutically acceptable carrier and nanoparticles based on the Type III CDM compounds, or a pharmaceutically acceptable salt thereof.

One embodiment provides a pharmaceutical composition comprising a pharmaceutically acceptable carrier and nanoparticles based on the Type IV CDM compounds, or a pharmaceutically acceptable salt thereof.

These pharmaceutical compositions include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions are formulated as a unit dose, and/or are formulated for oral or subcutaneous administration.

In some instances, exemplary pharmaceutical compositions are used in the form of a pharmaceutical preparation, for example, in solid, semisolid or liquid form, which includes one or more of the disclosed nanoparticle formulations, as an active ingredient, in admixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The nanoparticle formulation disclosed herein is included in the pharmaceutical composition in an amount sufficient to produce the desired effect upon the process or condition of the disease.

For preparing solid compositions such as tablets in some instances, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions also comprise buffering agents in some embodiments. Solid compositions of a similar type are also employed as fillers in soft and hard-filled gelatin capsules using such excipients as lactose or milk sugars, as well as high molecular weight polyethylene glycols and the like.

In some instances, a tablet is made by compression or molding, optionally with one or more accessory ingredients. Compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets are made by molding in a suitable machine a mixture of the subject composition moistened with an inert liquid diluent. Tablets, and other solid dosage forms, such as dragees, capsules, pills and granules, are optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms contain optionally inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, optionally contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for transdermal administration of a subject composition include powders, sprays, ointments, pastes, creams, lotions, gels, solutions, patches and inhalants. The active component is optionally mixed under sterile conditions with a pharmaceutically acceptable carrier, and with any preservatives, buffers, or propellants which are required in some embodiments.

In some embodiments, the ointments, pastes, creams and gels contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, powders and sprays contain, in addition to a subject composition, excipients such as lactose, talc, silicic acid, aluminum hydroxide, calcium silicates and polyamide powder, or mixtures of these substances. Sprays additionally contain customary propellants, such as chlorofluorohydrocarbons and volatile unsubstituted hydrocarbons, such as butane and propane.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which optionally contain antioxidants, buffers, bacteriostats, solutes which render the formulation isotonic with the blood of the intended recipient or suspending or thickening agents.

Examples of suitable aqueous and non-aqueous carriers employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. In some embodiments, proper fluidity is maintained, for example, by the use of coating materials, such as lecithin, by the maintenance of the required particle size in the case of dispersions, and by the use of surfactants. Therapeutic Uses

Without wishing to be bound by any particular theory, it is contemplated that the CDM compounds disclosed herein provides a delivery drug platform for many nucleic acid drugs and other large molecule or small molecule drugs, and provided desirable delivery of therapeutics (e.g. larger payload, less degradation, and/or less side effect) for cancers, genetic diseases, infectious diseases, and other diseases and conditions.

In some embodiments, the dose of the composition comprising at least one compound as described herein differ, depending upon the patient's (e.g., human) condition, that is, stage of the disease, general health status, age, and other factors that a person skilled in the medical art will use to determine dose.

In some instances, pharmaceutical compositions are administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

### Non-Limiting Embodiments

The embodiments described herein are for illustrative purposes only and various modifications or changes suggested to persons skilled in the art are to be included within the spirit and purview of this application and scope of the appended claims. These embodiments are provided for illustrative purposes only and not to limit the scope of the claims provided herein.
1. A compound of Formula I, a stereoisomer or pharmaceutically acceptable salt thereof:

RₐR_{b}N-L₁-S—S-L₂-NR_{c}R_{d} (Formula I)

wherein L₁ and L₂ are each independently C₁₋₄ alkylenyl;

wherein at least one of Rₐ, R_{b}, R_{c} and R_{d} is independently a hydrophobic tail moiety (HTM) or combined with nitrogen on the same side of Formula I to form a 3-member to 8-member heterocyclic ring substituted with an HTM, and the wherein the remainder of Rₐ, R_{b}, R_{c} and R_{d} is independently a hydrogen, a C₁₋₄ alkyl, or combine with nitrogen on the same side of Formula I to form a 3-member to 8-member heterocyclic ring optionally substituted with a C₁₋₄ alkyl,

wherein HTM is a C₅₋₄₀ linear or branched alkyl, C₅₋₄₀ linear or branched alkenyl, or C₅₋₄₀ linear or branched alkynyl; or wherein HTM has a structure of Formula II:

-L-NR′R″ (Formula II)
wherein L is C₁₋₄ alkylenyl and at least one of R′ and R″ is a C₅₋₄₅ linear or branched alkyl, C₅₋₄₅ linear or branched alkenyl, or C₅₋₄₅ linear or branched alkynyl.
2. The compound of embodiment 1, wherein L₁ and L₂ are each independently C₁₋₃ alkylenyl.
3. The compound of embodiment 2, wherein L1 is ethylene.
4. The compound of embodiment 2-3, wherein L2 is ethylene.
5. The compound of embodiment 1-4, wherein Rₐ and R_{b} is independently a hydrogen, a C₁₋₄ alkyl, or combine with nitrogen on the same side of Formula I to form a 3-member to 8-member heterocyclic ring optionally substituted with a C₁₋₄ alkyl.
6. The compound of embodiment 5, wherein Rₐ and R_{b} are hydrogen.
7 The compound of embodiment 5, wherein the Rₐ is hydrogen and wherein R_{b} is a C₁₋₄ alkyl.
8. The compound of embodiment 7-8, wherein R_{b} is a butyl.
9 The compound of embodiment 5, wherein Rₐ and R_{b} are each independently a C₁₋₄ alkyl.
10. The compound of embodiment 5, wherein Rₐ and R_{b} are methyl.
11. The compound of embodiment 5, wherein Rₐ and R_{b} combine with nitrogen on the same side of Formula I to form a 3-member to 8-member heterocyclic ring.
12. The compound of embodiment 11, wherein the heterocyclic ring is substituted with a C₁₋₄ alkyl.
13. The compound of embodiment 11-12, wherein the heterocyclic ring is a 5-member to 7-member ring.
14. The compound of embodiment 11-12, wherein the heterocyclic ring is a 6-member ring.
15. The compound of embodiment 11-12, wherein the heterocyclic ring is a diazinane ring.
16. The compound of embodiment 11-12, wherein the diazinane ring is a 1,4-diazinane ring.
17. The compound of embodiment 16, wherein the 1,4-diazinane ring is substituted with a C₁₋₄ alkyl at the 4 position.
18. The compound of embodiment 5-17, wherein R_{c} is an HTM and Rd is a hydrogen or a C1-4 alkyl.
19. The compound of embodiment 5-17, wherein R_{c} and R_{d} are each independently an HTM.
20. The compound of embodiment 5-17, wherein R_{c} and R_{d} combined with nitrogen on the same side of Formula I to form a 3-member to 8-member heterocyclic ring substituted with an HTM.
21. The compound of embodiment 20, wherein the heterocyclic ring is a 5-member to 7-member ring.
22. The compound of embodiment 20, wherein the heterocyclic ring is a 6-member ring.
23. The compound of embodiment 20, wherein the heterocyclic ring is a diazinane ring.
24. The compound of embodiment 20, wherein the diazinane ring is a 1,4-diazinane ring.
25. The compound of embodiment 24, wherein the 1,4-diazinane ring is substituted with the HTM at the 4 position.
26. The compound of embodiment 1-4, wherein at least one of Rₐ and R_{b} is an HTM or Rₐ and R_{b} combine with nitrogen on the same side of Formula one to form a 3-member to 8-member heterocyclic ring substituted with an HTM, and wherein at least one of R_{c} and R_{d} is an HTM or R_{c} and R_{d} combine with nitrogen on the same side of Formula one to form a 3-member to 8-member heterocyclic ring substituted with an HTM.
27. The compound of embodiment 26, wherein at least one of Rₐ and R_{b} is an HTM, and wherein at least one of R_{c} and R_{d} is an HTM.
28. The compound of embodiment 27, wherein Rₐ is an HTM and R_{b} is hydrogen or a C₁₋₄ alkyl
29. The compound of embodiment 28, wherein R_{b} is hydrogen.
30. The compound of clam 28, wherein R_{b} is a C₁₋₄ alkyl.
31. The compound of embodiment 28-30, wherein R_{c} is an HTM and R_{d} is hydrogen or a C₁₋₄ alkyl
32. The compound of embodiment 31, wherein R_{d} is hydrogen.
33. The compound of clam 31, wherein R_{d} is a C₁₋₄ alkyl.
34. The compound of embodiment 30 or 33, wherein the C₁₋₄ alkyl is methyl.
35. The compound of embodiment 30 or 33, wherein the C₁₋₄ alkyl is butyl.
36. The compound of embodiment 28-30, wherein R_{c} and R_{d} are each independently an HTM.
37. The compound of embodiment 27, wherein Rₐ, R_{b}, R_{c} and R_{d} are each independently an HTM.
38. The compound of embodiment 26, wherein at least one of Rₐ and R_{b} is an HTM, and R_{c} and R_{d} combine with nitrogen on the same side of Formula one to form a 3-member to 8-member heterocyclic ring substituted with an HTM.
39. The compound of embodiment 38, wherein Rₐ is an HTM and R_{b} is hydrogen or a C₁₋₄ alkyl
40. The compound of embodiment 39, wherein R_{b} is hydrogen.
41. The compound of clam 39, wherein R_{b} is a C₁₋₄ alkyl.
42. The compound of embodiment 41, wherein the C₁₋₄ alkyl is methyl.
43. The compound of embodiment 41, wherein the C₁₋₄ alkyl is butyl.
44. The compound of embodiment 38, wherein Rₐ and R_{b} are each independently an HTM.
45. The compound of embodiment 26, wherein Rₐ and R_{b} combine with nitrogen on the same side of Formula one to form a 3-member to 8-member first heterocyclic ring substituted with an HTM, and wherein R_{c} and R_{d} combine with nitrogen on the same side of Formula one to form a second 3-member to 8-member heterocyclic ring substituted with an HTM.
46. The compound of embodiment 45, wherein the first and second heterocyclic rings are each independently a 5-member to 7-member ring.
47. The compound of embodiment 45, wherein the first and second heterocyclic rings are each independently a 6-member ring.
48. The compound of embodiment 45, wherein the first and second heterocyclic rings are each independently a diazinane ring.
49. The compound of embodiment 48, wherein the diazinane ring is a 1,4-diazinane ring.
50. The compound of embodiment 49, wherein the 1,4-diazinane ring is substituted with the HTM at the 4 position.
51. The compound of embodiment 1-50, wherein HTM is a C₅₋₄₀ linear or branched alkyl, C₅₋₄₀ linear or branched alkenyl, or C₅₋₄₀ linear or branched alkynyl.
52. The compound of embodiment 51, wherein HTM is a C₅₋₄₀ linear or branched alkyl.
53. The compound of embodiment 51, wherein HTM is a C₅₋₃₀ linear alkyl.
54. The compound of embodiment 51, wherein HTM is a C₅₋₂₀ linear alkyl.
55. The compound of embodiment 51, wherein HTM is a C₈₋₂₀ linear alkyl.
56. The compound of embodiment 51, wherein HTM is a C₁₀₋₂₀ linear alkyl.
57. The compound of embodiment 51, wherein HTM is a C₁₂₋₂₀ linear alkyl.
58. The compound of embodiment 51, wherein HTM is a C₁₄₋₂₀ linear alkyl.
59. The compound of embodiment 51, wherein HTM is a C₁₆₋₂₀ linear alkyl.
60. The compound of embodiment 51, wherein HTM is a C₁₆₋₁₈ linear alkyl.
61. The compound of embodiment 51-60, wherein HTM is a C₁₀₋₃₀ branched alkyl.
62. The compound of embodiment 61, wherein the branched alkyl has one C₅₋₁₅ alkyl branch on a C₅₋₁₅ alkyl main chain.
63. The compound of embodiment 51-60, wherein HTM is a C₁₅₋₄₀ branched alkyl.
64. The compound of embodiment 63, wherein the branched alkyl has two C₅₋₁₅ alkyl branches on a C₅₋₁₅ alkyl main chain.
65. The compound of embodiment 51-64, wherein the linear or branched alkyl, alkenyl, or alkynyl has one or more intervening groups selected from ester —(C═O)—O—, amide —(C═O)—NR—, or carboxylate —O—(C═O)—O—, wherein R is hydrogen, a C₁₋₄ alkyl, C₁₋₄ alkenyl, or C₁₋₄ alkynyl.
66. The compound of embodiment 65, wherein one or more intervening groups is selected from ester —(C═O)—O— or amide —(C═O)—NR—.
67. The compound of embodiment 66, wherein the one or more intervening group is inserted in the alkyl such that the acyl group is connect to nitrogen in Formula I or Formula II through an ethylene —CH2—CH2—
68. The compound of 67, wherein the one or more intervening group is one or more ester group.
69. The compounds of embodiment 1-68, wherein HTM has the structure of

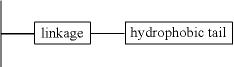

wherein the linkage is selected from

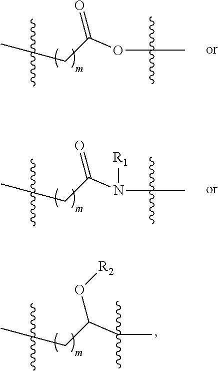

m is an integer of 1-6; R₁ is H, CH₃, or CH₂CH₃; and R₂ is H, CH₃, CH₂CH₃, or (C═O)CH₃, wherein the hydrophobic tail is selected from the group consisting of:

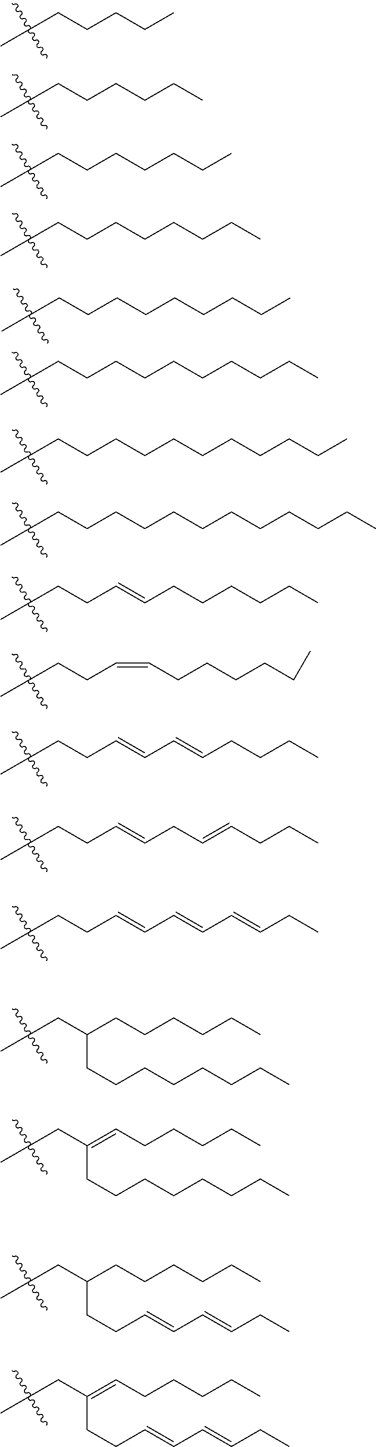

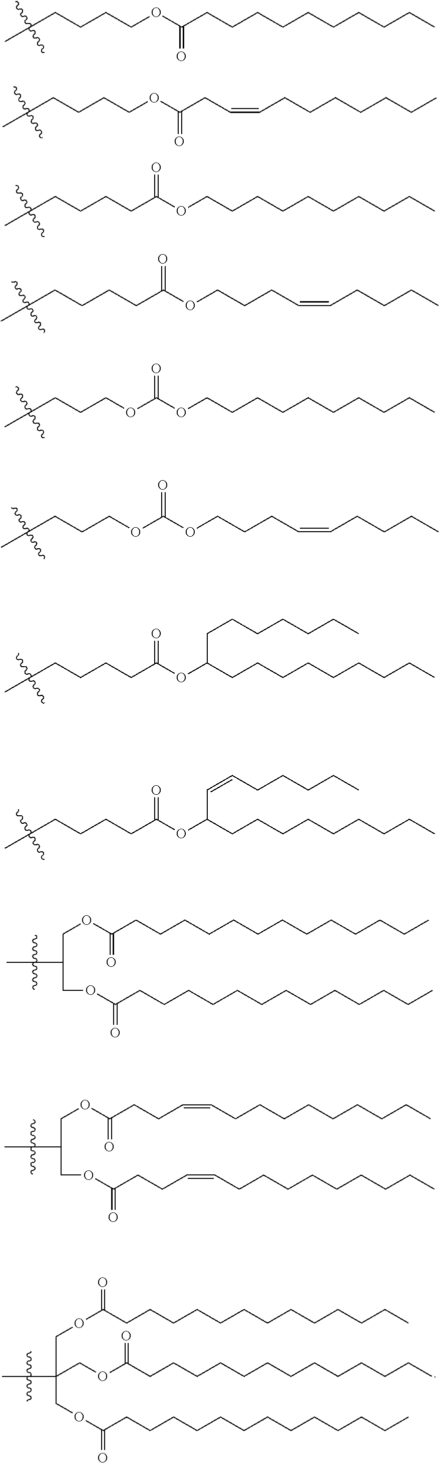

70. The compound of embodiment 1, selected from the group consisting of:

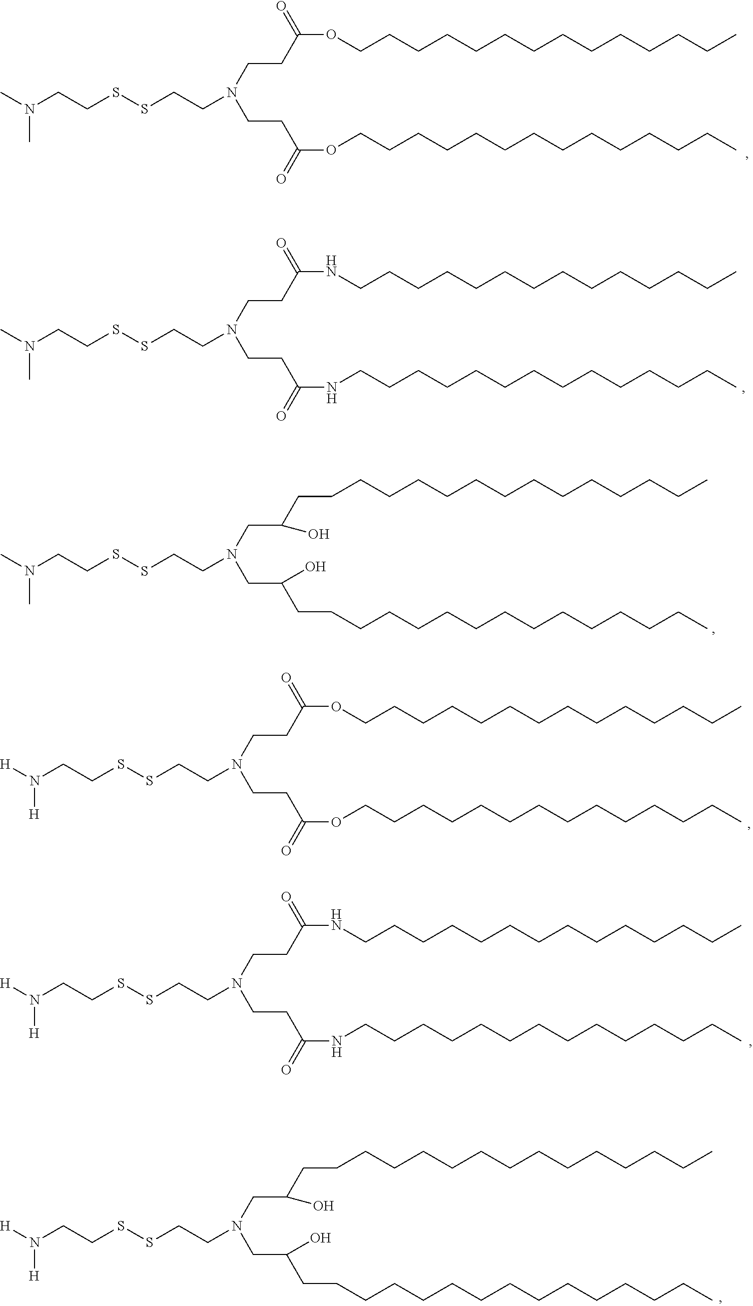

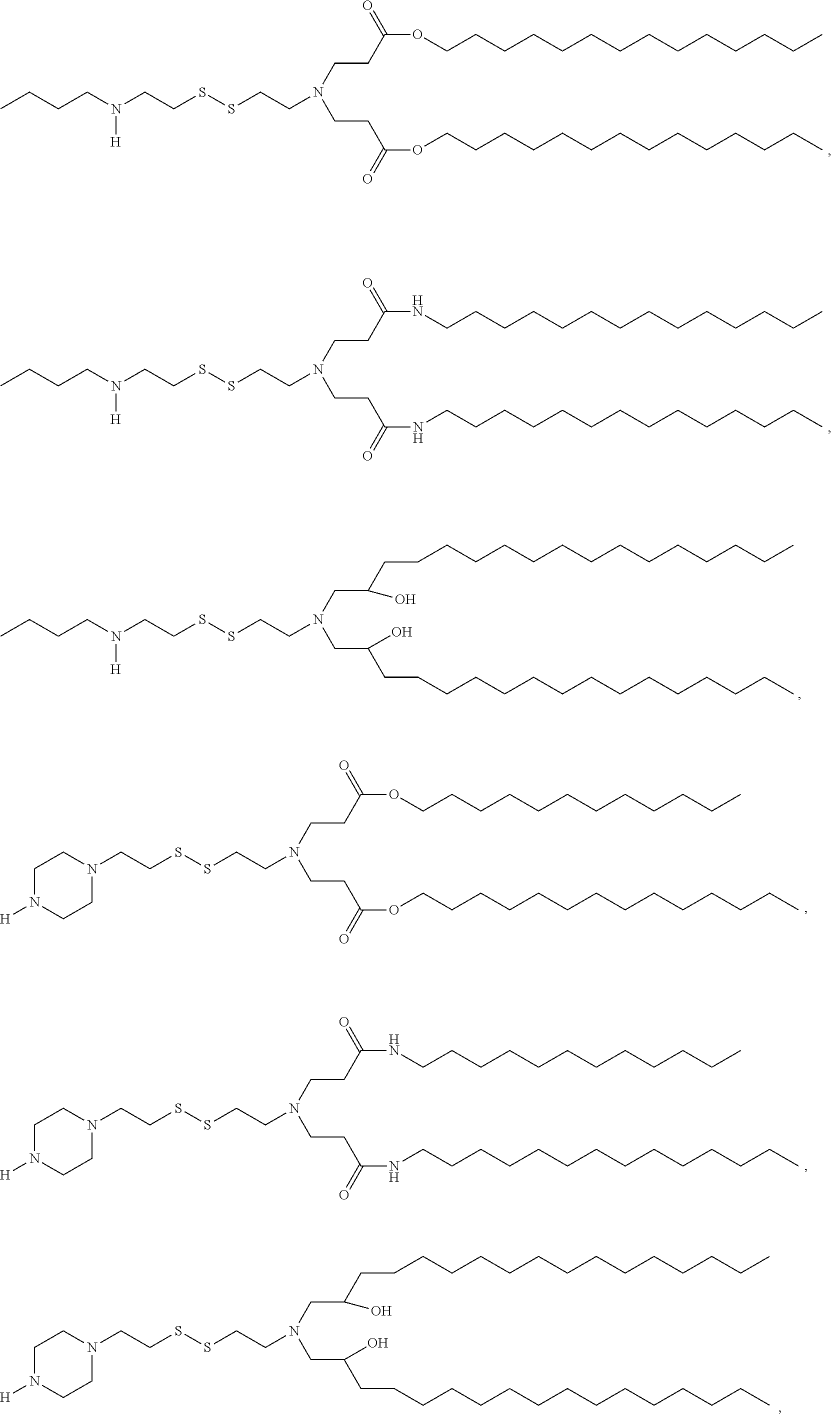

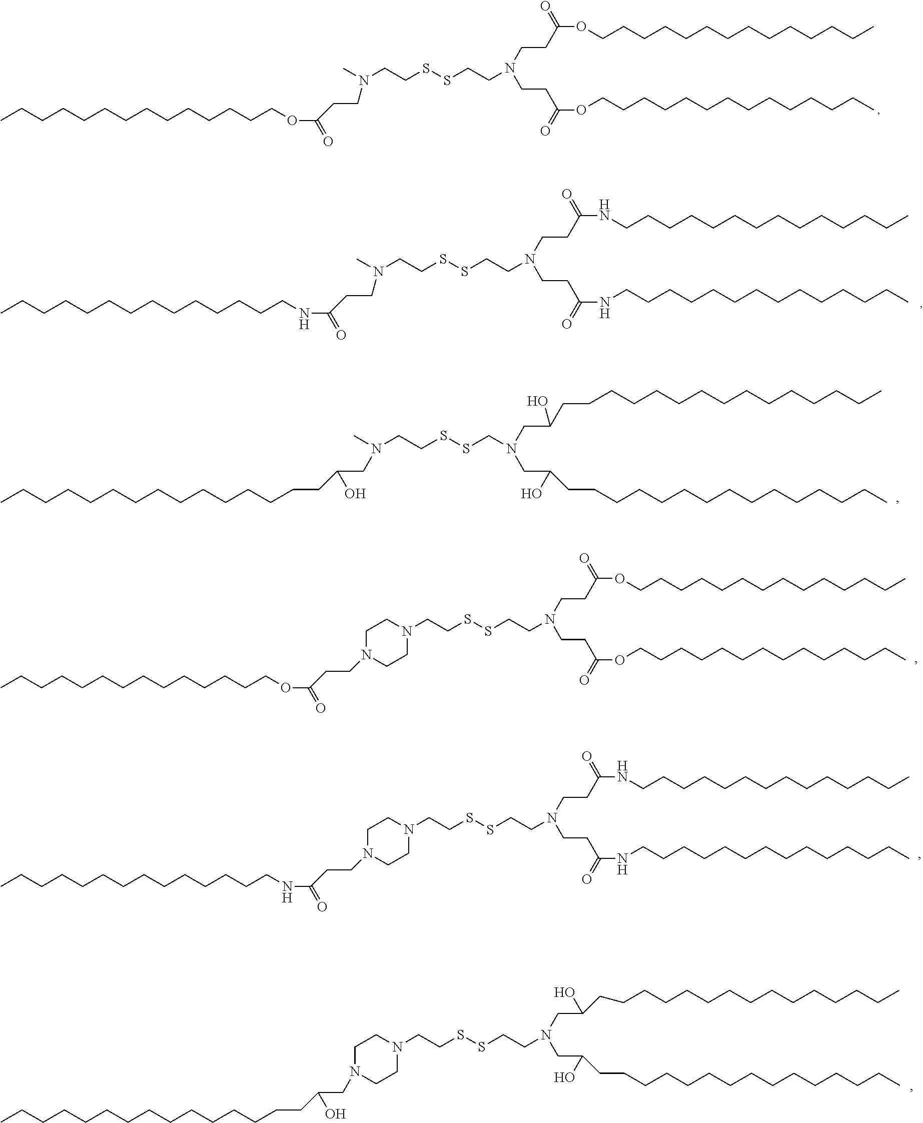

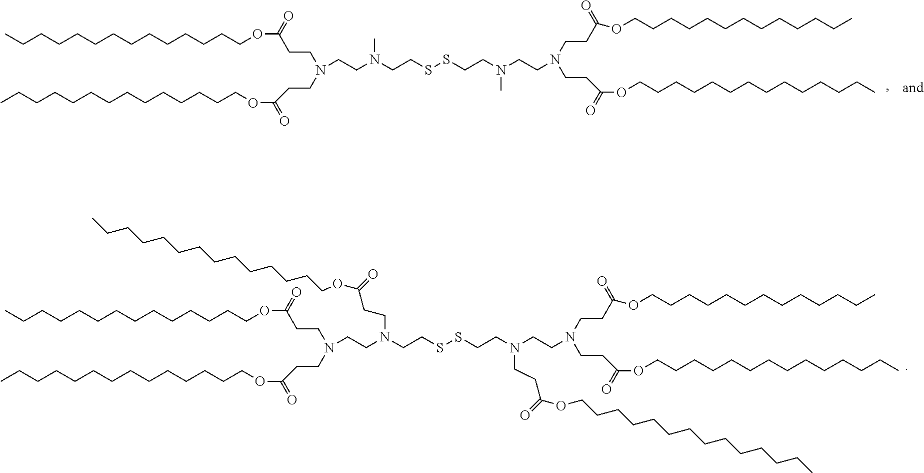

71. A compound of Formula III, a stereoisomer or pharmaceutically acceptable salt thereof:

(RₐR_{b}N-L₁-S—S-L₂-)nMVC (Formula III)

wherein MVC is a multi-valent core capable of connecting with two or more (RₐR_{b}N-L₁-S—S-L₂-), wherein n is an integer of 2-12;

wherein L₁ and L₂ are each independently C₁₋₄ alkylenyl;

wherein at least one of Rₐ and R_{b} is independently a hydrophobic tail moiety (HTM) or Rₐ and R_{b} combined with nitrogen to form a 3-member to 8-member heterocyclic ring substituted with an HTM, and the wherein the remainder of Rₐ, R_{b}, R_{c} and R_{d} is independently a hydrogen, a C₁₋₄ alkyl, or combine with nitrogen on the same side of Formula I to form a 3-member to 8-member heterocyclic ring optionally substituted with a C₁₋₄ alkyl,

wherein HTM is a C₅₋₄₀ linear or branched alkyl, C₅₋₄₀ linear or branched alkenyl, or C₅₋₄₀ linear or branched alkynyl; or wherein HTM has a structure of Formula II:

-L-NR′R″ (Formula II)

wherein L is C₁₋₄ alkylenyl and at least one of R′ and R″ is a C₅₋₄₅ linear or branched alkyl, C₅₋₄₅ linear or branched alkenyl, or C₅₋₄₅ linear or branched alkynyl.
72. The compound of embodiment 71, wherein L₁ and L₂ are each independently C₁₋₃ alkylenyl.
73. The compound of embodiment 72, wherein L1 is ethylene.
74. The compound of embodiment 72-73, wherein L2 is ethylene.
75. The compound of embodiment 71-74, wherein the MVC is a multivalent non-metal element.
76. The compound of embodiment 71-74, wherein the non-metal element is oxygen or nitrogen.
77. The compound of embodiment 71-74, wherein the non-metal element is nitrogen.
78. The compound of embodiment 71-74, wherein n is 2 and wherein the MVC is a divalent nitrogen —NR₁—, wherein R₁ is a hydrogen, a C₁₋₄ alkyl, or an HTM.
79. The compound of embodiment 78, wherein the R₁ is a C₁₋₄ alkyl.
80. The compound of embodiment 78, wherein the R₁ is a methyl.
81. The compound of clam 78, wherein the R₁ is HTM.
82. The compound of embodiment 78, wherein n is 3 and wherein the MVC is a trivalent nitrogen.
83. The compound of embodiment 75-82, wherein Rₐ is an HTM and R_{b} is a hydrogen or a C₁₋₄ alkyl.
84. The compound of embodiment 75-81, wherein Rₐ and R_{b} are each independently an HTM.
85. The compound of embodiment 75-81, wherein Rₐ and R_{b} combined with nitrogen to form a 3-member to 8-member heterocyclic ring substituted with an HTM.
86. The compound of embodiment 85, wherein the heterocyclic ring is a 5-member to 7-member ring.
87. The compound of embodiment 85, wherein the heterocyclic ring is a 6-member ring.
88. The compound of embodiment 85, wherein the heterocyclic ring is a diazinane ring.
89. The compound of embodiment 85, wherein the diazinane ring is a 1,4-diazinane ring.
90. The compound of embodiment 89, wherein the 1,4-diazinane ring is substituted with the HTM at the 4 position.
91. The compound of embodiment 71-74, wherein the MVC is a multivalent radical of an organic compound having multiple functional groups, and optionally wherein the organic compound has a molecular weight of no more than 300, no more than 200 or no more than 150.
92. The compound of 91, wherein the functional groups are selected from —C(═O)—O—, —O—C(═O)—, —C(═O)—NR₂—, —NR₂—C(═O)—, or combinations thereof, wherein R₂ is hydrogen, a C₁₋₄ alkyl, C₁₋₄ alkenyl, or C₁₋₄ alkynyl.
93. The compound of embodiment 92, wherein the functional groups are —C(═O)—O—.
94. The compound of embodiment 92, wherein the functional groups are —O—C(═O)—.
95. The compound of embodiment 92, wherein the functional groups are —C(═O)—NR₂—.
96. The compound of embodiment 92, wherein the functional groups are —NR₂—C(═O)—.
97. The compound of embodiment 91-96, wherein the organic compound is a nonaromatic compound.
98. The compound of embodiment 97, wherein the nonaromatic compound is an aliphatic compound.

199. The compound of embodiment 97, wherein the nonaromatic compound is a cyclic compound.
100. The compound of embodiment 97, wherein the cyclic nonaromatic compound is a heterocyclic compound.
101. The compound of embodiment 97, wherein the cyclic nonaromatic compound is a homocyclic compound.
102. The compound of embodiment 97-101, wherein the organic compound is a C₃₋₁₅ nonaromatic compound.
103. The compound of embodiment 91-96, wherein the organic compound is an aromatic compound.
104. The compound of embodiment 103, wherein the aromatic compound is an homoaromatic compound.
105. The compound of embodiment 103, wherein the aromatic compound is a heteroaromatic compound.
106. The compound of embodiment 103, wherein the aromatic compound is a heterocyclic compound.
107. The compound of embodiment 103-106, wherein the aromatic compound is a monocyclic aromatic compound.

1108. The compound of embodiment 103-106, wherein the aromatic compound is a polycyclic aromatic compound.
109. The compound of embodiment 103-108, wherein the organic compound is a C₅₋₁₅ aromatic compound.
110. The compound of embodiment 91-109, wherein the MVC is a divalent radical of the organic compound.
111. The compound of embodiment 91-109, wherein the MVC is a trivalent radical of the organic compound.
112. The compound of embodiment 91-109, wherein the MVC is a tetravalent radical of the organic compound.
113. The compound of embodiment 91-112, wherein Rₐ is an HTM and R_{b} is a hydrogen or a C₁₋₄ alkyl.
114. The compound of embodiment 91-112, wherein Rₐ and R_{b} are each independently an HTM.
115. The compound of embodiment 91-112, wherein Rₐ and R_{b} combined with nitrogen to form a 3-member to 8-member heterocyclic ring substituted with an HTM.
116. The compound of embodiment 115, wherein the heterocyclic ring is a 5-member to 7-member ring.
117. The compound of embodiment 115, wherein the heterocyclic ring is a 6-member ring.
118. The compound of embodiment 115, wherein the heterocyclic ring is a diazinane ring.
119. The compound of embodiment 115, wherein the diazinane ring is a 1,4-diazinane ring.
120. The compound of embodiment 119, wherein the 1,4-diazinane ring is substituted with the HTM at the 4 position.
121. The compound of embodiment 91-119, wherein the MVC is selected from:

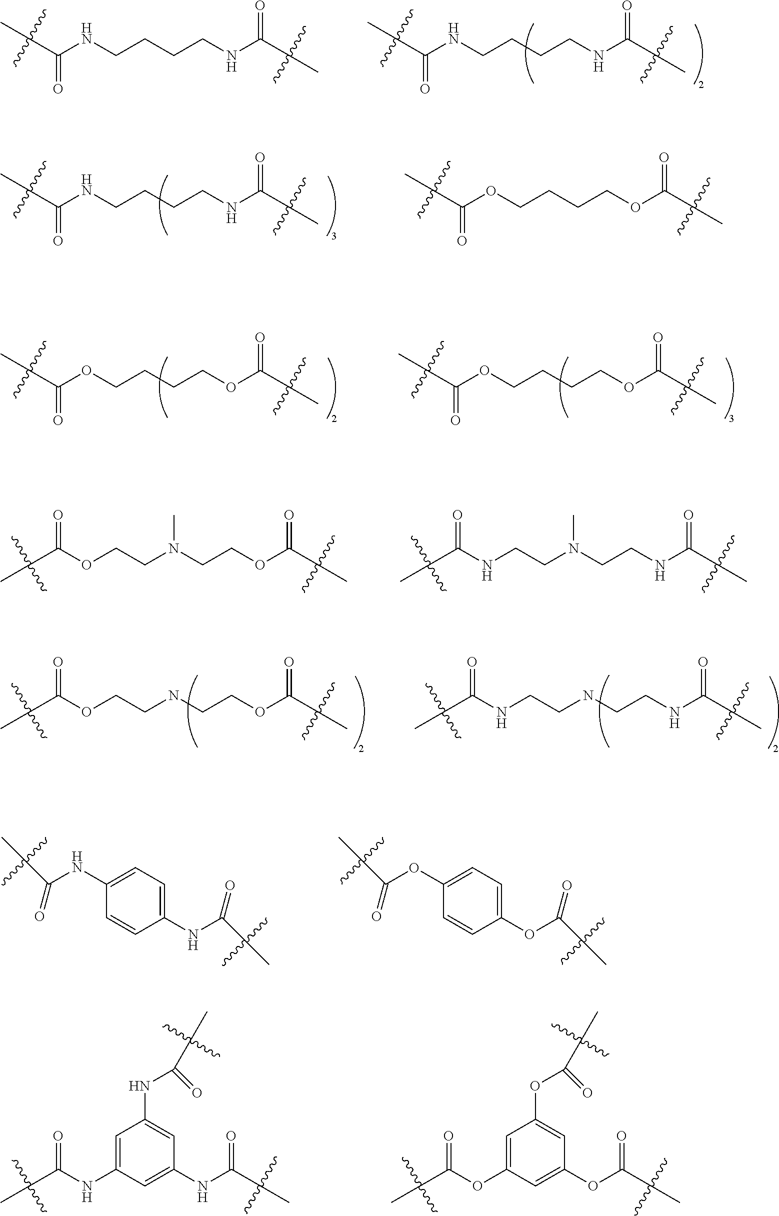

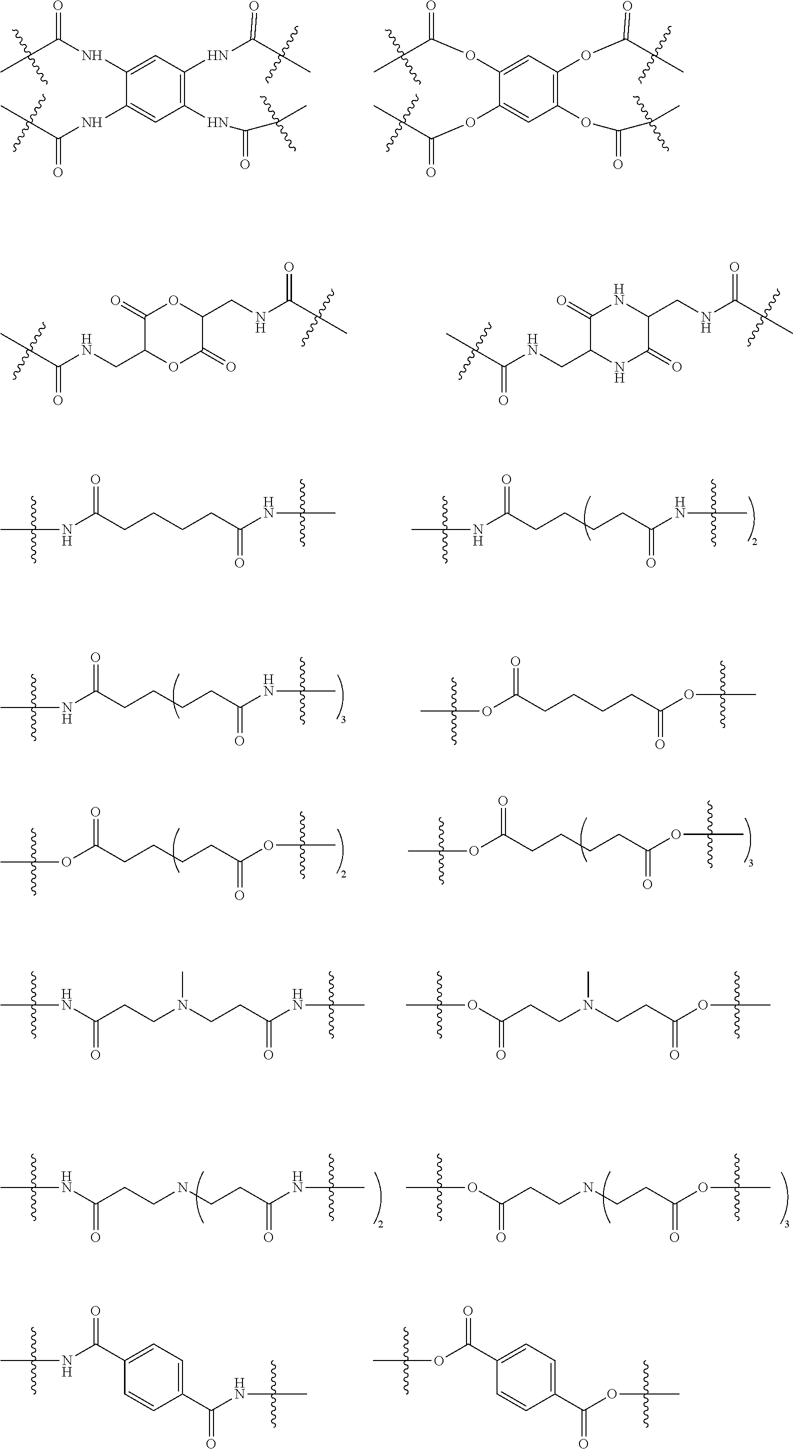

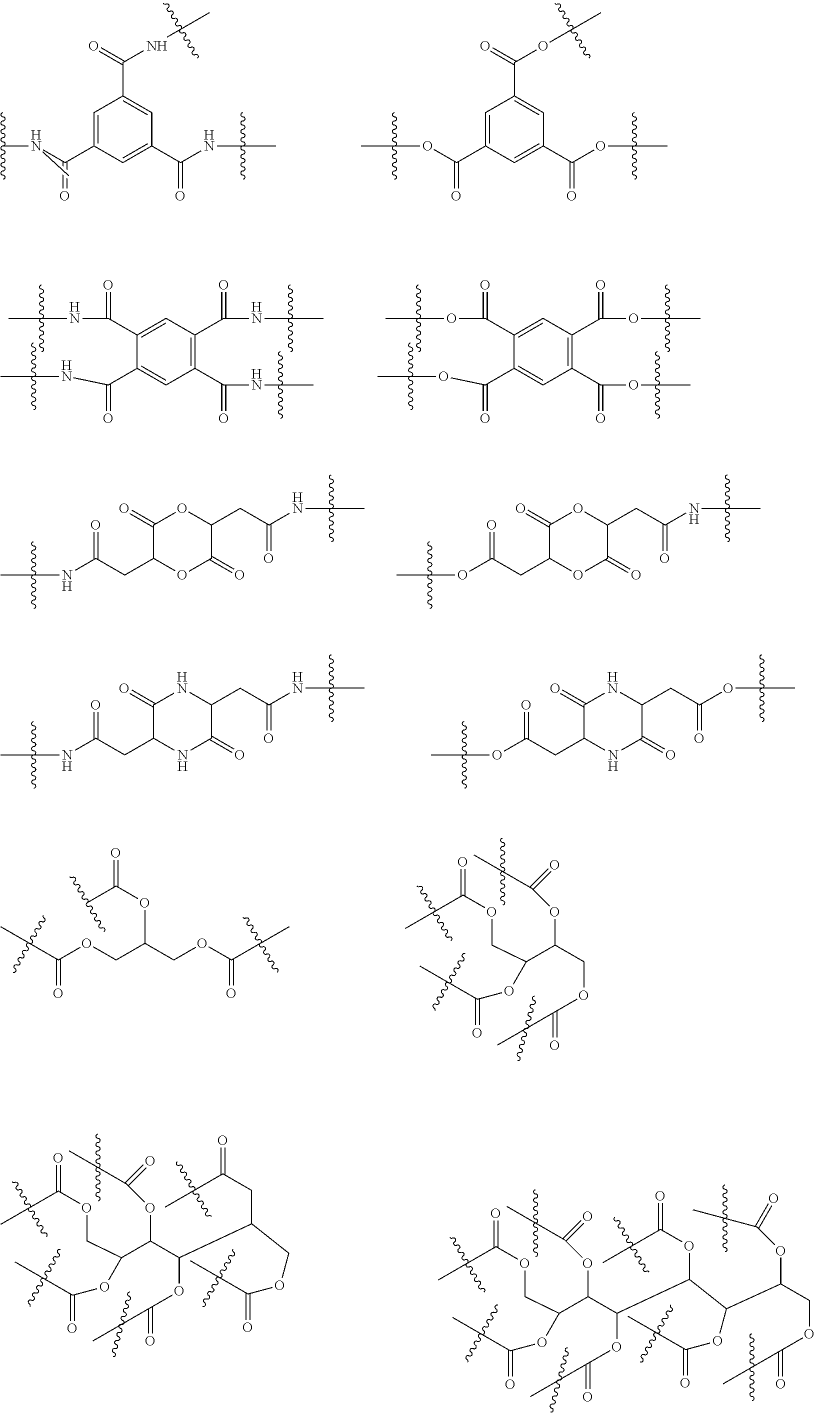

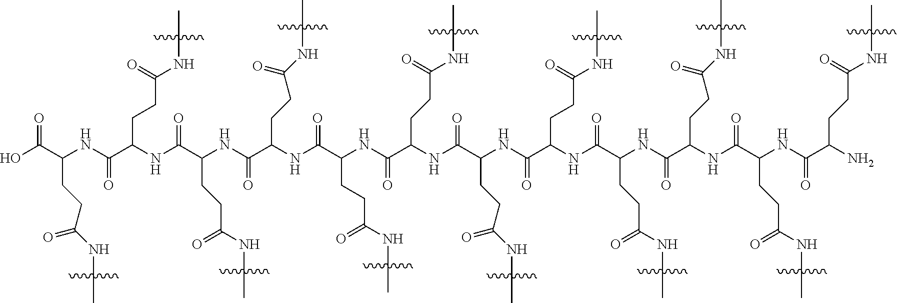 [SMILES: *NC(=O)CCC(N)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)NC(CCC(=O)N*)C(=O)O]

122. The compound of embodiment 71-121, wherein HTM is a C₅₋₄₀ linear or branched alkyl, C₅₋₄₀ linear or branched alkenyl, or C₅₋₄₀ linear or branched alkynyl.
123. The compound of embodiment 122, wherein HTM is a C₅₋₄₀ linear or branched alkyl.
124. The compound of embodiment 123, wherein HTM is a C₅₋₃₀ linear alkyl.
125. The compound of embodiment 123, wherein HTM is a C₅₋₂₀ linear alkyl.
126. The compound of embodiment 123, wherein HTM is a C₈₋₂₀ linear alkyl.
127. The compound of embodiment 123, wherein HTM is a C₁₀₋₂₀ linear alkyl.
128. The compound of embodiment 123, wherein HTM is a C₁₂₋₂₀ linear alkyl.
129. The compound of embodiment 123, wherein HTM is a C₁₄₋₂₀ linear alkyl.
130. The compound of embodiment 123, wherein HTM is a C₁₆₋₂₀ linear alkyl.
131. The compound of embodiment 123, wherein HTM is a C₁₆₋₁₈ linear alkyl.
132. The compound of embodiment 123, wherein HTM is a C₁₀₋₃₀ branched alkyl.
133. The compound of embodiment 132, wherein the branched alkyl has one C₅₋₁₅ alkyl branch on a C₅₋₁₅ alkyl main chain.
134. The compound of embodiment 123, wherein HTM is a C₁₅₋₄₀ branched alkyl.
135. The compound of embodiment 134, wherein the branched alkyl has two C₅₋₁₅ alkyl branches on a C₅₋₁₅ alkyl main chain.
136. The compound of embodiment 122-135, wherein the linear or branched alkyl, alkenyl, or alkynyl has one or more intervening groups selected from ester —(C═O)—O—, amide —(C═O)—NR—, or carboxylate —O—(C═O)—O—.
137. The compound of embodiment 136, wherein one or more intervening groups is selected from ester —(C═O)—O— or amide —(C═O)—NR—.
138. The compound of embodiment 137, wherein the one or more intervening group is inserted in the alkyl such that the acyl group is connect to nitrogen in Formula I or Formula II through an ethylene —CH₂—CH₂—.
139. The compound of 138, wherein the one or more intervening group is one or more ester group.
140. The compounds of embodiment 122-139, wherein HTM has the structure of

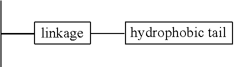

wherein the linkage is selected from

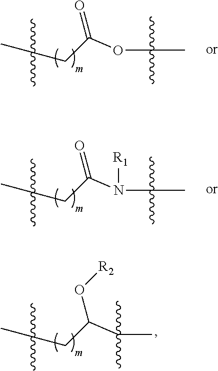

m is an integer of 1-6; R₁ is H, CH₃, or CH₂CH₃; and R₂ is H, CH₃, CH₂CH₃, or (C═O)CH₃, wherein the hydrophobic tail is selected from the group consisting of:

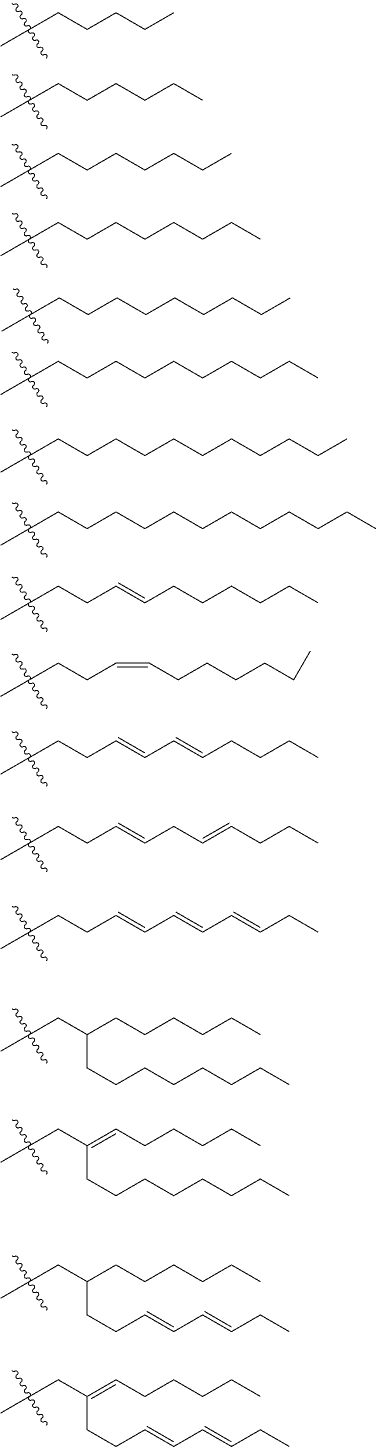

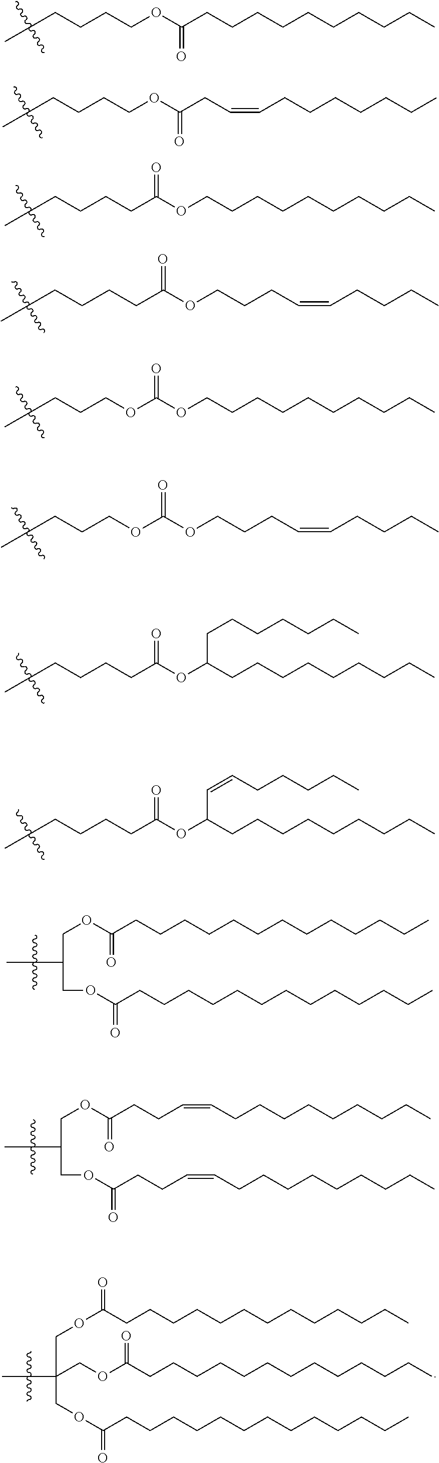

141. The compound of embodiment 71, selected from the group consisting of:

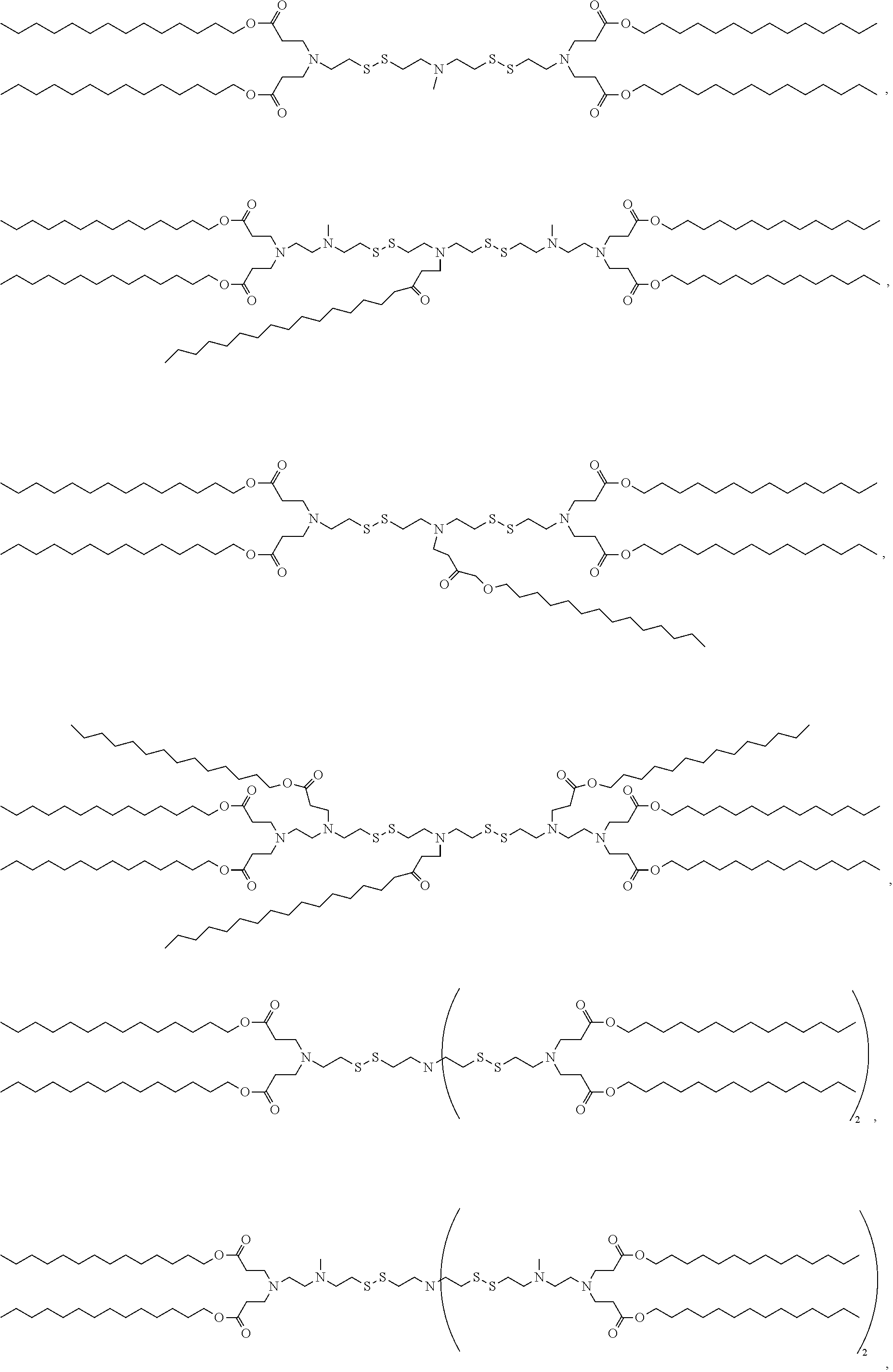

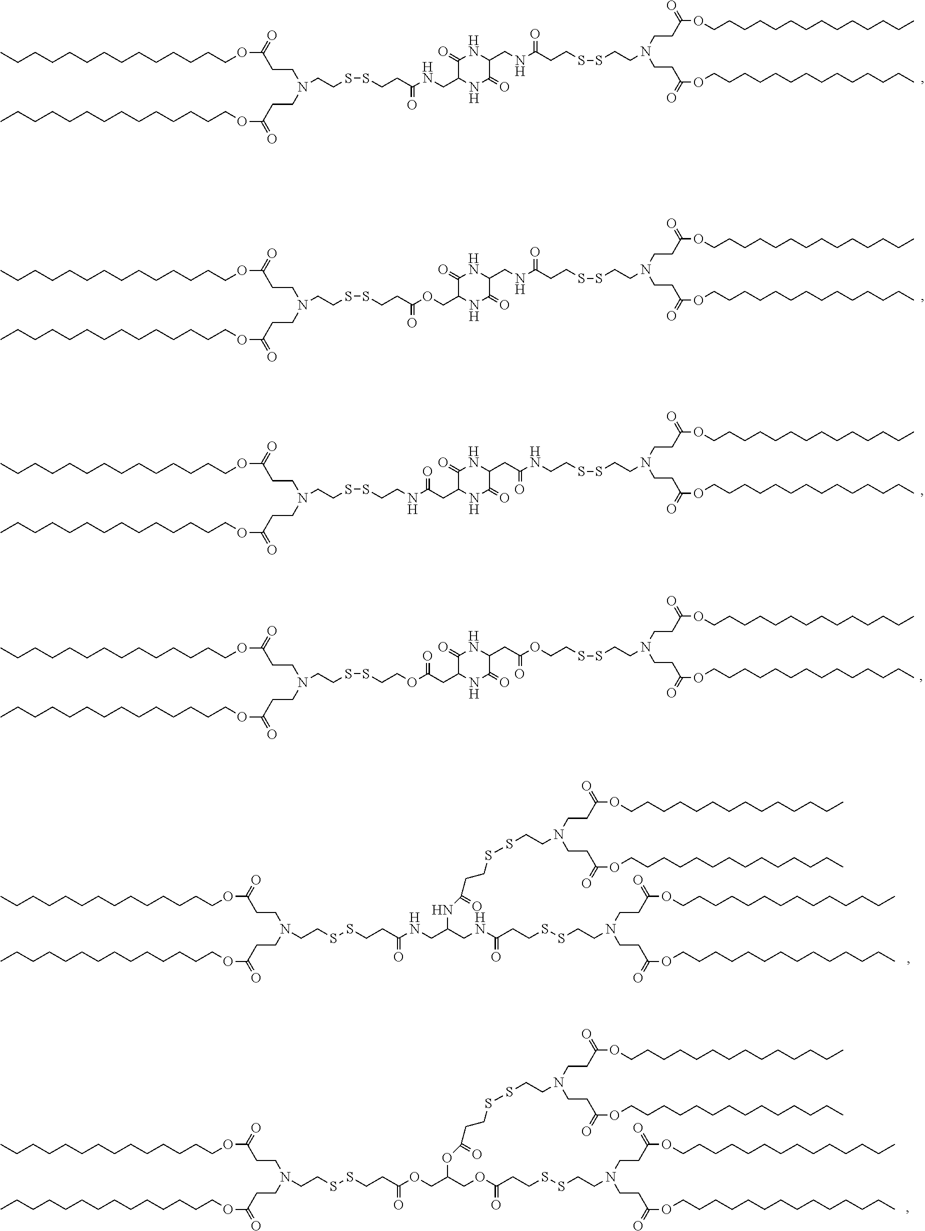

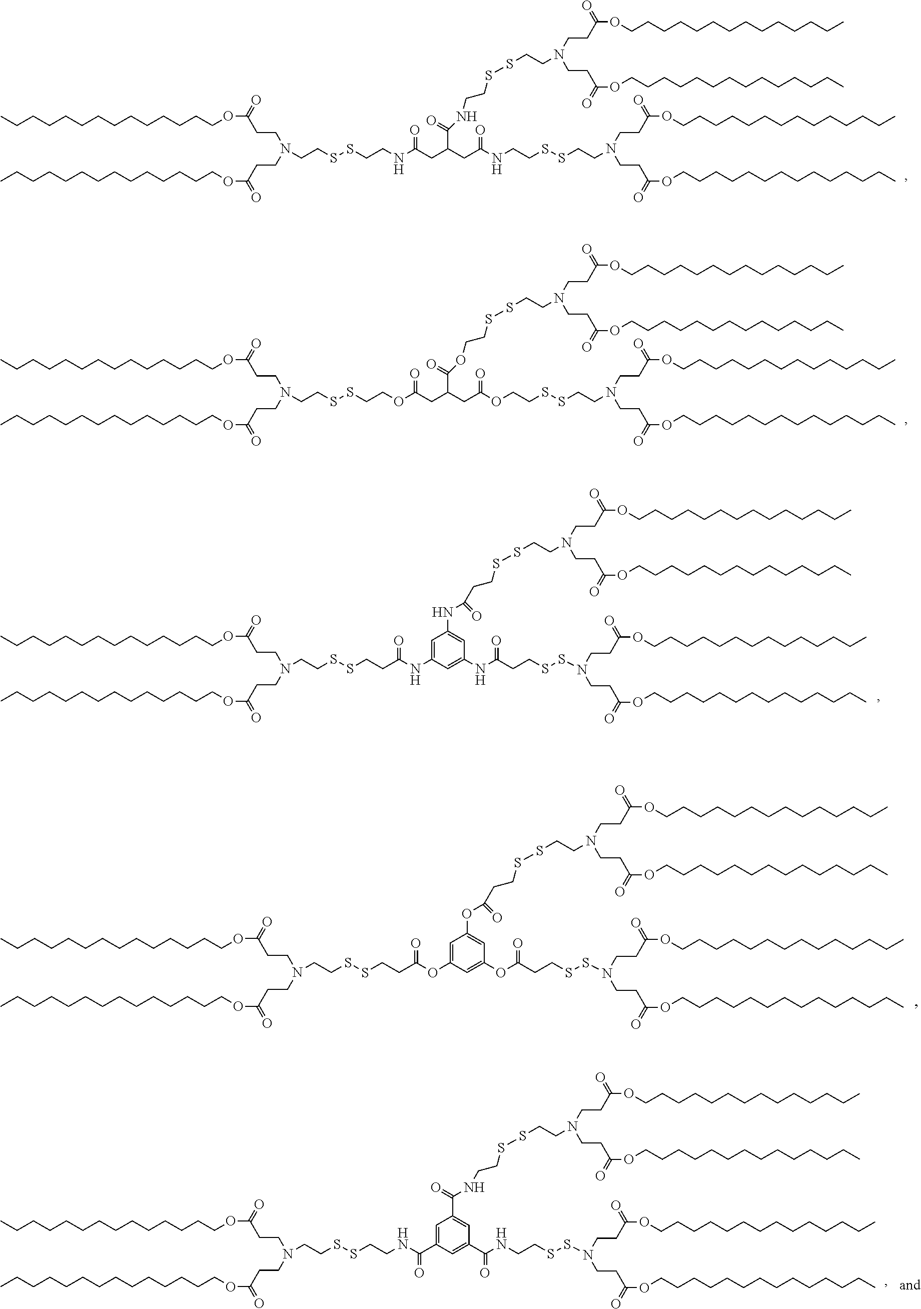

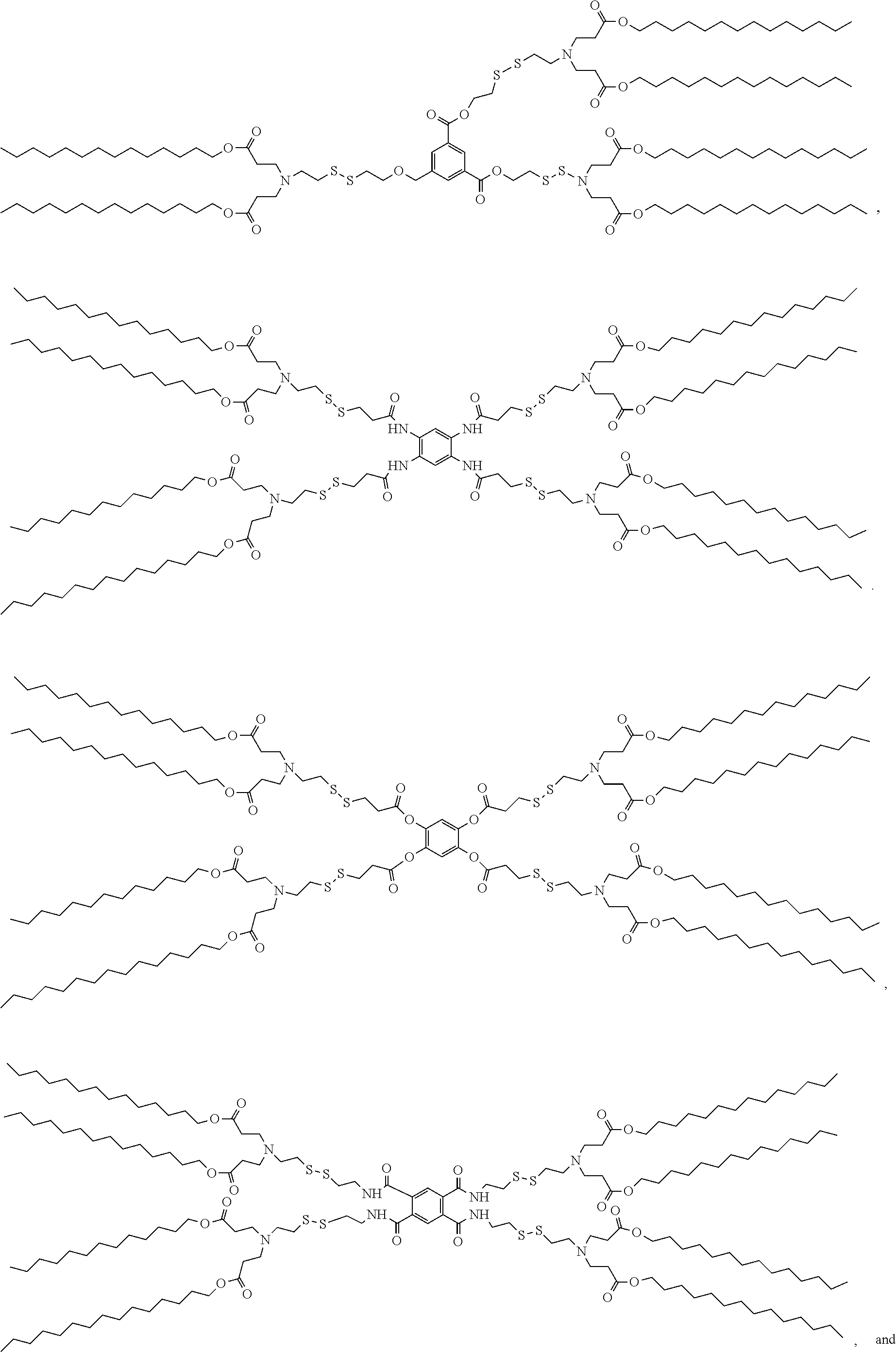

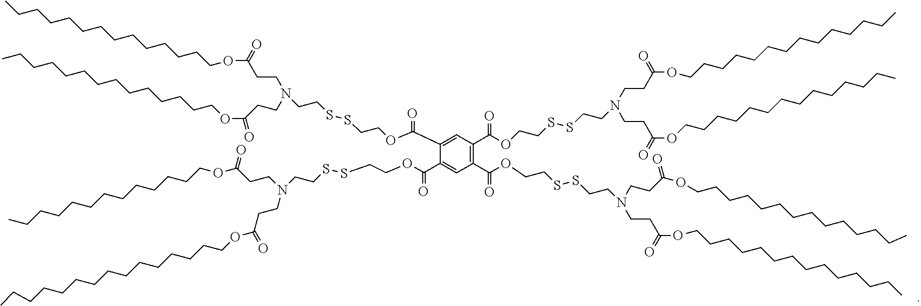 [SMILES: CCCCCCCCCCCCCCCOC(=O)CCN(CCSSCCOC(=O)c1cc(C(=O)OCCSSCCN(CCC(=O)OCCCCCCCCCCCCCC)CCC(=O)OCCCCCCCCCCCCCC)c(C(=O)OCCSSCCN(CCC(=O)OCCCCCCCCCCCCCC)CCC(=O)OCCCCCCCCCCCCCC)cc1C(=O)OCCSSCCN(CCC(=O)OCCCCCCCCCCCCCC)CCC(=O)OCCCCCCCCCCCCCC)CCC(=O)OCCCCCCCCCCCCC]

### NON-LIMITING EXAMPLES

The examples described herein are for illustrative purposes only and various modifications or changes suggested to persons skilled in the art are to be included within the spirit and purview of this application and scope of the appended claims. These examples are provided for illustrative purposes only and not to limit the scope of the claims provided herein.

### I. Chemical Synthesis

### List of Abbreviations

As used above, and throughout the disclosure, the following abbreviations, unless otherwise indicated, shall be understood to have the following meanings:
ACN or MeCN acetonitrile
Bn benzyl
BOC or Boc tent-butyl carbamate
t-Bu tert-butyl
Cy cyclohexyl
DBA dibenzylideneacetone
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
Dppf or dppf 1,1′-bis(diphenylphosphino)ferrocene
eq equivalent(s)
Et ethyl
Et₂O diethyl ether
EtOH ethanol
EtOAc ethyl acetate
HPLC high performance liquid chromatography
LAH lithium aluminum anhydride
LCMS liquid chromatography mass spectrometry
Me methyl
MeOH methanol
MS mass spectroscopy
NMM N-methyl-morpholine
NMP N-methyl-pyrrolidin-2-one
NMR nuclear magnetic resonance
RP-HPLC reverse phase-high pressure liquid chromatography
TFA trifluoroacetic acid
THF tetrahydrofuran
TLC thin layer chromatography

Unless otherwise noted, reagents and solvents were used as received from commercial suppliers. Anhydrous solvents and oven-dried glassware were used for synthetic transformations sensitive to moisture and/or oxygen. Yields were not optimized. Reaction times were approximate and were not optimized. Column chromatography and thin layer chromatography (TLC) were performed on silica gel unless otherwise noted.

### Example 1—Synthesis of Intermediate 1 (IM-1) CLLM-1

One example of synthesizing intermediates that can be used in preparation of the CDM compounds disclosed herein is provided below.

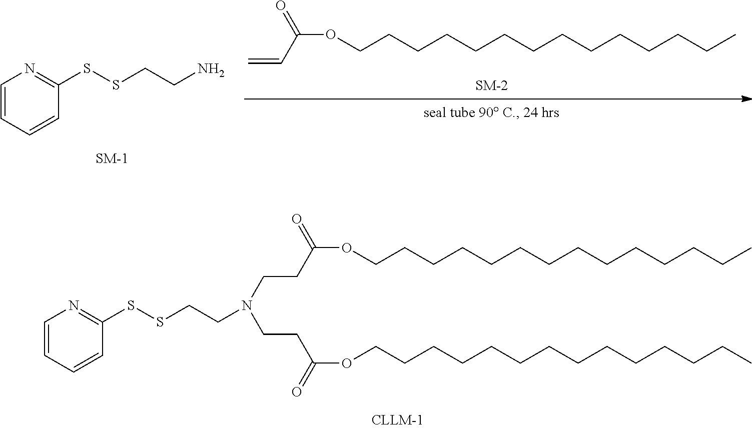

General procedure for the preparation of CLLM-1: A mixture of starting material 1 (SM-1, 300 mg, 1.6 mmol, 1.0 eq) and starting material 2 (SM-2, 1 mL, excess) in a 50-mL seal tube was stirred at 90 oC for 24 h. The reaction was monitored by TLC. The reaction solution was purified by flash column chromatography (FCC) to cleavable lipid-like material 1 (CLLM-1, 300 mg, 26%) as yellow oil.

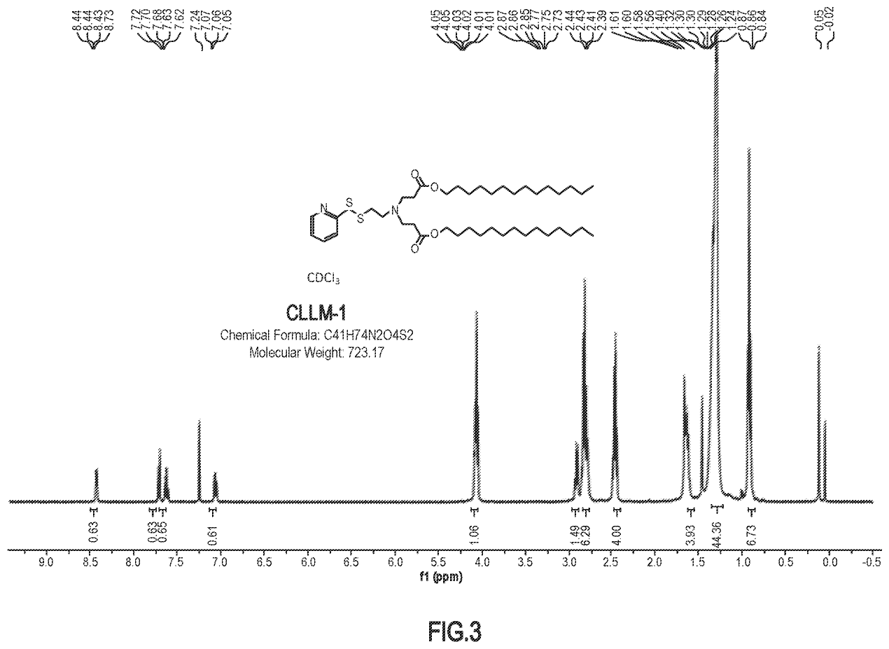

NMR characterization of CLLM-1 is summarized below and shown in FIG. 3.

¹H NMR (400 MHz, CDCl₃): δ8.4 (1, 1H), 7.7 (d, 1H), 7.6 (m, 1H), 7.1 (m, 1H), 4.0 (t, 4H), 2.87 (m, 2H), 2.77 (m, 6H), 2.42 (t, 4H), 1.59 (m, 4H), 1.31-1.23 (m, 44H), 0.86 (t, 6H).

### Example 2—Synthesis of Type I CDM Compound CLLM-2

One example of synthesizing a Type I CDM Compound CLLM-2 from the intermediate CLLM-1 in Example 1 is provided below. Similar procedures can be used to prepare Type II CDM compounds using SM-3 with appropriate HTM(s).

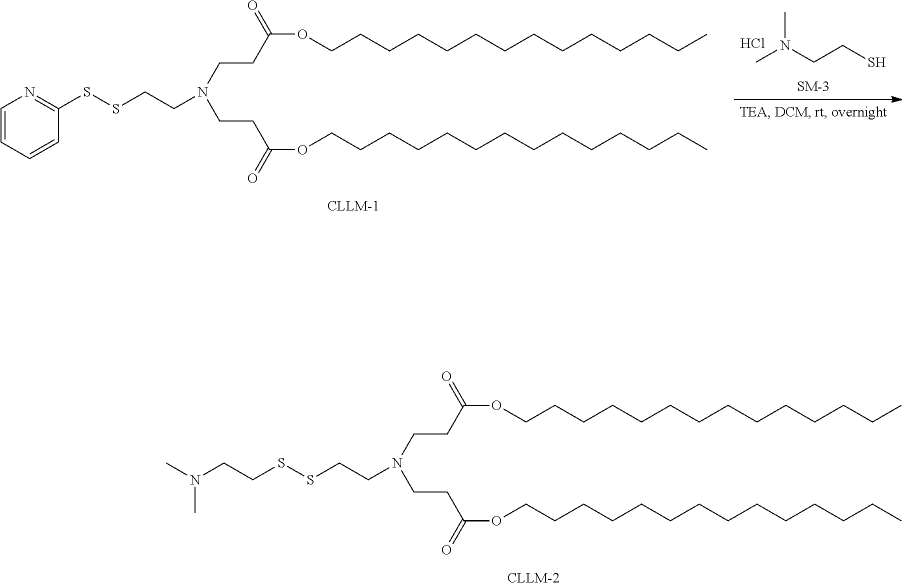

General procedure for the preparation of CLLM-2: A solution of CLLM-1 (1.0 g, 1.4 mmol, 1.0 eq) in DCM (20 mL) was added starting material 3 (SM-3, 296 mg, 2.1 mmol, 1.5 eq) and triethylamine (424 mg, 4.2 mmol, 3.0 eq). The reaction was stirred at rt overnight. The reaction was monitored by TLC. The solution was purified by FCC give cleavable lipid-like material 2 (CLLM-2, 140.8 mg, 14%) as yellow oil.

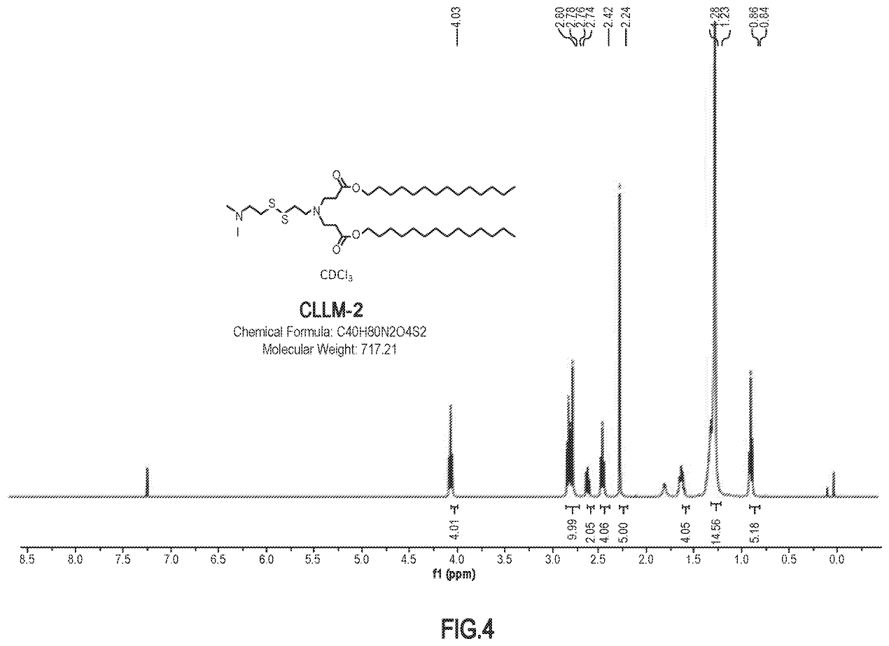

NMR characterization of CLLM-2 is summarized below and shown in FIG. 4.

¹HNMR (400 MHz, CDCl₃): δ4.0 (t, 4H), 2.80-2.74 (m, 10H), 2.58 (t, 2H), 2.42 (t, 4H), 2.24 (s, 6H), 1.59 (m, 4H), 1.31-1.23 (m, 44H), 0.86 (t, 6H).

### Example 3—Synthesis of Intermediate 2 (IM-2) CLLM-3

One example of synthesizing intermediates CLLM-3 that can be used in preparation of CDM compounds disclosed herein is provided below. While CLLM-3 is an intermediate in this example, CLLM-3 is a Type I CDM compound in some other embodiment of the present disclosure.

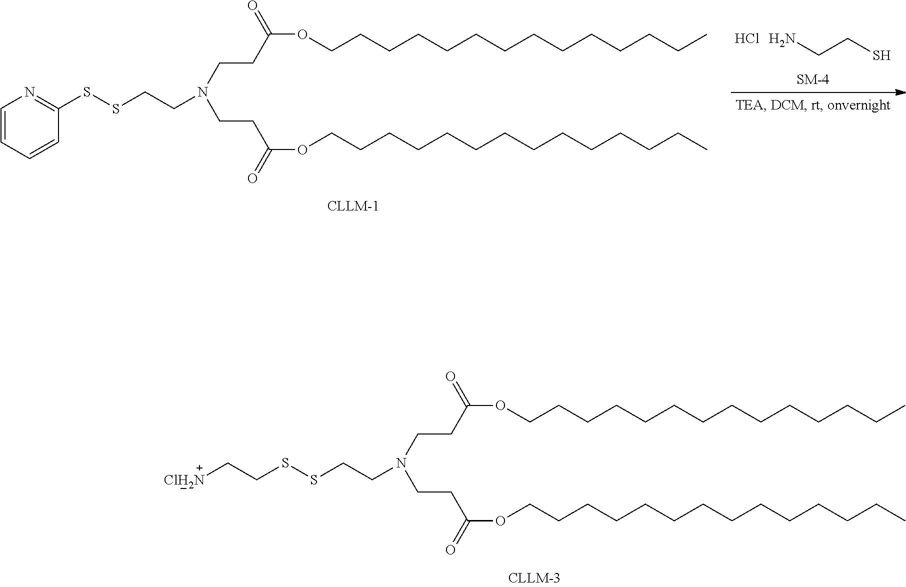

General procedure for the preparation of CLLM-3: A solution of CLLM-1 (1.0 g, 1.4 mmol, 1.0 eq) in DCM (20 mL) was added starting material 4 (SM-4, 237 mg, 2.1 mmol, 1.5 eq) and triethylamine (424 mg, 4.2 mmol, 3.0 eq). The reaction was stirred at rt overnight. The reaction was monitored by TLC. The solution was purified by FCC give cleavable lipid-like material 3 (CLLM-3, 140.8 mg, 14%) as yellow oil.

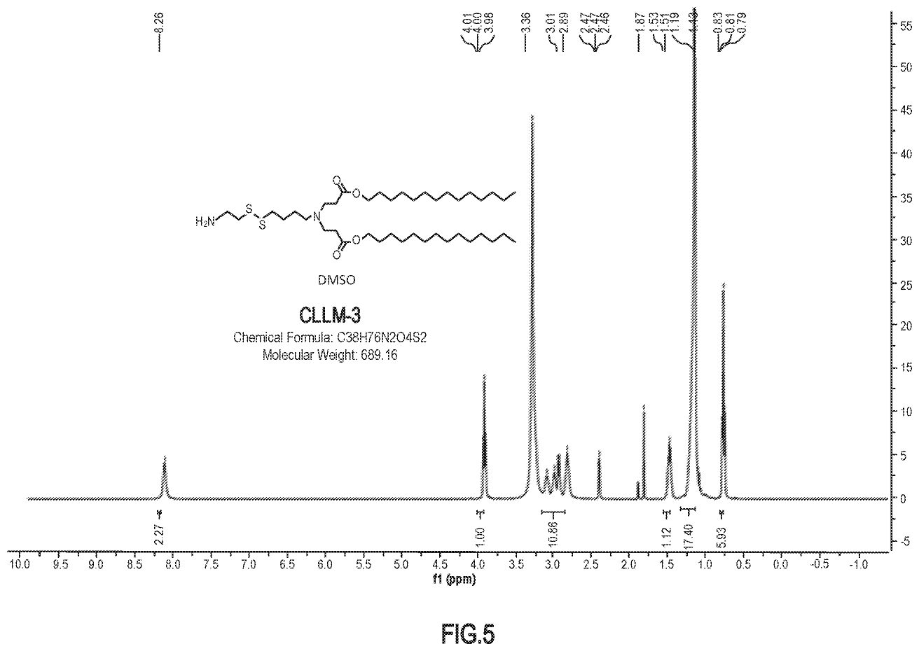

NMR characterization of CLLM-3 is summarized below and shown in FIG. 5.

¹HNMR (400 MHz, DMSO): δ8.26 (br, 2H), 4.0 (t, 4H), 3.01-2.89 (m, 10H), 1.59 (m, 4H), 1.19 (br, 44H), 0.81 (t, 6H).

### Example 4—Synthesis of Type IV CDM Compound CLLM-4

One example of synthesizing a Type IV CDM Compound CLLM-4 from the intermediate CLLM-3 in Example 3 is provided below.

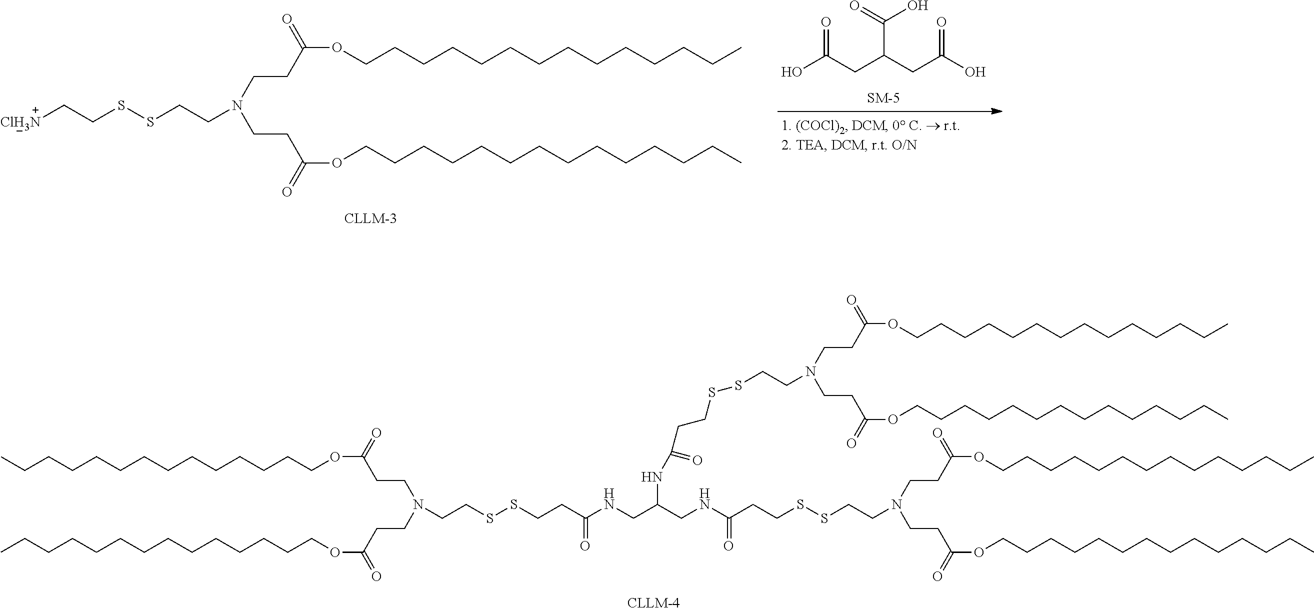

General procedure for the preparation of CLLM-3: To a solution of starting material 4 (SM-5, 500 mg, 2.84 mmol, 1.0 eq) in DCM (10 mL) at 0 oC was added (COCl)2 (3.6 g, 28.4 mmol, 10.0 eq) and DMF (30 mg). The reaction mixture was stirred at rt for 2 h. The mixture was concentrated under reduced pressure to remove the volatile. The residue was dissolved in DCM (5 mL), and used for next step.

To a solution of CLLM-3 (2.3 g, 3.47 mmol, 4.0 eq) in DCM (15 mL) 0 oC was added triethylamine (790 mg 7.8 mmol, 9.0 eq) and the acyl chloride solution prepared above. The reaction mixture was stirred at rt for 1 h. The reaction was monitored by TLC. Then the mixture was quenched with water (30 mL) and extracted with DCM (100 mL×2). The combined organic layers were washed with brine, and dried over sodium sulfate and filtered. The filtration was concentrated under reduced pressure. The residue was purified by FCC to give cleavable lipid-like material 4 (CLLM-4, 171 mg, 9%) as colorless oil.

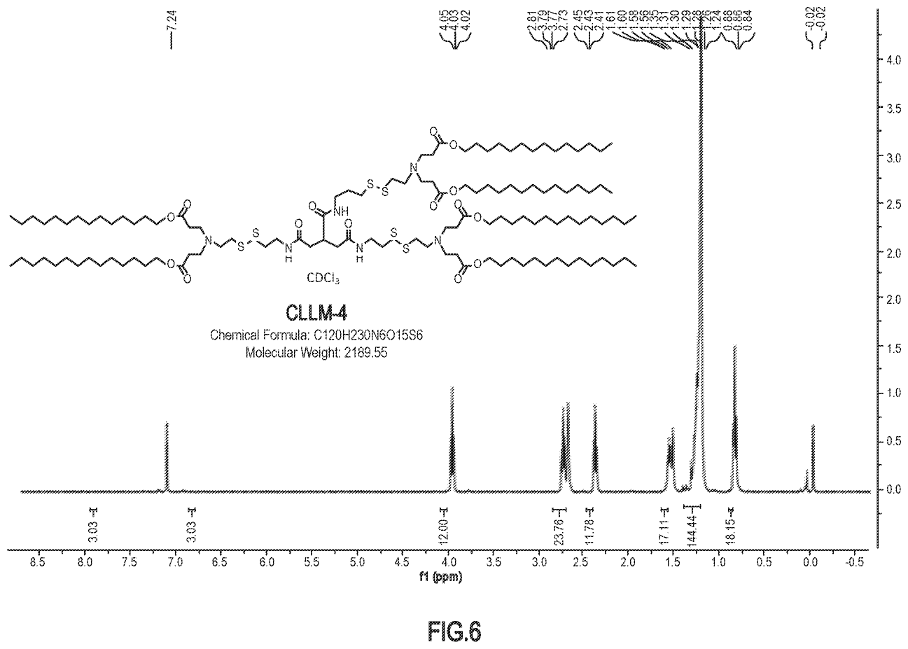

NMR characterization of CLLM-4 is summarized below and shown in FIG. 6.

¹HNMR (400 MHz, CDCl₃): δ4.05 (t, 12H), 2.81-2.73 (m, 24H), 2.45 (t, 12H), 1.61-1.56 (m, 17H), 1.35-1.24 (m, 144H), 0.88 (t, 18H)

### Example 5—Synthesis of Intermediate 2 (IM-2) CLLM-3 Without Disulfide Exchange Reaction

One example of synthesizing intermediates CLLM-3 without the disulfide exchange reaction is provided below.

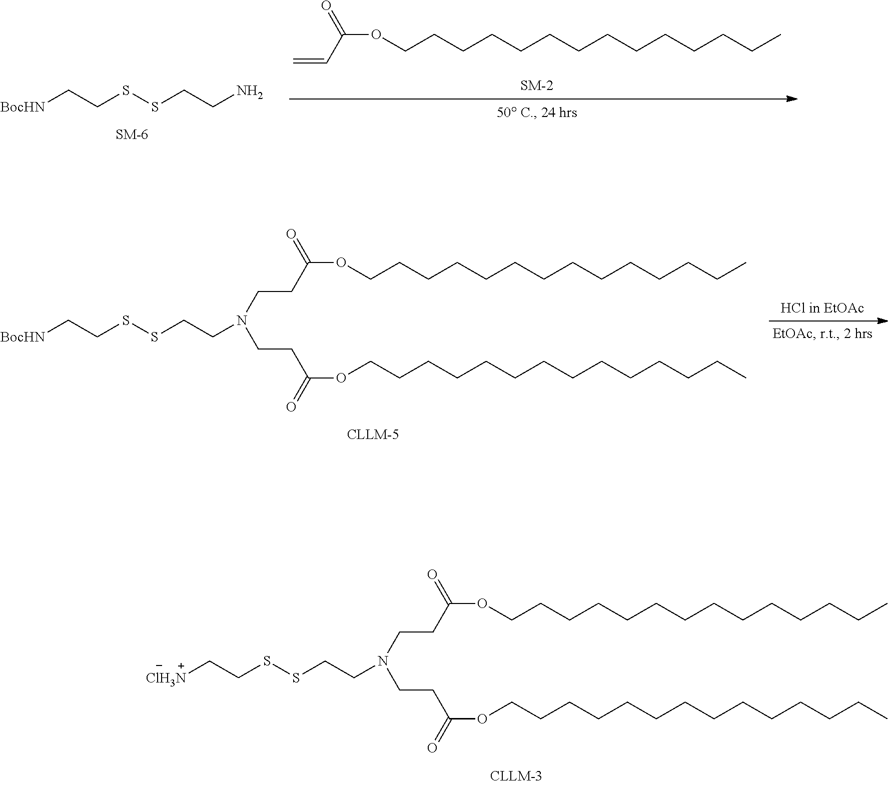

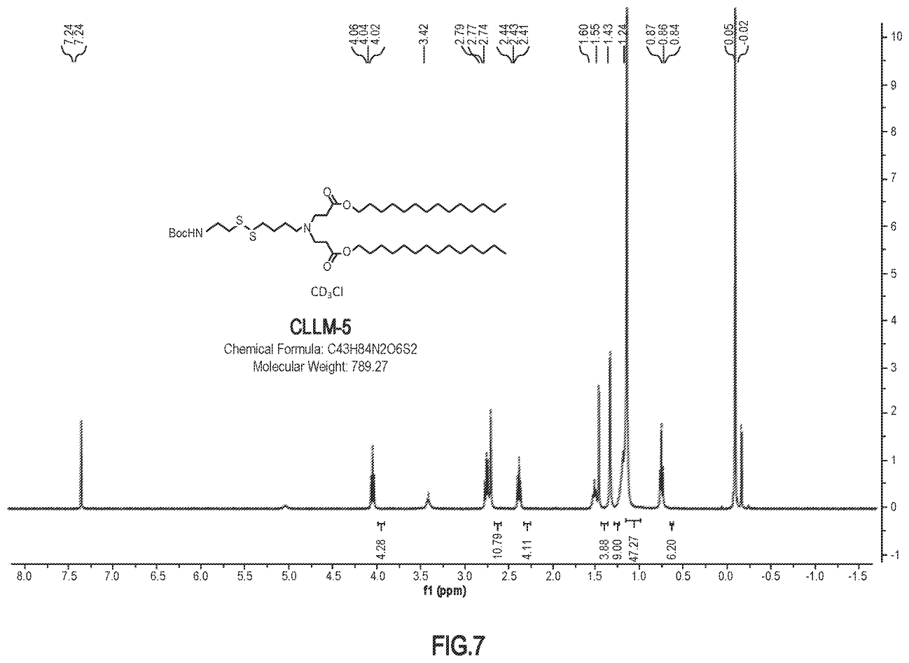

General procedure for the preparation of CLLM-5: A mixture of SM-6 (1.0 g, 4.0 mmol, 1.0 eq) and SM-2 (3.3 g, 12.0 mmol, 3.0 eq) was stirred at 50 oC for 24h. The reaction was monitored by TLC. Then the mixture was extracted with EA (100 mL×2). The organic phases were washed with brine, and dried over sodium sulfate and concentrated under reduced pressure. The residue was purified by FCC CLLM-5 (2.48 g, 80%) as white solid. NMR characterization of CLLM-5 is summarized below and shown in FIG. 7.

¹HNMR of CLLM-5 (400 MHz, CDCl₃): 4.04 (t, 4H), 3.42 (br, 1H), 2.79-2.74 (m, 10H), 2.45-2.42 (t, 4H), 1.60-1.55 (m, 4H), 1.43 (s, 9H),1.28-1.23 (m, 46H), 0.86 (t, 6H).

General procedure for the preparation of CLLM-3 from CLLM-5: CLLM-5 (2.48 g, 3.14 mmol, 1.0 eq) was treated with in HCl in ethyl acetate (4 N, 20 mL). The reaction mixture was stirred at rt for 2h. The reaction was monitored by TLC. The mixture was concentrated under reduced pressure. Ethyl acetate (50 mL) was added to the reaction mixture and vacuum-dried to afford CLLM-3 (2.35, 98%) as yellow solid.

NMR characterization of CLLM-3 is summarized in Example 3 and shown in FIG. 5

### Example 6—Synthesis of Type IV CDM Compound CLLM-6

One example of synthesizing a Type IV CDM Compound CLLM-6 from the intermediate CLLM-3 in Examples III and V is provided below.

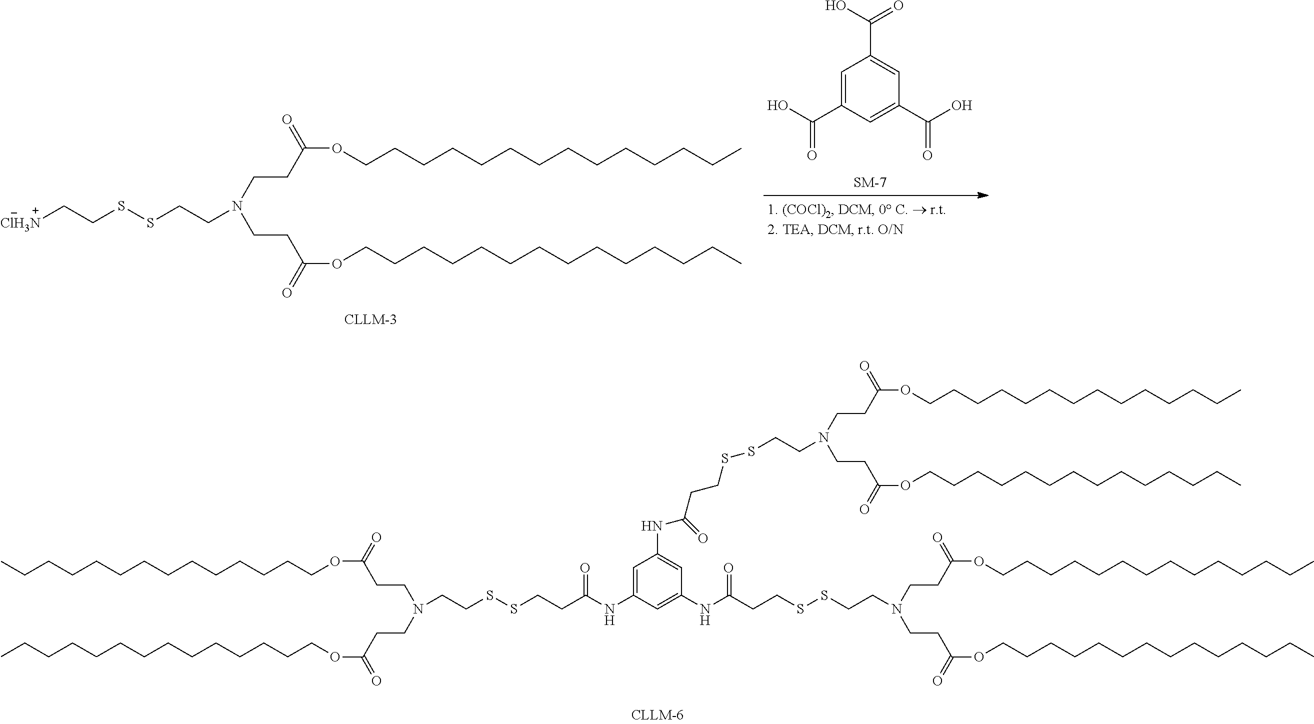

General procedure for the preparation of CLLM-6: A solution of SM-7 (200 mg, 0.95 mmol, 1.0 eq) in DCM (5 mL) at 0 oC were added DMF (3 drops) and oxalyl chloride (362 mg, 9.5 mmol, 10.0 eq). The reaction was stirred at 45 oC for 2 h. The mixture was concentrated under reduced. Toluene (10 mL) was added and vacuum-dried. After concentration, the yellow solid was used for next step without further purification.

To a solution of CLLM-3 (2.58g, 3.75 mmol, 5.0 eq) and triethylamine (682 mg 6.75 mmol, 9.0 eq) in DCM (10 mL) was added the acyl chloride prepared above. The reaction was stirred at rt overnight. The reaction was monitored by TLC, and quenched with water (30 mL) and extracted with DCM (30 mL×2). The organic phase was washed with brine, and dried over sodium sulfate and concentrated under reduced pressure. The residue was purified by FCC give CLLM-6 (139 mg, 9%) as white solid.

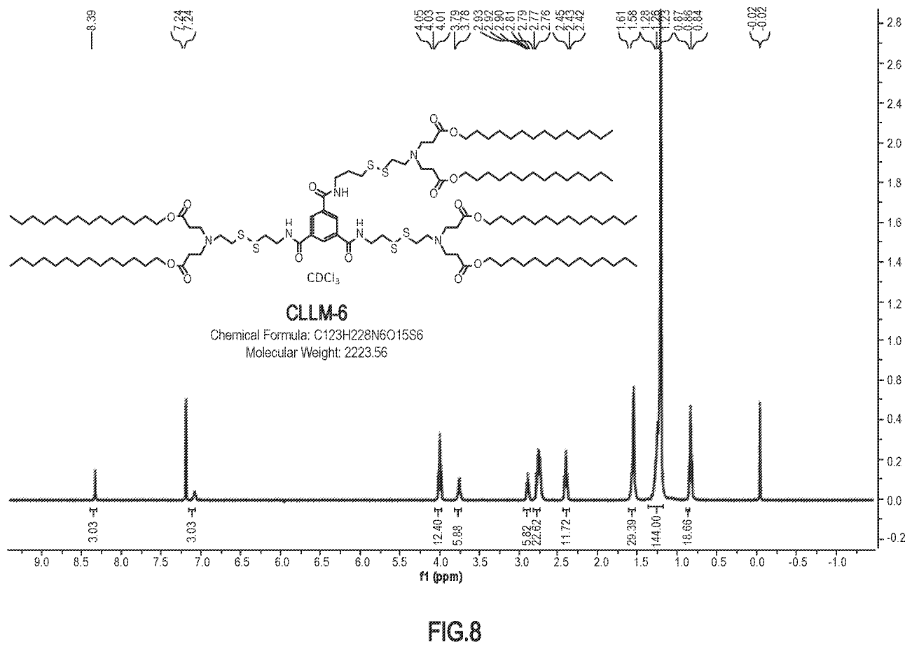

NMR characterization of CLLM-6 is summarized below and shown in FIG. 8.

¹ H NMR of CLLM-6 (400 MHz, CDCl₃): δ8.39 (s, 3H), 7.24 (s, 3H), 4.05-4.01 (t, 12H), 3.79-3.78 (m, 6H),2.93-2.90 (t, 6H), 2.81-2.76 (m, 22H), 2.45-2.42 (t, 12H), 1.61-1.58 (m, 29H),1.28-1.23(m, 144H), 0.87-0.84(t, 18H).

### II. Biological Evaluation

Three CDM compounds—CLLM-2, CLLM-4, and CLLM-6—were examined with respect to their abilities to package and deliver nucleic acid molecules into cells.

CLLM-2, CLLM-4, and CLLM-6 were synthesized through the processes in Examples 2, 4, and 6. 1,2-Dioleoyl-3-trimethylammonium propane (DOTAP) was purchased from Avanti Polar Lipids. Cholesterol was purchased from Sigma-Aldrich. 1,2-dioleoyl-sn-glycero-3-phosphoethanolamine (DOPE) and 1,2-Dimyristoyl-sn-glycerol-methoxypolyethylene glycol 2000 (DMG-PEG) were purchased from NOF America Corporation. Luciferase-encoded mRNA (mRNA-Luc) was gifted from Vernal Bioscience for testing its mRNA-Luc product within our CLLMs. Luciferase-encoded plasmid DNA (pDNA-Luc) was purchased from Aldevron. The 4T1 murine breast cancer cell line was purchased from ATCC.

### Example 7—Preparation of Nanoparticle Formulations of CDM Compounds

The two formulation compositions were prepared. Composition one (C #1) and Composition two (C #2) were formulated by dissolving CDM compounds, DOPE, cholesterol and DMG-PEG in ethanol. CLLM, DOPE, cholesterol, DMG-PEG and DOTAP were dissolved in ethanol at the molar ratio of 20/20/38/2/20. mRNA or pDNA dissolved in citrate buffer (10 mM, pH 4.0), weight ratio of 20:1 (total lipids:mRNA/pDNA), was pipette mixed rapidly into the lipids solution in ethanol at a volume ratio of 3:1 (mRNA/pDNA:lipids, v/v), then incubated for 10 min at room temperature. After formation, the fresh CLLM lipid nanoparticle (LNP) formulations were diluted with 1× PBS to 1 ng per 1 μl (with ethanol less than 5%) for in vitro assays and size measurement.

### Example 8—Characterization of Nanoparticle Formulations

To evaluate physicochemical properties of mRNA/pDNA-loaded CLLM nanoparticles formulations, the Tyndell effect of the particle solutions was examined, and the Dynamic Light Scattering (DLS, Malvern) was also used. Size in and Polydispersity Index (PDI) were measured using 200 μl fresh nanoparticles (1 ng mRNA/pDNA per μl, as described above), followed zeta-potential was tested by diluting with 1× PBS to 800 μl.

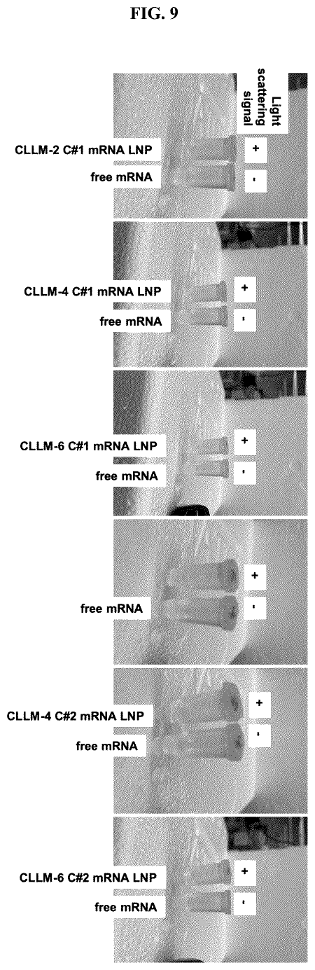

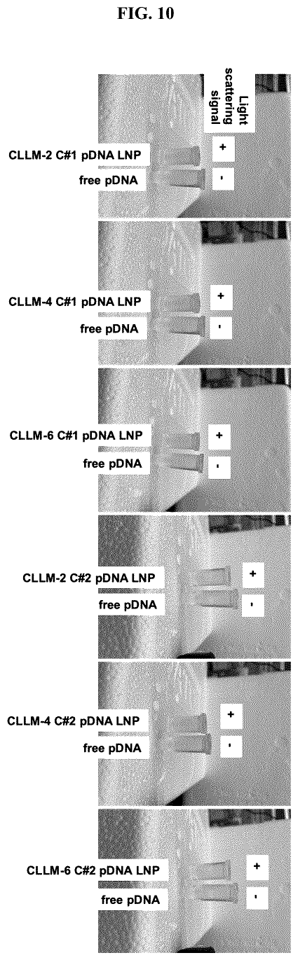

FIGS. 9 and 10 show that the Tyndell effect was not produced from the solutions of free mRNA or pDNA but from the LPN formulations, indicating that the particles are formed in the formulation process and resulted in the light scattering when accounting a light beam.

As shown in Table 3 below, CLLM-2, CLLM-4, and CLLM-6 formed relatively homogeneous lipid nanoparticles (LNPs) of diameter of 110-190 nm and zeta potential of negative 4-18 mV with both mRNA and pDNA (SD: standard deviation. PDI: polydispersity index).

TABLE 3
CDM Nanoparticle Characterization

| | Size in | | | | Zeta Potential | |
|---|---|---|---|---|---|---|
| | Diameter (nm) | SD (nm) | PDI | SD | (mV) | SD (mV) |
| CLLM-2 C#1 mRNA LNP | 136.9 | 0.6 | 0.13 | 0.01 | −8.7 | 6.4 |
| CLLM-4 C#1 mRNA LNP | 181.9 | 2.8 | 0.12 | 0.03 | −13.5 | 2.2 |
| CLLM-6 C#1 mRNA LNP | 151.9 | 3.5 | 0.14 | 0.01 | −17.7 | 2.3 |
| CLLM-2 C#2 mRNA LNP | 111.0 | 4.1 | 0.11 | 0.01 | −1.6 | 3.5 |
| CLLM-4 C#2 mRNA LNP | 157.4 | 3.7 | 0.10 | 0.00 | −4.7 | 3.4 |
| CLLM-6 C#2 mRNA LNP | 143.0 | 4.4 | 0.12 | 0.02 | −4.2 | 2.7 |
| CLLM-2 C#1 pDNA LNP | 128.9 | 6.0 | 0.09 | 0.01 | −6.1 | 4.3 |
| CLLM-4 C#1 pDNA LNP | 167.1 | 7.9 | 0.14 | 0.01 | −11.2 | 1.0 |
| CLLM-6 C#1 pDNA LNP | 158.7 | 3.7 | 0.16 | 0.02 | −11.0 | 1.4 |
| CLLM-2 C#2 pDNA LNP | 123.2 | 3.4 | 0.09 | 0.00 | −0.5 | 1.1 |
| CLLM-4 C#2 pDNA LNP | 156.4 | 5.4 | 0.08 | 0.01 | −11.1 | 7.5 |
| CLLM-6 C#2 pDNA LNP | 156.4 | 5.4 | 0.08 | 0.01 | −11.1 | 7.5 |

### Example 9—In Vitro Luciferase Expression and Cell Viability

4T1 murine breast cancer cells were seeded into white 96-well plate with the density of 1×104 cells per well. After 24 h, cells were replaced by 180 μL fresh RPMI 1640 medium (10% FBS), and 20 μL mRNA-Luc or Luc-pDNA encapsulated CLLM Nanoparticle formulations were added with fixed 100 ng mRNA or 200 ng pDNA per well. Free mRNA-Luc or Luc-pDNA were used as the control. Cells were further incubated for 24 h and ONE-Glo+Tox kits were used for mRNA expression and cytotoxicity detection based on the standard protocol.

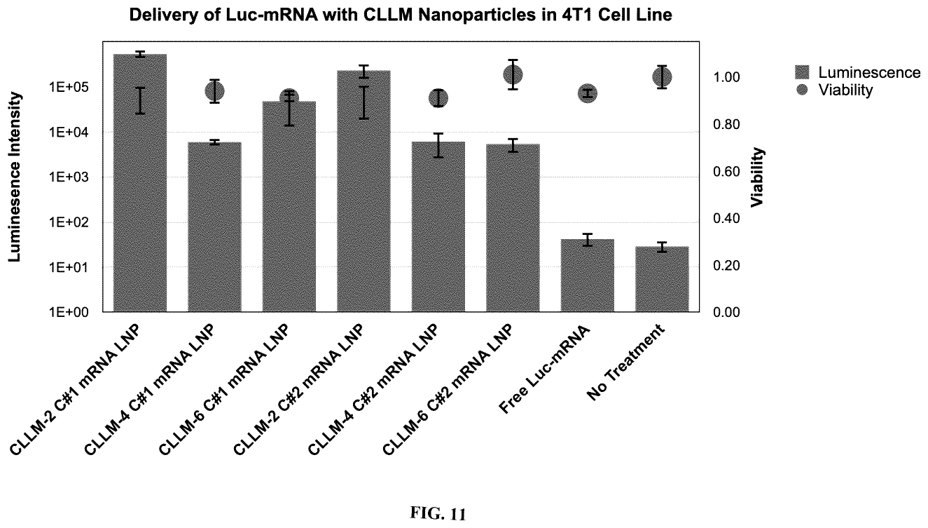

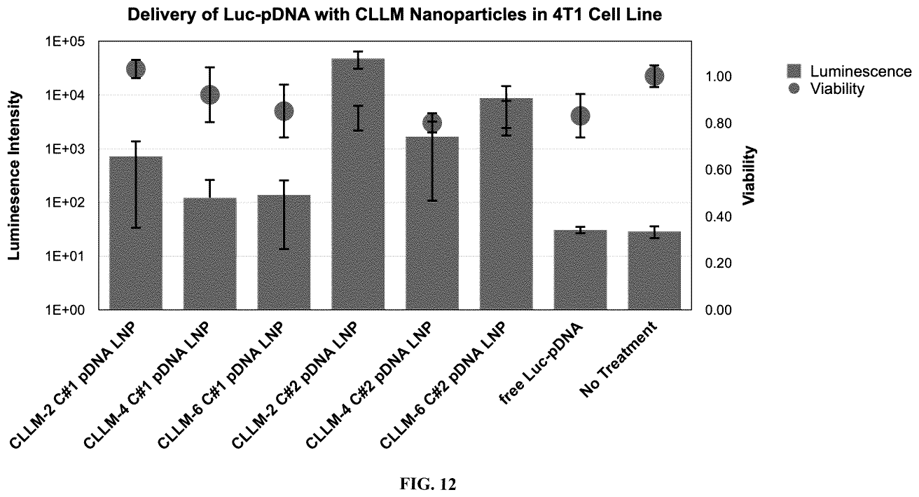

To examine whether CLLM-2, CLLM-4, and CLLM-6 can deliver mRNA and pDNA into cells, the CLLM LNPs formulated with either Luciferase-encoded mRNA or pDNA at two compositions were incubated with 4T1 murine breast cancer cells for 24 hours. FIGS. 11 and 12 show the 4T1 cells incubated with CLLM Luciferase-encoded mRNA or pDNA LNPs have luciferase expression, indicating that CLLM-2, CLLM-4, and CLLM-6 can deliver mRNA and pDNA into cells. The 4T1 cells incubated with free mRNA or pDNA do not have luciferase expression, confirming that mRNA or pDNA needs to helped to enter the cells. The CLLM LNPs were very tolerated in 4T1 cells. Interestingly, the CLLM LNPs of C#2 composition that contains DOTAP having permanently positive charge do not enhance the delivery of mRNA into cells but do enhance the delivery of pDNA. It indicates that requirement of delivering pDNA is different from that of delivering mRNA although they are nucleic acid molecules.

In this non-limiting example, all three CDM compounds formed nanoparticles with both luciferase-encoded mRNA and pDNA. The nanoparticle formulations delivered both luciferase-encoded mRNA and pDNA into 4T1 murine breast cancer cells.

What is claimed is:

1. A compound of Formula I, a stereoisomer or pharmaceutically acceptable salt thereof:
RₐR_{b}N-L₁-S—S-L₂-NR_{c}R_{d} (Formula I)
wherein L₁ and L₂ are each independently C₁₋₄ alkylenyl;
wherein at least one of Rₐ, R_{b}, R_{c} and R_{d} is independently a hydrophobic tail moiety (HTM) or combined with nitrogen on the same side of Formula I to form a 3-member to 8-member heterocyclic ring substituted with an HTM, and the wherein the remainder of Rₐ, R_{b}, R_{c} and R_{d} is independently a hydrogen, a C₁₋₄ alkyl, or combine with nitrogen on the same side of Formula I to form a 3-member to 8-member heterocyclic ring optionally substituted with a C₁₋₄ alkyl,
wherein HTM is a C₅₋₄₀ linear or branched alkyl, C₅₋₄₀ linear or branched alkenyl, or C₅₋₄₀ linear or branched alkynyl; or wherein HTM has a structure of Formula II:
-L-NR′R″ (Formula II)
wherein L is C₁₋₄ alkylenyl and at least one of R′ and R″ is a C₅₋₄₅ linear or branched alkyl, C₅₋₄₅ linear or branched alkenyl, or C₅₋₄₅ linear or branched alkynyl.

2. The compound of claim 1, wherein L₁ and L₂ are each independently C₁₋₃ alkylenyl.

3. The compound of claim 2, wherein L1 is ethylene.

4. The compound of claim 3, wherein L2 is ethylene.

5. The compound of claim 4, wherein Rₐ and R_{b} is independently a hydrogen, a C₁₋₄ alkyl, or combine with nitrogen on the same side of Formula I to form a 3-member to 8-member heterocyclic ring optionally substituted with a C₁₋₄ alkyl.

6. The compound of claim 5, wherein Rₐ and R_{b} are hydrogen.

7. The compound of claim 5, wherein the Rₐ is hydrogen and wherein R_{b} is a C₁₋₄ alkyl.

8. The compound of claim 7, wherein R_{b} is a butyl.

9. The compound of claim 5, wherein Rₐ and R_{b} are each independently a C₁₋₄ alkyl.

10. The compound of claim 5, wherein Rₐ and R_{b} are methyl.

11. The compound of claim 5, wherein Rₐ and R_{b} combine with nitrogen on the same side of Formula I to form a 3-member to 8-member heterocyclic ring.

12. The compound of claim 11, wherein the heterocyclic ring is substituted with a C₁₋₄ alkyl.

13. The compound of claim 11, wherein the heterocyclic ring is a diazinane ring.

14. The compound of claim 13, wherein the diazinane ring is a 1,4-diazinane ring.

15. The compound of claim 14, wherein the 1,4-diazinane ring is substituted with a C₁₋₄ alkyl at the 4 position.

16. The compound of claim 5, wherein R_{c} is an HTM and R_{d} is a hydrogen or a C₁₋₄ alkyl.

17. The compound of claim 5, wherein R_{c} and R_{d} are each independently an HTM.

18. The compound of claim 5, wherein R_{c} and R_{d} combined with nitrogen on the same side of Formula I to form a 3-member to 8-member heterocyclic ring substituted with an HTM.

19. The compound of claim 18, wherein the heterocyclic ring is a diazinane ring.

20. The compound of claim 19, wherein the diazinane ring is a 1,4-diazinane ring.

21. The compound of claim 20, wherein the 1,4-diazinane ring is substituted with the HTM at the 4 position.

22. The compound of claim 1, wherein at least one of Rₐ and R_{b} is an HTM or Rₐ and R_{b} combine with nitrogen on the same side of Formula one to form a 3-member to 8-member heterocyclic ring substituted with an HTM, and wherein at least one of R_{c} and R_{d} is an HTM or R_{c} and R_{d} combine with nitrogen on the same side of Formula one to form a 3-member to 8-member heterocyclic ring substituted with an HTM.

23. The compound of claim 1, wherein HTM is a C₅₋₄₀ linear or branched alkyl.

24. The compound of claim 23, wherein HTM is a C₁₀₋₂₀ linear alkyl.

25. The compound of claim 23, wherein HTM is a C₁₀₋₃₀ branched alkyl and optionally wherein the branched alkyl has one C₅₋₁₅ alkyl branch on a C₅₋₁₅ alkyl main chain.

26. The compound of claim 23, wherein HTM is a C₁₅₋₄₀ branched alkyl and optionally wherein the branched alkyl has two C₅₋₁₅ alkyl branches on a C₅₋₁₅ alkyl main chain.

27. The compound of claim 23, wherein the linear or branched alkyl has one or more intervening groups selected from ester —(C═O)—O—, amide —(C═O)—NR—, or carboxylate —O—(C═O)—O—, wherein R is hydrogen, a C₁₋₄ alkyl, C₁₋₄ alkenyl, or C₁₋₄ alkynyl.

28. The compound of claim 27, wherein the one or more intervening group is inserted in the alkyl such that the acyl group is connect to nitrogen in Formula I or Formula II through an ethylene —CH₂—CH₂—.

29. The compounds of claim 1, wherein HTM has the structure of 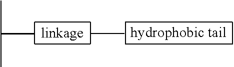
wherein the linkage is selected from 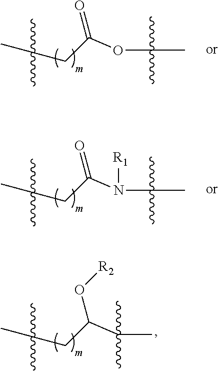
m is an integer of 1-6; R₁ is H, CH₃, or CH₂CH₃; and R₂ is H, CH₃, CH₂CH₃, or (C═O)CH₃, wherein the hydrophobic tail is selected from the group consisting of:

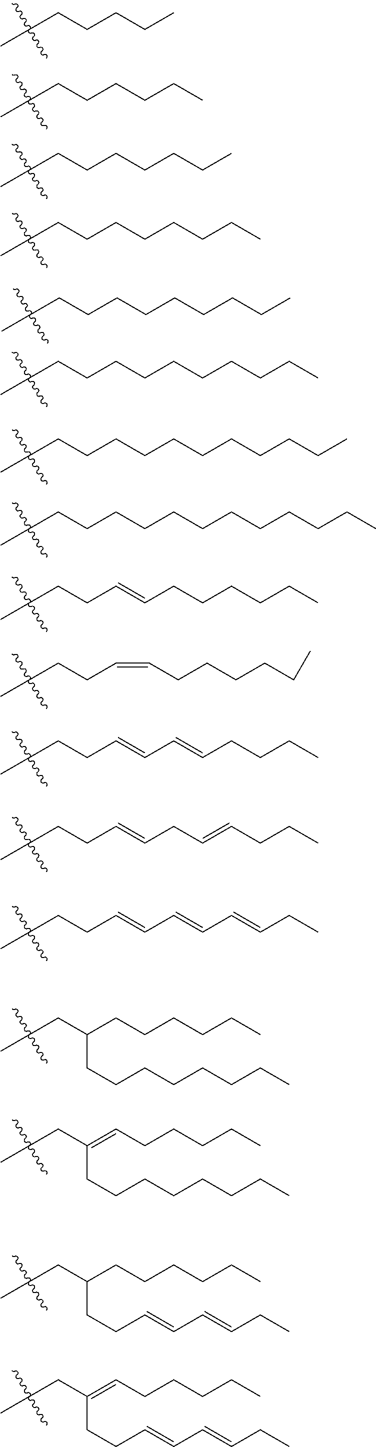

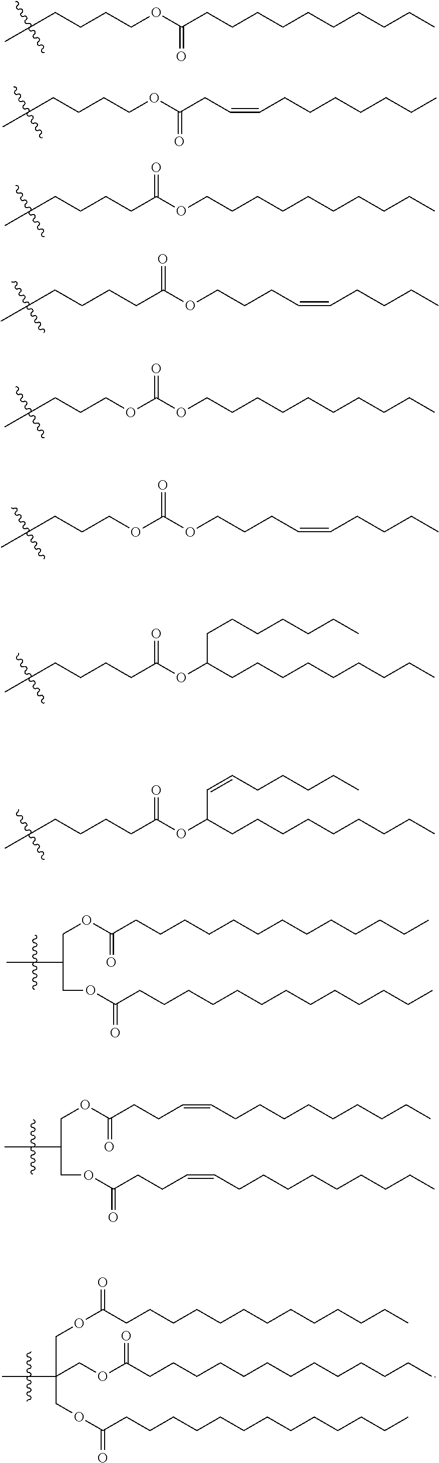

30. The compound of claim 1, selected from the group consisting of: 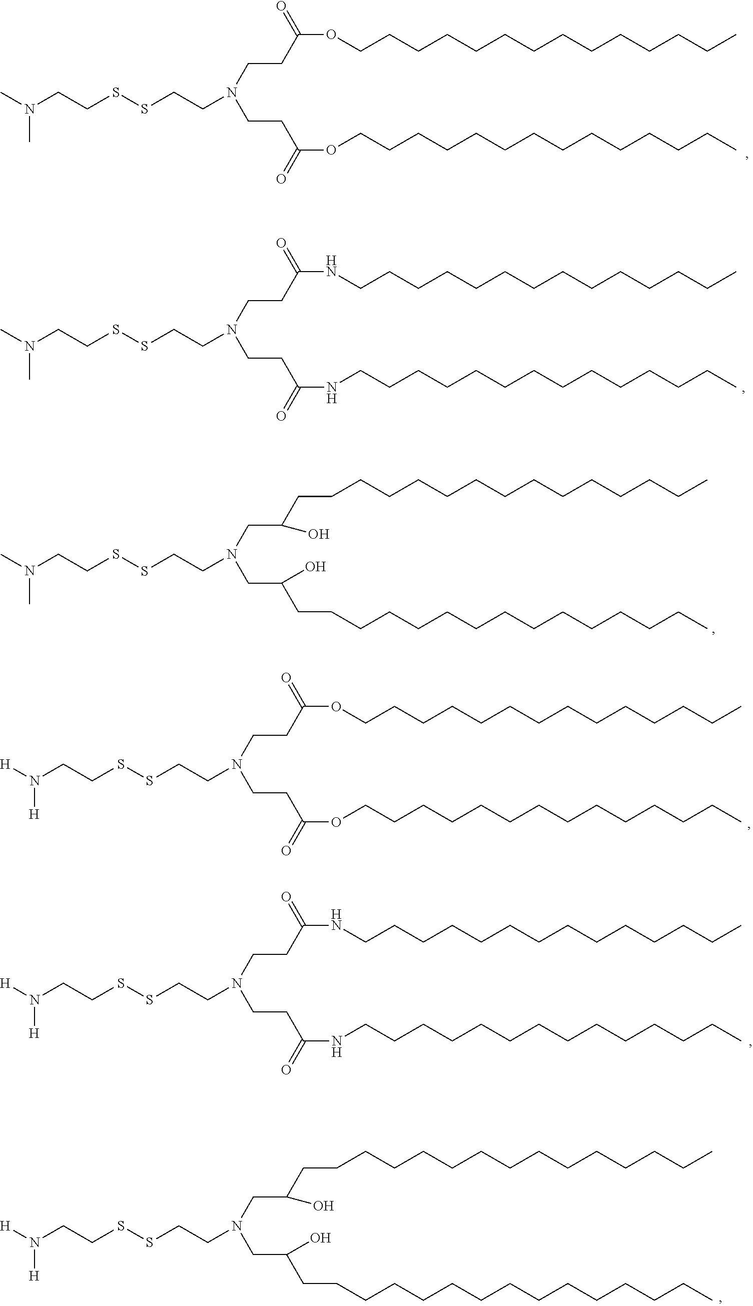 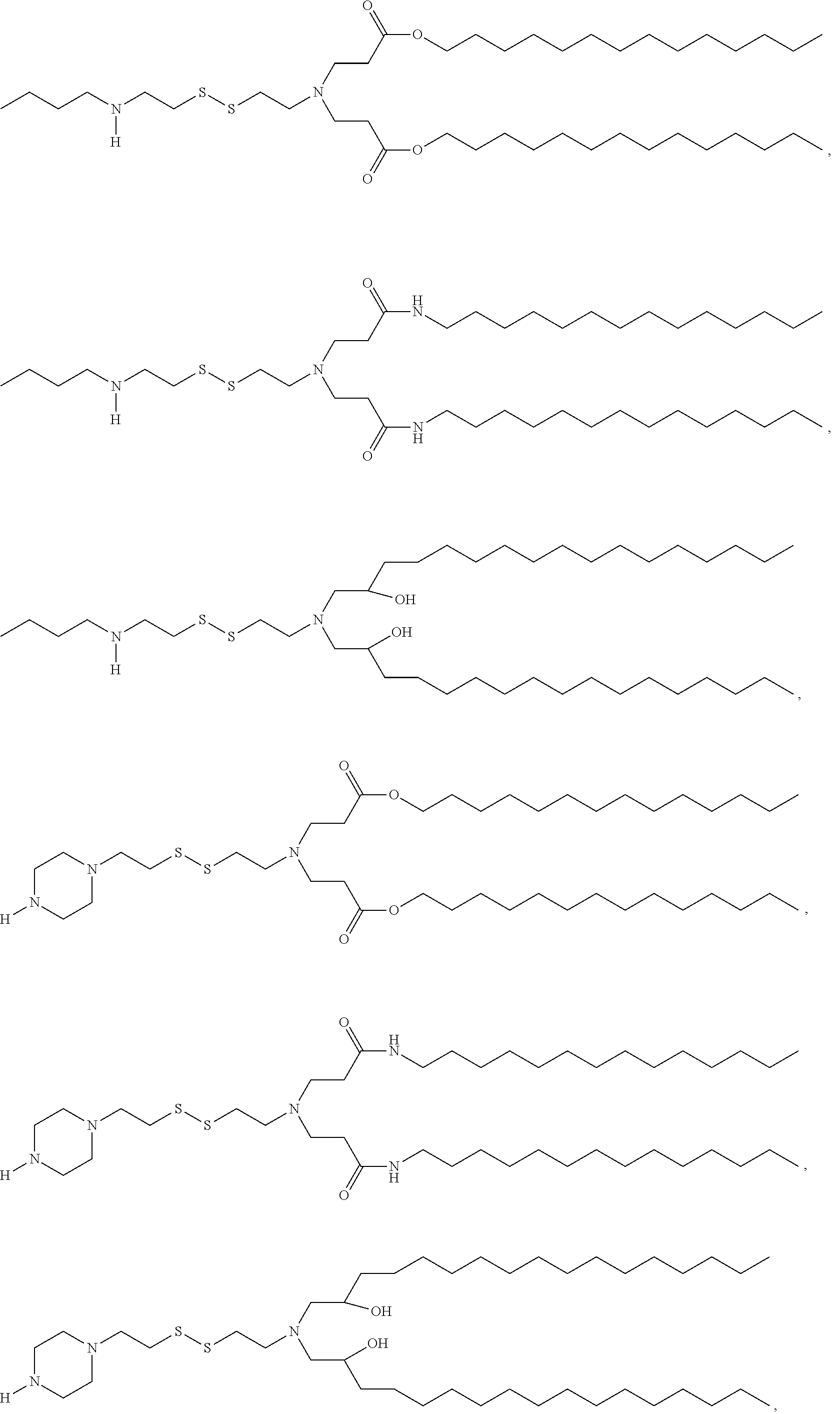 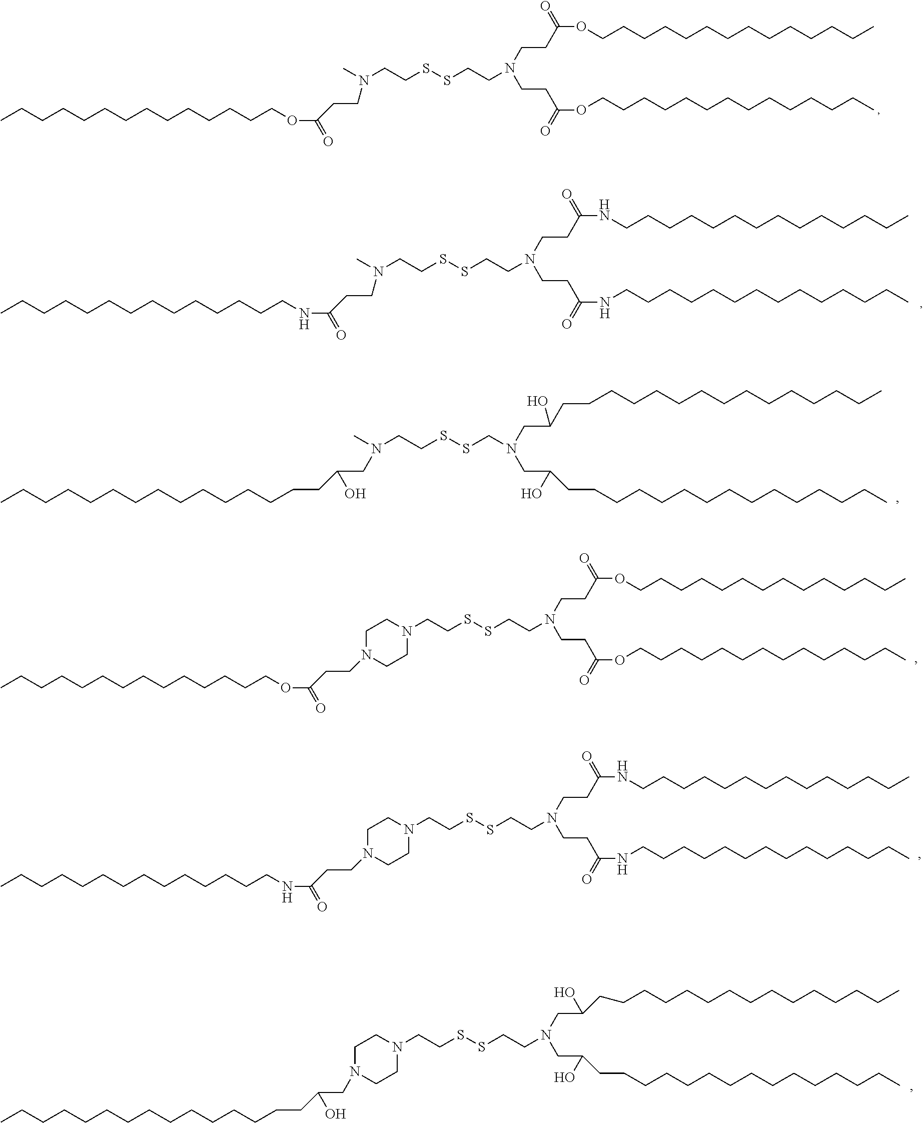 [chemical structure]
